(19) United States
(12) Patent Application Publication
(10) Pub. No.: US 2022/0073985 A1
(43) Pub. Date: Mar. 10, 2022

(54) DISEASE STRATIFICATION OF LIVER DISEASE AND RELATED METHODS

(71) Applicant: Molecular Stethoscope, Inc., San Diego, CA (US)

(72) Inventors: Michael Nerenberg, San Diego, CA (US); Arkaitz Ibarra, San Diego, CA (US); Jiali Zhuang, San Diego, CA (US); Shusuke Toden, San Diego, CA (US); Guillermo Elias, San Diego, CA (US)

(21) Appl. No.: 17/239,461

(22) Filed: Apr. 23, 2021

Related U.S. Application Data
(63) Continuation of application No. PCT/US2019/058209, filed on Oct. 25, 2019.
(60) Provisional application No. 62/818,612, filed on Mar. 14, 2019.
(60) Provisional application No. 62/751,407, filed on Oct. 26, 2018.

(51) Int. Cl. C12Q 1/6883

(52) U.S. Cl. CPC C12Q 1/6883; C12Q 2600/158; C12Q 2600/112

(57) ABSTRACT

Methods of assessing or determining a disease stage of a liver are provided. The methods can include obtaining a sample from a subject. The methods can also include measuring gene expression products in the sample from the subject to determine the disease stage of the liver.

### CROSS-REFERENCE

This application claims the benefit of U.S. Provisional Application No. 62/751,407, filed Oct. 26, 2018, and U.S. Provisional Application No. 62/818,612, filed Mar. 14, 2019, each of which is entirely incorporated herein by reference.

### BACKGROUND

Histological examination of liver biopsy tissue can be used for diagnosing stages of NAFLD, and guidelines for best practices in liver disease diagnosis have been established. Meta-analyses have revealed that amongst clinical parameters known to be associated with NAFLD diagnosis, such as NASH status, NAS (NAFLD Activity Score) and liver histological features, fibrosis staging can be the primary predictor of mortality and time to liver decompensation in NAFLD patient. Therefore, the ability to identify fibrosis stages can be used in managing patient health. Liver fibrosis can be divided into four stages: no observable fibrosis (F0), portal fibrosis without septa (F1), portal fibrosis with few septa (F2), bridging septa between central and portal veins (F3), and cirrhosis (F4). Several non-invasive ways of assessing NAFLD and associated fibrosis have been reported. The FIB-4 (Fibrosis-4) index, derived from measurements of patient age, aspartate, and alanine aminotransferase levels and the platelet count, can be used to predict fibrosis. More recently, VCTE (Vibration-Controlled Transient Elastography) ultrasound analysis has been FDA approved for accurately assessing liver fibrosis state.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

Disclosed herein is a method of assessing a disease state of a liver. The method can comprise obtaining or having obtained a sample from a subject, wherein the sample may comprise gene expression products, measuring gene expression products of a panel of genes comprising at least one gene selected from Table 3, Table 4, Table 5, Table 6, Table 7 and/or Table 10 and determining a disease state of the liver. The at least one gene can be at least one of PITPNM2, LIMCHI1, FSCN1, CCND1, or CASKIN2. The sample can be selected from saliva, blood, sputum, urine, semen, transvaginal fluid, sweat, breast milk, breast fluid, stool, a cell, or a tissue biopsy. The sample can be blood plasma. The panel of genes can comprise at least 5 genes. The panel of genes can comprise at least 50 genes. The panel of genes can comprise at least 100 genes. The panel of genes can comprise at least 200 genes. The gene expression products can be protein. The gene expression products can be RNA. The RNA can be cell-free messenger RNA. Measuring gene expression products can comprise one or more of sequencing, array hybridization, or nucleic acid amplification. Measuring gene expression products can comprise reverse transcription of the cell-free messenger RNA to cDNA, amplifying the cDNA to produce amplified cDNA, and using the amplified cDNA to probe a microarray containing gene transcripts associated with the disease state of the liver. Determining can comprise a trained classifier to generate a classification of the sample as indicating the disease state of the liver. Determining can comprise a trained classifier to generate a classification of the sample as indicating the disease state of the liver. The trained classifier can be trained by a training set comprising blood samples from subjects with biopsy verified diagnosis of liver disease. The disease state can be selected from the group consisting of no observable fibrosis (F0), portal fibrosis without septa (F1), portal fibrosis with few septa (F2), bridging septa between central and portal veins (F3), and cirrhosis (F4). The method can further comprise diagnosing a disease of the liver. The method can further comprise prescribing a course of treatment. The course of treatment can comprise dietary changes, administration of a pharmaceutical, or administration of a dietary supplement. The disease can be nonalcoholic fatty liver disease (NAFLD). The disease can be nonalcoholic steatosis (NAFL). The disease can be nonalcoholic steatohepatitis (NASH).

Disclosed herein is a method for processing or analyzing a sample from subject, the method comprising: (a) obtaining or having obtained a sample from a subject, wherein the sample comprises gene expression products; (b) assaying the gene expression products to yield data corresponding to an expression level of one or more gene expression products in the data, wherein the one or more gene expression products are associated with a liver disease state; (c) in a programmed computer, inputting the data including the expression level of one or more gene expression products from (b) to a trained classifier to generate a classification of the sample as indicating a liver disease state; and (d) electronically outputting a report that identifies the classification of the sample as indicating a liver disease state. The assaying of (b) can comprise at least one of sequencing, array hybridization, or nucleic acid amplification. The gene expression products can be cell-free messenger RNA. The sample can be selected from saliva, blood, sputum, urine, semen, transvaginal fluid, sweat, breast milk, breast fluid, stool, a cell, or a tissue biopsy. The sample can be blood plasma. The trained classifier can be trained by a training set comprising blood samples from subjects with a biopsy verified diagnosis of liver disease. The gene expression products can be RNA. The gene expression products can be mRNA. The gene expression products can be cell-free mRNA. The sequencing can comprise reverse transcription of the cell-free messenger RNA to cDNA, amplifying the cDNA to produce amplified cDNA, and using the amplified cDNA to probe a microarray containing gene transcripts associated with the liver disease state. The one or more gene expression products can be highly expressed in endothelial cells. The one or more gene expression products can be related to at least one of blood vessel development, vasculature, and angiogenic processes. The sequencing can comprise whole-transcriptome analysis further comprising a next-generation sequencing platform. The assaying of (b) can further comprise assaying gene expression products comprising liver-specific transcripts. The assaying of (b) can further comprise assaying gene expression products comprising one or more genes from Table 7 and/or Table 10. The one or more genes can be selected from the group consisting of PITPNM2, LIMCH1, CCND1, and CASKIN2. The specific cell types can comprise red blood cells (RBC), polymorphonuclear leukocytes (PMN), platelets, and liver cells, hepatic stellate cells, hepatocytes. The trained classifier can comprise a logistic regression model. The liver disease state can be selected from the group consisting of no observable fibrosis (F0), portal fibrosis without septa (F1), portal fibrosis with few septa (F2), bridging septa between central and portal veins (F3), and cirrhosis (F4).

Disclosed herein is a method for treating a patient with a liver disease, the method comprising (a) obtaining or having obtained a biological sample comprising gene expression products from the patient; (b) performing or having performed analysis of the gene expression products to determine if the patient has the liver disease; and (c) recommending a treatment for the liver disease. The analysis can comprise at least one of sequencing, array hybridization, or nucleic acid amplification. The gene expression products can be RNA. The gene expression products can be cell-free mRNA. The liver disease can be selected from the group consisting of no observable fibrosis (F0), portal fibrosis without septa (F1), portal fibrosis with few septa (F2), bridging septa between central and portal veins (F3), and cirrhosis (F4). The sample can be selected from saliva, blood, sputum, urine, semen, transvaginal fluid, sweat, breast milk, breast fluid, stool, a cell, or a tissue biopsy. The treatment can comprise dietary changes, administration of a pharmaceutical, or administration of a dietary supplement. The method can further comprise in (b) determining a stage of the liver disease. The method can further comprise (d) administering the treatment and (e) monitoring a liver disease progression of the patient wherein the biological sample is a first sample and the stage of the liver disease is a first stage of the liver disease, and wherein monitoring comprises (i) obtaining or having obtained a second sample comprising gene expression products from the patient; (ii) performing or having performed analysis of the gene expression products to determine a second stage of the liver disease; and (iii) comparing the first stage of the liver disease to the second stage of the liver disease.

Disclosed herein is a system to identify a liver disease from a biological sample, the system comprising (a) a classifier comprising a gene expression panel further comprising at least one gene selected from Table 3, Table 4, Table 5, Table 6, Table 7 and/or Table 10; and (b) a computer system configured to apply the classifier to a gene expression profile of a biological sample. The at least one gene can comprise at least one of PITPNM2, LIMCH1, FSCN1, CCND1, and CASKIN2. The classifier can comprise at least two genes, at least three genes, at least four genes, or at least five genes from Table 3, Table 4, Table 5, Table 6, Table 7 and/or Table 10. The gene expression profile can comprise at least one of sequencing data, array hybridization data, or nucleic acid amplification product data. The gene expression profile can comprise sequencing data and further comprise levels of gene transcripts. The gene expression profile can comprise values corresponding to levels of gene transcripts of cell-free mRNA. The classifier can scale the values corresponding to levels of gene transcripts of cell-free mRNA to housekeeping gene transcript levels. The biological sample can be selected from saliva, blood, sputum, urine, semen, transvaginal fluid, sweat, breast milk, breast fluid, stool, a cell, or a tissue biopsy. The liver disease can be selected from the group consisting of no observable fibrosis (F0), portal fibrosis without septa (F1), portal fibrosis with few septa (F2), bridging septa between central and portal veins (F3), and cirrhosis (F4). The classifier can comprise a non-negative matrix factorization (NMF) to classify gene expression products of the gene expression profile as associated with specific cell types.

Disclosed herein is a method for detecting a disease state of a liver, the method comprising: (a) determining an expression level of one or more markers in a sample obtained from a subject, wherein the one or more markers are selected from Table 3, Table 4, Table 5, Table 6, Table 7 and/or Table 10; and (b) comparing the expression level to a reference level of the one or more markers; wherein an increased or decreased expression level of the one or more markers relative to the reference expression level indicates that the subject has the disease state. The reference level can be obtained from a healthy control subject or an average level from a group of healthy control subjects. The disease state can be at least one of no observable fibrosis (F0), portal fibrosis without septa (F1), portal fibrosis with few septa (F2), bridging septa between central and portal veins (F3), or cirrhosis (F4). The method can further comprise obtaining two or more samples from the same subject, wherein the two or more samples are from different time points, and wherein (a) is repeated for each of the two or more samples and wherein the expression level for each of the two or more samples are compared in step (b). The method can further comprise treating the subject for the disease state if the increased or decreased expression level of the one or more markers relative to the reference expression level indicates that said subject has the disease state. The treatment can be selected from the group consisting of administering a therapeutic agent, administering a surgical intervention, or a combination thereof. The therapeutic agent can be selected from the group consisting of drugs targeting metabolism of lipids, metabolism of glucose, drugs targeting metabolic inflexibility, drugs targeting fibrosis, anti-inflammatory compounds, acetyl-CoA Carboxylase inhibitor, OCA, elafibranor cenicrivaroc, vitamin-e, plioglitazoe, PPAR agonist, FXR agonist, ASK-1 inhibitor, fibroblasts growth factors, insulin sensitizer or bile acid regulator. The surgical intervention can be selected from the group consisting of weight loss associated surgery, liver resection and liver transplantation. The method can further comprise placing the subject in a non-treatment category if the subject does not have increased or decreased expression level of the one or more markers relative to the reference expression level.

Disclosed herein is a method for detecting a disease state of a liver, the method comprising (a) determining an expression level of one or more markers in a sample obtained from a subject, wherein the one or more markers are selected from Table 3, Table 4, Table 5, Table 6, Table 7 and/or Table 10; (b) applying a classifier algorithm to the expression level of one or more markers and a reference level of each of the one or more markers to calculate a metric that quantifies a difference between the expression level and the reference level for each of the one or more markers; and (c) determining a disease state of a liver based on the metric.

Disclosed herein is a method of assaying an active agent comprising (a) assessing a first cell-free expression profile of a subject at a first time point; (b) administering an active agent to the subject; and (c) assessing a second cell-free expression profile of the subject at a second time point. The method can further comprise comparing the first cell-free expression profile to the second cell-free expression profile. The difference between the first expression profile and the second expression profile can indicate an effect of the therapy. The active agent can be a pharmaceutical compound to treat a disease. The method can further comprise assessing a third cell-free expression profile of a subject at a third time point. Assessing can comprise one or more of sequencing, array hybridization, or nucleic acid amplification. The method can further comprise assessing additional cell-free expression profiles of the subject at additional time points. The second time point can be from one to four weeks after the first time point. The method can further comprise assessing the additional cell-free expression time points over a period from 12 to 24 months. The period can be about 18 months. The method can further comprise tracking and/or detecting one or more cell-free expression profiles to measure one or more targets of interest for therapy and/or drug discovery and/or development. The method can further comprise measuring pharmacodynamics for a lead optimization and/or a clinical development during therapy and/or drug discovery and development. The method can further comprise creating a profile of gene expression to characterize one or more pharmacodynamic effects associated with an engagement of a specific target for therapy and/or drug discovery and/or development. The method can further comprise detecting changes in pharmacodynamics target engagement for therapy and/or drug discovery and development.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A shows comparison between sequencing transcripts per million (TPM) against qPCR Ct value (96 genes in 61 individuals).

FIG. 1B shows a schematic for comparison between next generation sequencing assays and qPCR performed in FIG. 1A.

FIG. 2A shows exemplary key patient clinical liver disease state characteristics in the 3 patient cohorts tested.

FIG. 2B shows exemplary key patient clinical fibrosis stage breakdown characteristics in the 3 patient cohorts tested.

FIG. 2C shows exemplary key patient clinical steatosis characteristics in the 3 patient cohorts tested.

FIG. 2D shows exemplary key patient clinical ballooning characteristics in the 3 patient cohorts tested.

FIG. 3A shows additional exemplary key diabetes patient clinical characteristics in the 3 patient cohorts tested.

FIG. 3B shows additional exemplary key BMI patient clinical characteristics in the 3 patient cohorts tested.

FIG. 3C shows additional exemplary key inflammation patient clinical characteristics in the 3 patient cohorts tested.

FIG. 3D shows another additional key platelet count patient clinical characteristics in the 3 patient cohorts tested.

FIG. 3E shows another additional key AST patient clinical characteristics in the 3 patient cohorts tested.

FIG. 3F shows another additional key ALT patient clinical characteristics in the 3 patient cohorts tested.

FIG. 4A shows a histogram representing correlation between the cf-mRNA transcriptomes of technical replicates using Pearson's correlation analysis.

FIG. 4B show a histogram of detection sensitivity as measured by copy number detection threshold of ERCC transcripts.

FIG. 4C shows a histogram of Pearson's correlation coefficient between observed and expected expression levels of ERCC reference genes.

FIG. 4D shows a histogram of the number of transcripts detected (TPM>5) per sample.

FIG. 5 shows intra- vs. inter-sample cf-mRNA profile variability with Principal Component Analysis (PCA) analysis; in this case, first two components (PC1 and PC2) of gene-expression PCA for 20 randomly chosen samples from the training cohort (5 individuals representing: non-liver disease, NAFL, low fibrosis NASH and high fibrosis NASH liver disease categories), wherein grey dots represent technical replicate 1 and black dots represent replicate 2 of the same serum sample and replicates of the same sample are close to each other.

FIG. 6A shows a volcano plot depicting the differential expression analysis in cf-mRNA between NASH and healthy controls. Significantly dysregulated genes are shown in the dotted boxes, FDR<0.05 and fold change >1.4 was used as the cut-off criteria.

FIG. 6B shows a graph of the top 5 upregulated and downregulated canonical pathways identified using genes significantly dysregulated in NASH. The black vertical dotted line represents significance threshold (adjusted p<0.05).

FIG. 6C shows a scatter plot demonstrating a systematic up-regulation of liver specific transcript levels in NASH patients compared to normal controls.

FIG. 6D shows a consensus matrix non-negative matrix factorization (NMF) clustering of all samples, functional analyses of gene clusters was performed and five major clusters are labeled accordingly.

FIG. 6E shows a volcano plot depicting the differentially expressed genes in in NAFLD patients with advanced fibrosis compared to patients with early fibrosis. The black horizontal dotted line represents a significance threshold (adjusted p<0.05).

FIG. 6F shows the top pathways enriched in genes upregulated in cf-RNA of patients with fibrosis stage F3/F4. The black vertical dotted line represents significance threshold (adjusted p<0.05).

FIG. 7 shows exemplary enrichment of liver-specific genes in component 6 identified in FIG. 6D RNA the row labeled liver is marked by a box around ‘liver.’FIG. 8A is a schematic of a study design as disclosed in embodiments herein.

FIG. 8B shows a ROC curve of cf-mRNA based classifier discriminating NAFL to healthy control using serum samples from the training cohort, wherein shaded regions represent AUC standard error generated from iterative cross-validation.

FIG. 8C shows a ROC curve of a cf-mRNA classifier discriminating NASH from healthy control using samples from the Training cohort, wherein shaded regions represent AUC standard error generated from iterative cross-validation.

FIG. 8D shows a ROC curve of a cf-mRNA classifier discriminating NASH to NAFL, using samples part of the Training cohort, wherein shaded regions represent AUC standard error generated from iterative cross-validation.

FIG. 9A shows ROC curve of a cf-mRNA classifier discriminating “early” (F0, F1) vs. “advanced” fibrosis (F3, F4) using samples in the training cohort. Error bars in Training cohort dataset represent AUC standard error generated from iterative cross-validation.

FIG. 9B shows another exemplary fibrosis classifier disclosed herein; in this case, performance of NAFL liver disease classifier to stratify fibrosis staging, “early” (F0, F1) vs. “advanced” fibrosis (F3, F4) in 3 patient cohorts. Error bars in Training cohort dataset represent AUC standard error generated from iterative cross-validation.

FIG. 9C shows yet another exemplary fibrosis classifier disclosed herein; in this case, performance of NAFL liver disease classifier to stratify fibrosis staging, “early” (F0, F1) vs. “advanced” fibrosis (F3, F4) in 3 patient cohorts. Error bars in Training cohort dataset represent AUC standard error generated from iterative cross-validation.

FIG. 10A shown an exemplary 5-gene classifier AUC; in this case, fibrosis stage classification (F0, F1 vs. F3, F4) using a 5-gene model using a Logistic Regression model.

FIG. 10B shows exemplary cf-mRNA gene-expression by fibrosis stage.

FIG. 10C shows yet another exemplary cf-mRNA gene-expression of genes by fibrosis stage.

FIG. 10D shows yet another exemplary cf-mRNA gene-expression of genes by fibrosis stage.

FIG. 10E shows yet another exemplary cf-mRNA gene-expression of genes by fibrosis stage.

FIG. 10F shows yet another exemplary cf-mRNA gene-expression of genes by fibrosis stage.

FIG. 11 shows top informative fibrosis classifier genes upregulated in advanced fibrosis are enriched in NMF-derived component 10; in this case, loading fractions of the 50 most informative genes in the fibrosis classifier that are upregulated in advanced fibrosis, distributed across all 12 NMF-derived components.

FIG. 12 shows component 10 genes that are enriched in endothelial cell transcript (Blueprint database).

FIG. 13A shows enrichment of endothelial genes in cf-mRNA fraction vs. peripheral blood compartment for a first individual; in this case, gene-expression of genes from NMF-derived component 10 in plasma vs. peripheral blood fractions, from non-liver diseased individuals. Genes with component loading fractions >0.45 and TPM >8 shown.

FIG. 13B shows enrichment of to the cf-mRNA fraction vs. peripheral blood compartment for a second individual; in this case, gene-expression of genes from NMF-derived component 10 in plasma vs. peripheral blood fractions, from 3 non-liver diseased individuals. Genes with component loading fractions >0.45 and TPM >8 shown.

FIG. 13C shows enrichment of genes to the cf-mRNA fraction vs. peripheral blood compartment for a third individual; in this case, gene-expression of genes from NMF-derived component 10 in plasma vs. peripheral blood fractions, from 3 non-liver diseased individuals. Genes with component loading fractions >0.45 and TPM >8 shown.

FIG. 14A shows a schematic of an exemplary study design.

FIG. 14B validation of the cf-mRNA based classifier for fibrosis stratification in NAFL/NASH patients from FIG. 9A, shows a ROC curve of cf-mRNA classifier

FIG. 14C shows a tabular summary of a fibrosis classifier cohort breakdown and performance.

FIG. 15A shows correlation between expected copy numbers of spiked in ERCC and their observed expression levels (TPM).

FIG. 15B shows graphs of average read coverage across exon-intron junctions.

FIG. 16A shows a volcano plot depicting the differential expression analysis in cf-mRNA between NAFL and healthy controls. Significantly dysregulated genes are denoted in grey squares, FDR<0.05 and fold change >1.41 was used as the cut-off criteria.

FIG. 16B shows a graph of the most significantly enriched pathways identified using genes significantly dysregulated in NAFL. The black vertical dotted line represents significance threshold (adjusted p<0.05).

FIG. 16C shows graphs of the expression levels of three liver specific transcripts in serums from normal controls and NASH patients.

FIG. 16D shows a graph of the number of liver specific genes detected in subjects with different liver disease status.

FIG. 16E shows a graph of expression levels of FSCN1 according to fibrosis stages.

FIG. 16F shows a graph of the coefficients of genes within the inflammation component correlated with a liver lobular inflammation score.

FIG. 17 shows performance of the classifier to discriminate NASH from NAFL, specifically among patients with mild fibrosis (F0-F1) average ROC curve of classifications distinguishing NASH from NAFL patients with low fibrosis is shown.

FIG. 18 shows a ROC curve of a cf-mRNA classifier for distinguishing NASH patients with fibrosis stage F2 or higher from NAFL patients and from NASH patients with fibrosis <F2.

### DETAILED DESCRIPTION

Circulating cell free-messenger RNA (cf-mRNA) monitoring can be used for blood based liver disease diagnosis to elucidate diverse biological settings. cf-mRNA can exhibit rapid transcriptional alterations associated with liver disease state and provide insight into underlying molecular liver disease mechanisms. To gain perspective on the biology and diagnosis of stages of NAFLD, whole transcriptome circulating-free messenger RNA (cf-mRNA) expression analysis can be performed in clinically characterized NAFLD patient cohorts, employing an in-house developed NGS (Next-generation Sequencing) assay. For these studies, 369 subjects from 3 patient cohorts and 303 subjects from 2 patient cohorts were tested to demonstrate the ability to diagnose NAFL and NASH liver disease states and stratify liver disease by fibrosis stages. Furthermore, data indicates that NAFLD progression may be regulated by pathways involved in hepatic stellate cell activation, FXR/RXR signaling, inflammation, liver specific pathways involved in metabolism of glucose, triglycerides, cholesterol, etc endothelial blood vessel development and adaptive immunity. Disclosed herein are systems and methods that can utilize a whole-transcriptome cf-mRNA assay to diagnose and stratify NAFLD and shed light on the liver disease mechanism.

Disclosed herein are systems and methods that can utilize a whole-transcriptome cf-mRNA assay to diagnose NAFLD and its stages and stratify NAFLD patients by liver fibrosis staging and shed light on liver disease mechanisms. Liver fibrosis can be divided into five stages: no observable fibrosis (F0), portal fibrosis without septa (F1), portal fibrosis with few septa (F2), bridging septa between central and portal veins (F3), and cirrhosis (F4).

The systems and methods described herein can use an assay that utilizes measurement of cf-mRNA directly from a sample. The sample can be saliva, blood, sputum, urine, semen, transvaginal fluid, sweat, breast milk, breast fluid, stool, a cell, or a tissue biopsy. The sample can be blood plasma. The sample can be blood serum.

The methods disclosed herein can have low sample failure rates and excellent performance across multiple patient cohorts with diverse characteristics, such as duration of serum storage (stored at −80° C. for up to 9 years), levels of hemolysis, and cellular contamination.

Methods, systems and kits described herein may relate to the rapid, noninvasive detection liver disease stages or conditions in a subject using a combination of marker types so as to concurrently determine a likely stage of liver disease, taking into account changes in gene expression brought about by angiogenesis and molecular processes involved in wound healing, activation of hepatic stellate cells, liver fibrosis, inflammation, liver specific metabolic pathways such as FXR/RXR signaling, LXR/RXR, acute phase response, PI3K/AKT signaling and neovascularization. In some embodiment, a classifier comprising a gene panel comprising genes known to be upregulated in fibrosis related to angiogenesis and endothelial blood vessel development can be applied to a cf-RNA expression profile of the subject. Through practice of the disclosure herein, one may be able to make confident predictions as to a liver disease identity and the extent of its impact on one or more tissues, without requiring any invasive investigation of the tissue or tissues suspected of being impacted.

The methods described herein can be performed with the use of a classifier. The classifier can comprise a gene panel (gene panel is used interchangeably herein with panel of genes). The classifier can define the gene panel after being trained by samples from clinically validated samples from subjects with a known stage of liver disease. The samples can be from subjects clinically validated as having a liver disease stage of: no observable fibrosis (F0), portal fibrosis without septa (F1), portal fibrosis with few septa (F2), bridging septa between central and portal veins (F3), and cirrhosis (F4). The gene panel may include one or more of UGT3B10, KNG1, HRG, CFHR3, ANGPTL3, MTTP, HPD, RTP3, FGG, FGA, FGB, APOC4-APOC2, CFHR1, SERPINA3, RP11-400G3.5, RP4-608O15.3, UGT2B15, CFHR2, ANG, SPP2, HAMP, LECT2, SERPINA6, CPB2, CYP8B1, APCS, C8A, RBP4, IGSF23, SLCO1B3, HABP2, ZNF865, C9, AADAC, FNDC5, SERPINC1, APOA2, F9, ORM2, APOB, CYP2C9, SAA4, INS-IGF2, G6PC, AHSG, THRSP, AFM, SERPIND1, HSD11B1, AMBP, BCAS3, CR1, TRMT112, KIZ, HAUS3, TMEM64, DDX3X, MYL4, PPP3CA, TPD52, CDR1-AS, CRYBG3, WDR81, EIF4G1, AQP3, APOA1, INSR, PLPP3, NAA20, HP, HECTD4, ST6GALNAC4, TUSC2, TRDMT1, KIFAP3, ADAM19, CALM2, RBFOX2, NR1D1, PDE4A, SLC25A38, NDFIP1, ZC3HAV1, AQP1, CELF2, CLSPN, ZNF333, SMU1, LY86, TOX, ALB, RNF123, ALAS2, CCNI, ZMAT3, MAP4K4, ZCCHC3, PER1, SLFN11, BET1L, APOH, XBP1, DHX38, ETFB, GCOM1, HSPG2, SAMD4A, CSTB, VAT1, VAMP3, POLD4, USP31, SLFN14, ALDOB, FAM195B, DDX39A, CUL4A, FN1, SEPN1, APOB, MT2A, EIF2D, NAP1L4, DRG1, KLHL5, SGK1, RPS13, NDUFB1, GRB10, LBR, MRPL41, PTBP3, SDHC, ALOX5, ARHGAP35, REV3L, VWF, HIST1H4I, TNS2, UZCRQ, DNASEIL3, NCL, RAB11B, SGTA, CDC37, PRR14L, ZFAND6, FGL2, OAS2, AKR1A1, PGK1, CCDC50, POLR2C, MLF2, ALDH2, RABIF, MCFD2, B3GNT8, AAK1, BAK1, GCA, BTBD9, SAFB2, KIFC3, PRDX6, LRRC4, ZNF426, VASH1, PDE8A, KIZ, HBA2, ZCCHC9, AHNAK, PRMT7, STT3A, FAM213A, NUDT9, TPGS2, SELPLG, DHRS13, MACF1, TBC1D22B, RIOK3, MOSPD3, MET, PNPO, TYK2, IKZF3, SHQ1, PRP4, C16orf62, AKAP13, UBE2Z, SLC15A3, DCAF12, SERPINB9, CDK4, KNG1, TNFAIP8L1, E2F1, CDC42EP1, INMT, NT5DC2, FSCN1, EVA1B, MLKL, ZNF462, DRAM1, TRIB3, LZTR1, EPB41L4A, RNF25, FAM127B, ZNF438, ACAD9, RASAL2, ANKRD55, WBP5, KCTD13, CD33, FMNL2, RP11-400F19.6, GRAMD4, PLCB3, GALNT10, KALRN, CTTNBP2NL, ING5, MYO10, NOVA2, AGPAT5, IFFO1, ZHX3, FRMD3, HYAL2, C8orf4, ANKRD46, GNA12, CREB3L2, ZNF561, TOR1AIP1, FEZ1, PSMB5, SEH1L, NCKAP5L, MLLT4, RBPMS, FAM114A1, MLLT4, FSCN1, MYO10, GNA12, RDX, FRMD3, BTBD6, MTSS1L, PLEKHA4, HECW2, TRAF3IP1, NDFIP1, ATXN1L, MTMR2, NUTF2, C16orf62, CTNNA1, PPP1R14B, ZNF362, ZNF358, PFKL, TSTA3, LIMCH1, SHANK3, RABGEF1, PDE2A, SNX8, TBC1D9, PITPNM3, METTL9, MAF, TRIO, MINK1, CKDAL1, TGM2, KIAA0355, PXK, CASKIN2, PEA15, CPOX, FBXW5, PNPLA6, SH3PXD2A, SAV1, TSC22D1, AKR1B1, ITSN1, BTBD1, ABCC1, CRHBP, ZNF366, DNASEIL3, FSCN1, TRIP10, ZN608, ACTA2, CCDC80, ADAMT21, IGFBP4, DDR2, HID1, RAPGEF3, AFAP1L1, IL33, PDE2A, GASH1, FEZ1, FERMT2, MAP1B, DLC1, KIAA1462, DPYSL3, PHLDB1, CNN3, CCND1, CDC43IP1, AMOTL2, PTRF, HECW2, MYH10, S100A16, RASIP1, ROBO4, TEAD2, PLK2, MAMA4, BCL6B, KDR, ADGRF5, ARHGEF15, FGD5, SHE, ECSCR, CALCRL, MPDZ, LDB2, APBB2, PTPRB, ARHGAP29, RAI14, TJP1, AKAP12, MYO10, WWTR1, MYO6, SASH1, and SEPT10.

After comparison against the panel of genes, the cf-mRNA expression levels can be further analyzed by being subjected to supervised or unsupervised clustering such as a non-negative matrix factorization. The functional categories of genes in the gene panel can be hepatic stellate cell activation, LXR/RXR signaling, Adult_endothelial_progenitor_cell, alternatively_activated_marcrophage, band_form_neutrophil, blast_forming_unit_erythroid, CD14-positive_cd16-negative_classical_monocyte, CD3-negative_cd4-positive_cd8-positive_double_positive_thymocyte, CD3-positive_cdr-positive_cd8-positive_double_positive_thymocyte, CD34-negative_cd41-positive_cd43_positive_megakaryocyte_cell, CD38-negative_naïve_b_cell, CD4-positive_alpha_beta_thermocyte, CD4-positive_alpha_beta_t_cell, Cd8-positive_alpha_beta_thermocyte, CD8-positive_alpha_beta_t_cell, central_memory_cd4-positive_alpha_beta_t_cell, central_memory_cd8-positive_alpha_beta_t_cell, class_switched_memory_b_cell, colony_forming_unit_erythroid, common_lymphoid_progenitor, common_myeloid_progenitor, conventional_dendric_cell, cytotoxic_cd56-dim_natural_killer_cell, effector_memory_cd4-positive_alpha_beta_t_cell, effector_memory_cd8-positive_alpha_beta_t_cell, effector_memory_cd8-positive_alpha_beta_t_cell_terminally_differentiated, Endothelial_cell_of_umbilical_vein_(proliferating), Endothelial_cell_of_umbilical_vein_(resting), erythroblast, germinal_center_b_cell, granulocyte_monocyte_progenitor_cell, hematopoietic_multipotent_progenitor_cell, hematopoietic_stem_cell, immature_conventional_dendric_cell, inflammatory_macrophage, late_basophilic_and_polychromatophilic_erythroblast, lymphocyte_of_b_lineage, macrophage, mature_conventional_dendric_cell, mature_eosinophil, mature neutrophil, megakaryocyte-erythroid_progenitor_cell, memory_b_cell, mesenchymal_stem_cell_of_the_bone_marrow, monocyte, mononuclear_cell_of_bone_marrow_naïve_b_cell, neuroplastic_plasma_cell, neutrophilic_metamyelocyte, neutrophilic_nyelocyte, osteoclast, peripheral_blood_mononuclear_cell, plasma_cell, regulatory_t_cell, and segmented_neutrophil_of_bone_marrow, unswitched_memory_b_cell.

Single markers and aggregate RNA derived from a sample can both be contemplated in various embodiments as indicators of liver disease stage or condition. Alternately, or in combination, circulating DNA, such as DNA that is differentially methylated in a liver disease stage-related manner, can be included as part or all of a liver disease stage-related marker.

Concurrently, markers indicative of a liver disease stage or condition may also be measured. There is a broad range of markers contemplated as indicative of a liver disease stage or condition, including proteins, steroids, lipids, cholesterols, or nucleic acids such as DNA or RNA. RNA such as particular transcripts encoding proteins implicated in a liver disease stage or condition can be useful, as are DNA having methylation patterns that are indicative of a liver disease stage. Often, but not always, the liver disease stage marker may also be a circulating marker that is readily obtained from, for example, a blood draw. However, alternatives such as ultrasound, CT scan, MRI or other data are contemplated as markers for some liver diseases.

By comparing the levels or identities of these markers to reference values or datasets of the stages of liver disease stage or condition (e.g., NALF, no observable fibrosis (F0), portal fibrosis without septa (F1), portal fibrosis with few septa (F2), bridging septa between central and portal veins (F3), and cirrhosis (F4), NASH), one may categorize a patient or a patient's sample as being indicative of a particular liver disease stage or condition in the patient. The reference values or datasets may vary as to the liver disease stage or condition, and can variously include data from one or more healthy individuals, one or more individuals suffering from various stages of a disorder or tissue duress, data from intermediate individuals, and/or data predicted from models. A sample can be categorized as indicative of a liver disease stage or condition when its values are individually or collectively above or below a threshold, or when they do not differ significantly from a reference data set correlated with the liver disease stage or condition, or when they do differ significantly from a reference dataset correlated with absence of the liver disease stage or condition.

For instance, methods, systems and kits described herein may be used to screen for development or progression of a liver disease stage, condition, or multiple conditions, in an at-risk population on a routine basis. This can be useful in subjects with chronic conditions, such as metabolic syndrome, NAFLD, sclerosing cholangitis, biliary obstructions, hepatocellular carcinoma, obesity, diabetes, or where one or more tissues are at risk of injury, damage or failure.

Metabolic syndrome and obesity affect a large and ever-growing percentage of the population worldwide. This population can be at a constant and relatively high risk of developing life-threatening complications, such as liver cirrhosis. Thus, this population can be at a constant risk of developing complications in an array of organs and tissues. In these cases, it may not be practical to assess subjects on a routine basis using traditional methods, such as imaging techniques and biopsies. However, methods, systems and kits, such as those described herein, can provide for rapidly detecting insult, increased risk and therapeutic effects in one or more organs in a subject, thereby providing a means to monitor subjects with chronic conditions for acute complications, liver disease stage progression, and therapeutic effects.

Methods, systems and kits described herein can provide for detecting or quantifying a panel of polynucleotides and/or markers related to molecular pathways of stages of liver disease. Gene expression may vary tremendously within a population of subjects and between populations of subjects (e.g., between different ethnic groups), and in such cases, a panel of liver disease stage specific polynucleotides and/or markers may be useful. While the expression levels of each liver disease stage-related polynucleotide and marker may not be similar, a conclusion or inference can still be made about the condition or tissue(s) of the subject if the panel is sufficiently similar or sufficiently different from an identified gene panel. In this way a panel may provide an advantage over using a single marker of a stage of liver disease or a single disease-related polynucleotide. In some instances, the methods may comprise comparing the cf-mRNA expression panel of a subject at a first time point to the cf-mRNA expression panel of the subject at a second time point. Thus, a single subject's natural genetic variations and gene expression fluctuations can be controlled for and differences between panels can be more likely due to changes in the condition or tissue(s) affected. In some instances, the panel may comprise non-polynucleotide molecules. The panel may comprise polynucleotides and other biological molecules (e.g., peptides, lipids, pathogen fragments, etc.).

Methods, kits, and systems described herein may be used to determine the likelihood or risk of the subject developing the liver disease stage or condition, the progression or severity of the liver disease or condition, or the effect of a therapy or treatment on the liver disease or condition. Kits, systems and methods disclosed herein can be sensitive and accurate enough to compare a first level of a marker or liver disease stage-related polynucleotide to a second level of the marker or liver disease stage-related nucleic acid, in order to differentiate between a risk of a condition, a progressed stage of a condition, or an improvement of a condition by a treatment. In some instances, the first level of the marker or liver disease stage-related nucleic acid may correspond to a sample from a subject at a first time point and the second level of the marker or liver disease stage-related nucleic acid may correspond to a second sample from a subject at a second time point.

Stages of liver disease and effected tissues may be assessed simultaneously using the kits, systems and methods disclosed herein. In this way, the kits, systems and methods disclosed herein may be used to assess the presence or absence of at least one condition and identify both affected and unaffected tissues. In some embodiments, methods may comprise selecting or recommending a medical action based on results produced by the methods, systems or kits disclosed herein. In some embodiments, a customized medical action can be recommended and/or taken, based on the determination. In some instances, customized medical action may comprises directly treating a diseased liver, e.g., with surgery or pharmaceutical intervention. Pharmaceutical intervention can include drugs targeting metabolism of lipids, metabolism of glucose, drug targeting metabolic inflexibility, drugs targeting fibrosis, anti-inflammatory compounds, acetyl-CoA Carboxylase inhibitor, OCA, elafibranor cenicrivaroc, vitamin-e, plioglitazoe, PPAR agonist, FXR agonist, ASK-1 inhibitor, fibroblasts growth factors, insulin sensitizer or bile acid regulator. Non-limiting examples of medical actions include performing additional tests (e.g., biopsy, imaging, surgery, etc.), treating the subject for the liver disease stage or condition, and modifying a treatment of the subject (e.g., altering the dose of a pharmaceutical composition, ceasing administration of a pharmaceutical composition, administering a different or additional pharmaceutical composition, etc.).

The systems, methods and kits disclosed herein may provide for detecting a stage of liver disease. In some instances, a subject may have a condition known to affect the health of liver tissue depending on the extent or severity of the condition. Systems, methods and kits, such as those disclosed herein, may allow for identification and targeted treatment of a stage of liver disease. For example, a system disclosed herein may provide for the analysis of markers for detecting inflammation in a subject and determining that the liver is affected by the inflammation due to the levels of circulating liver-specific RNAs and liver disease stage-specific RNAs.

The methods may further provide for identifying, or differentiating between, conditions that are causing the liver damage, such as between BMI and a known liver disease. Identifying, or differentiating between tissue changes and liver diseases, as described herein, can depend on quantifying (e.g., not merely detecting) the disease-related RNA and quantifying markers of the liver disease. By way of a non-limiting example, methods are disclosed herein for detecting liver disease in a subject, identifying a condition causing the liver disease, selecting a therapy to treat the subject and monitoring the effectiveness of the therapy. Cell-free RNA that corresponds to genes disclosed herein, for example, PITPNM3, LIMCH1, FSCN1, CCND1, or CASKIN2, can be quantified in a plasma sample of a subject. Differential expression of such RNA in the plasma sample may indicate that there is liver damage. The subject's cell free RNA expression data can then be adjusted according to a known expression profile of fibrosis stage related genes. A course of treatment or diagnosis may then be made based on the adjusted expression profile.

Liver disease presence and location in a subject can be determined at an early stage of liver disease because the systems and methods described herein can provide rapid results, are non-invasive and are inexpensive. Thus, the subject can be treated before the liver disease progresses to advanced stages that may be relatively more difficult to control or treat as compared to early stages. For example, the systems and methods disclosed herein may allow for determining an early stage of liver fibrosis before the disease progression is advanced enough to be visualized with an imaging technique, such as a CT or PET scan. In this way, the methods and systems disclosed herein may provide for focused analysis and targeted therapies, such as pharmaceutical intervention or dietary restrictions, at early stages of liver disease.

The methods and systems can provide for treating with a therapy that is suitable or optimal for the extent of tissue damage. In some instances, the methods may comprise detecting/quantifying the markers and/or disease-related polynucleotides to assess the effectiveness or toxicity of a therapy. In some instances, the therapy may be continued. In other instances, the therapy can be discontinued and/or replaced with another therapy. Regardless, due to the rapid and non-invasive nature of the methods and systems, therapeutic effects can be assessed and optimized more often relative to conventional treatment optimization.

In some aspects, the present disclosure can provide for uses of systems, samples, markers, and polynucleotides disclosed herein to determine a response to a therapy used to treat a liver disease stage or condition in a subject. In some instances, a response to a therapeutic in pre-clinical target discovery may be determined. Determining the response may comprise determining engagement of a target molecule in pre-clinical measurements. In some instances, a lead therapy during late-stage evaluation for further clinical development may be optimized. Evaluation may include the development of endpoints to set benchmarks for the relative therapeutic efficacy of the therapeutic agent. Benchmarks may include development of cf-mRNA signatures to evaluate the toxicity of therapeutic agents.

In some aspects, the present disclosure can provide for uses of systems, samples, markers, and polynucleotides disclosed herein. In some instances, disclosed herein are uses of an in vitro sample for non-invasively detecting a tissue or organ in a subject that is under duress and a liver disease stage or condition that may be the cause of the duress. In some instances, disclosed herein are uses of an ex vivo sample for non-invasively detecting a tissue or organ in a subject that is under duress and a liver disease stage or condition that may be the cause of the duress. Generally, uses disclosed herein comprise quantifying markers and polynucleotides in samples, including ex vivo samples and in vitro samples. Some uses disclosed herein may comprise comparing a quantity of a marker, a quantity of liver disease stage-related polynucleotide, and a quantity of a polynucleotide in a first sample and comparing the quantities to respective quantities in a second sample. In some instances, the first sample is from a first subject and the second sample is from a control subject (e.g., a healthy subject or subject with a condition or data obtained from subjects with a BMI encompassing the BMI of the subject). In some instances, the first sample is from a subject at a first time point and the second sample is from the same subject at a second time point. The first time point may be obtained before the subject is administered a therapy and the second time point may be obtained after the therapy. Thus, also provided herein are uses of samples, markers, disease-related polynucleotides, gene panels, classifiers, kits and systems that may be used to monitor or evaluate a condition of a subject, tissue health state of a subject, or an effect of a therapeutic agent.

The following descriptions are provided to aid the understanding of the methods, systems and kits disclosed herein. The following descriptions of terms used herein are not intended to be limiting definitions of these terms. These terms are further described and exemplified throughout the present application.

Methods, systems and kits described herein generally detect and quantify cell-free nucleic acids. For this reason, biological samples described herein are generally acellular biological fluids. Samples from subjects, by way of non-limiting example, may be blood from which cells are removed, plasma, serum, urine, or spinal fluid. For instance, the biological molecule may be circulating in the bloodstream of the subject, and therefore the detection reagent may be used to detect or quantify the marker in a blood or serum sample from the subject. The terms “plasma” and “serum” are used interchangeably herein, unless otherwise noted. However, in some cases they are included in a single list of sample species to indicate that both are covered by the description or claim.

The term “disease stage-related polynucleotide,” as used herein generally refers to a polynucleotide that is predominantly expressed in association with a stage of disease. Contemplated herein are polynucleotides that are predominantly expressed in association with a stage of a disease, such a liver disease, heart disease, etc. Often, methods, systems and kits disclosed herein utilize cell-free, disease-related polynucleotides. Cell-free, liver disease stage-related polynucleotides described herein are polynucleotides expressed at levels that can be quantified in a biological fluid upon damage to liver tissue. In some cases, the presence of cell-free liver disease stage-related polynucleotides disclosed herein in a biological fluid is due to release of cell-free liver disease stage-related polynucleotides upon damage of the liver and not due to a change in expression of the cell-free liver disease stage-related polynucleotides. Elevated levels of cell-free liver disease stage-related polynucleotides disclosed herein may be indicative of damage to the liver. In some instances, cell-free polynucleotides disclosed herein may be expressed/produced in several tissues, but at liver disease stage-related levels, as defined herein, in at least one of those tissues. In some instance, the cell-free polynucleotides disclosed herein may be a liver-specific transcript such as MASP2, C8A, C8B, A NGPTL3, APCS, CRP, APOA2, NR1I3, FMO3, SERPINC1, CFHR1, CFHR2, C4BPB, C4BPA, GCKR, PROC, CPS1, SPP2, AGXT, CYP8B1, RTP3, SLC38A3, ITIH1, ITIH3, ITIH4, CP, TM4SF4, SLC2A2, AHSG, FETUB, HRG, KNG1, CPN2, UGT2B10, UGT2B4, GC, ALB, AFM, HSD17B13, ADH4, ADH6, ADH1A, FGB, FGA, FGG, TDO2, F11, C9, ACOT12, LEAP2, LECT2, F12, APOM, CFB, SLC22A7, SLC22A1, PLG, IGFBP1, PON1, PON3, AKR1D1, FGL1, TTPA, BAAT, AMBP, ORM1, ORM2, C5, C8G, AKR1C4, MH2, MBL2, MAT1A, RBP4, CYP2C9, CYP2C8, ABCC2, HABP2, CYP2E1, INS-IGF2, HPX, SAA4, SAA2, SAA1, F2, APOA5, APOC3, APOA1, TTC36, SLC38A4, HSD17B6, RDH16, INHBE, PAH, SDS, HPD, CPB2, ANG, SERPINA10, SERPINA6, SERPINA1, ACSM5, TAT, HP, CA5A, GLTPD2, ASGR2, ASGR1, VTN, PIPOX, G6PC, APOH, TTR, CYP2A6, CYP2B6, APOC4, APOC2, ATF5, HAO1, LBP, FTCD, SERPIND1, UPB1, or F9.

In some instances, the cell-free polynucleotides disclosed herein may be enriched in liver associated pathways, such as, for example, the pleiotropic LXR/RXR and FXR/RXR signaling pathways involved in cholesterol, triglyceride and glucose metabolism, and acute phase response reflective of liver injury and/or inflammation. Liver associated pathways can include PI3K/AKT Signaling, IGF-1 Signaling, Hepatic Fibrosis/Hepatic Stellate Cell Activation, ILK Signaling, IL-7 Signaling Pathway, IL-3 Signaling, VEGF Signaling, Protein Kinase A Signaling, EIF2 Signaling, FXR/RXR Activation, Acute Phase Response Signaling, Regulation of eIF4 and p70S6K Signaling, LXR/RXR Activation, mTOR Signaling, Complement System, Sirtuin Signaling Pathway, Coagulation System, PXR/RXR Activation, Nicotine Degradation II, Acetone Degradation I (to Methylglyoxal), Nicotine Degradation III, Melatonin Degradation I, LPS/IL-1 Mediated Inhibition of RXR Function, Folate Polyglutamylation, Bile Acid Biosynthesis, Neutral Pathway, B Cell Development, Integrin Signaling, Ephrin Receptor Signaling, Signaling by Rho Family GTPases, PPARα/RXRα, Activation, the role of NFAT in Regulation of the Immune Response, ERK/MAPK Signaling, IL-1 Signaling, the superpathway of Melatonin Degradation, PXR/RXR Activation, Nicotine Degradation II, LPS/IL-1 Mediated Inhibition of RXR Function, Bile Acid Biosynthesis, Neutral Pathway, Atherosclerosis Signaling, Oxidative Phosphorylation, IL-12 Signaling and Production in Macrophages, Integrin Signaling, Actin Cytoskeleton Signaling, Epithelial Adherens Junction Signaling, PAK Signaling, Protein Kinase A Signaling, ILK Signaling, Actin Nucleation by ARP-WASP Complex, PI3K/AKT Signaling, Leukocyte Extravasation Signaling, CXCR4 Signaling, ERK/MAPK Signaling, or IL-8 Signaling.

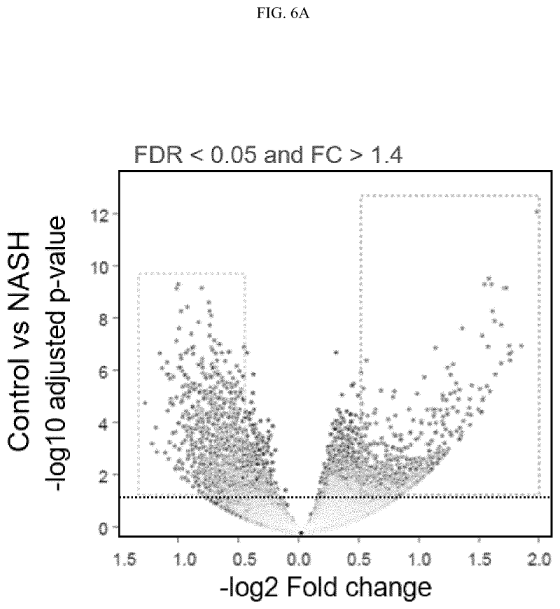

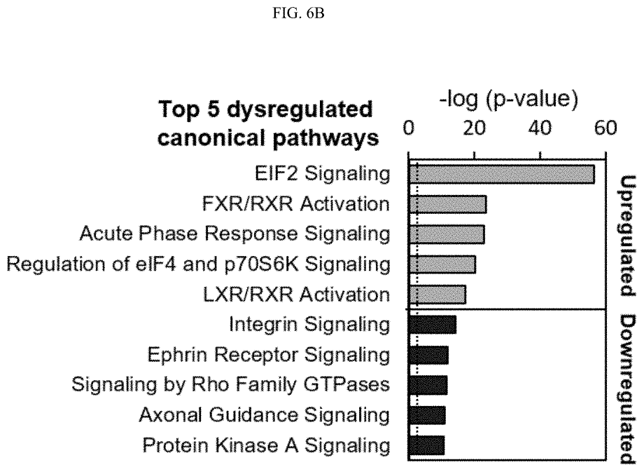

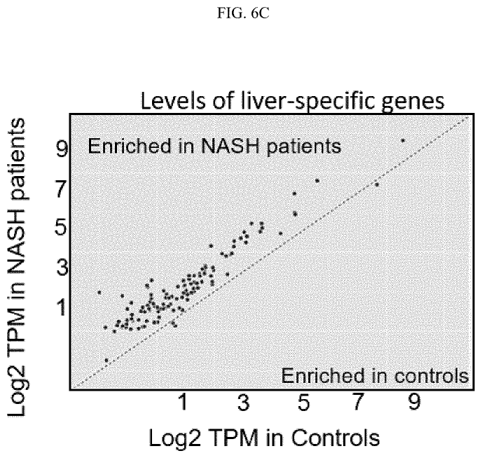

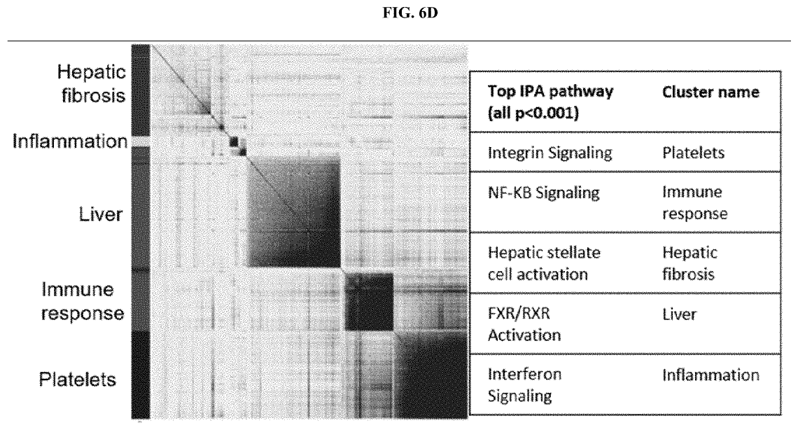

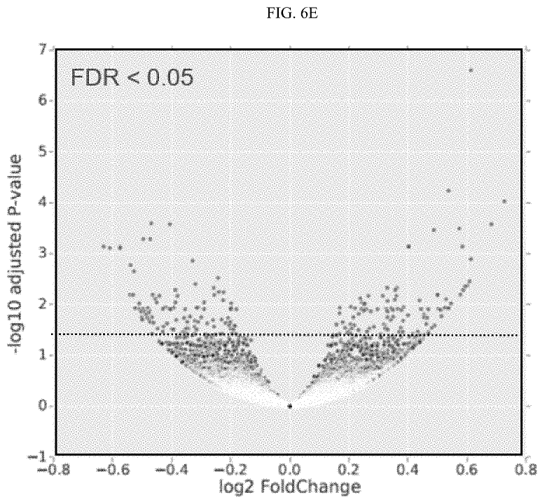

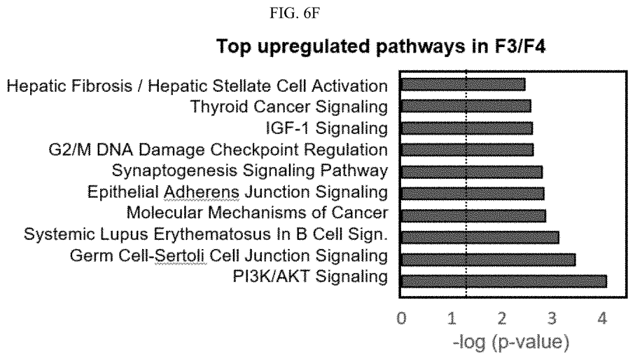

In some instances, the cell-free polynucleotides disclosed herein can originate from hepatocytes in the liver. In some instances, the cell-free polynucleotides disclosed herein can be liver-specific transcript such as those listed in Table 6. In some instances, the cell-free polynucleotides can originate from hepatic stellate cell activation (P13K/AKT signaling pathway), the central biological event of hepatic fibrosis. In some instances, the cell-free polynucleotides can originate from actin-bundling proteins. In some instances, the cell-free polynucleotides can originate from proteins responsible for the regulation of the expression of collagens and matrix metalloproteinase. In some instances, the cell-free polynucleotides can originate from inflammatory processes such as interferon signaling. In some instances, the cell-free polynucleotides can originate from canonical pathways differentially regulated between early and advanced fibrosis such as those listed in FIG. 6F. In these cases, the absolute or relative quantity of the cell-free liver disease stage-related polynucleotide can be indicative of damage to the liver, or to a collection of tissues or organs.

Alternatively, or additionally, liver disease stage-related polynucleotides may be nucleic acids with liver disease stage-related modifications. By way of non-limiting example, liver disease stage-related polynucleotides or markers disclosed herein may include DNA molecules (e.g., a portion of a gene or non-coding region) with liver disease stage-related methylation patterns. In other words, the polynucleotides and markers may be expressed similarly in many tissues, or even ubiquitously throughout a subject, but the modifications may be liver disease stage-related. Generally, liver disease stage-related polynucleotides or levels thereof disclosed herein are specific to a liver disease. Generally, liver disease stage-related polynucleotides disclosed herein encode a protein implicated in a liver disease mechanism or molecular pathway.

The term, “marker,” as used herein, generally encompasses a wide variety of biological molecules. Markers may also be referred to herein as liver disease stage markers or markers of a stage of liver disease. In some instances, the marker may be for a condition associated with a plurality of stages of liver disease. For example, the marker may be for inflammation, which can be associated with liver disease. Markers, by way of non-limiting example, include peptides, hormones, lipids, vitamins, pathogens, cell fragments, metabolites and nucleic acids. In some instances, a marker is a cell-free nucleic acid. Generally, markers disclosed herein are liver disease stage-related. However, in some instances, the markers are not liver disease stage-related. Markers disclosed herein may also be referred to as liver disease stage biomarkers. The liver disease stage biomarker can be a biological molecule that is present or produced as a result of a liver disease stage, dysregulated as a result of a liver disease stage, mechanistically implicated in a liver disease stage, mutated or modified in a liver disease stage, or any combination thereof. Markers may be produced by the subject. Markers may also be produced by other species. For instance, the marker may be a nucleic acid or protein made by a hepatitis virus or a Streptococcus bacterium. Methods for identifying such markers may further comprise detecting/quantifying disease-related polynucleotides to determine which tissues are infected or affected by these pathogens, and to an extent that the liver is damaged.

In general, the terms “cell free polynucleotide,” and “cell free nucleic acid,” used interchangeably herein, refer to a polynucleotide that can be isolated from a sample without extracting the polynucleotide from a cell. Cell free polynucleotides disclosed herein are typically polynucleotides that have been released or secreted from a damaged tissue or damaged organ, or involved with a liver-associated signaling pathway, or are involved in inflammatory processes. For example, damage to the tissue or organ may be due to a liver disease, injury or other condition that resulted in cytolysis, releasing the cell-free polynucleotide from cells of the damaged tissue into circulation. In some instances, a cell free polynucleotide disclosed herein is liver disease stage-related. In other instances, a cell free polynucleotide is not liver disease stage-related. In some instances, a cell free polynucleotide is present in a cell or in contact with a cell. In some instances, a cell free polynucleotide is in contact with an organelle, vesicle or exosome. In some instances, a cell-free polynucleotide is cell free, meaning the cell-free polynucleotide is not in contact with a cell. Cell-free polynucleotides described herein are freely circulating, unless otherwise specified. In some instances, a cell-free polynucleotide is freely circulating, that is the cell-free polynucleotide is not in contact with any vesicle, organelle or cell. In some instances, a cell-free polynucleotide is associated with a polynucleotide-binding protein (transferases, ribosomal proteins, etc.), but not any other molecules.

As used herein, the term “about” a number generally refers to that number plus or minus 10% of that number. The term “about” a range, as used herein, generally refers to that range minus 10% of its lowest value and plus 10% of its greatest value.

As used in the specification and claims, the singular forms “a,” “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement and include determining if an element is present or not (for example, detection). These terms can include quantitative, qualitative or quantitative and qualitative determinations. Assessing is alternatively relative or absolute. “Detecting the presence of,” as used herein, generally includes determining the amount of something present, as well as determining whether it is present or absent.

As used herein, the terms “treatment” or “treating” are generally used in reference to a pharmaceutical or other intervention regimen for obtaining beneficial or desired results in the recipient. Beneficial or desired results include, but are not limited to, a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect may include delaying, preventing, or eliminating the progression of a liver disease stage or condition, delaying or eliminating the onset of symptoms of a liver disease stage or condition, slowing, halting, or reversing the progression of a liver disease stage or condition, or any combination thereof. For prophylactic benefit, a subject at risk of developing a particular liver disease stage, or to a subject reporting one or more of the physiological symptoms of a liver disease stage may undergo treatment, even though a diagnosis of this liver disease stage may not have been made.

### Methods

As discussed in the foregoing and following description, methods disclosed herein may be intended to non-invasively detect a tissue or organ in a subject that is under duress as well as determine which liver disease stage or condition is affecting the liver. Some methods disclosed herein can comprise determining a stage or progress of a liver disease or condition in a subject. Some methods disclosed herein can comprise determining a response to a therapy used to treat a liver disease stage or condition in a subject. Some methods disclosed herein can comprise determining a response to a therapeutic in pre-clinical target discovery. Some methods disclosed herein can comprise determining engagement of a target molecule in pre-clinical measurements. Some methods disclosed herein can comprise optimizing a lead therapy during late-stage optimization for further clinical development. Therapy evaluation may include the development of endpoints to evaluate the relative therapeutic efficacy of the therapeutic agent. Some methods disclosed herein can comprise the development of cf-mRNA signatures to evaluate the toxicity of therapeutic agents.

Some methods disclosed herein may comprise determining if a liver in a subject is damaged, injured or infected. Some methods disclosed herein may comprise determining if a liver in a subject is affected by a liver disease stage or condition. Some methods disclosed herein may comprise detecting or quantifying a biological molecule disclosed herein. Some methods disclosed herein may comprise detecting or quantifying a marker and/or disease-related polynucleotide disclosed herein.

Some methods disclosed herein may comprise detecting a liver disease stage or condition in a subject and also detecting any tissues or organs that are under duress due to the liver disease or condition, wherein the methods may comprise comparing levels of markers and/or cell-free polynucleotides in a biological sample to threshold levels of markers and/or cell-free polynucleotides correlated with a liver-disease stage reference.

Some methods disclosed herein can comprise detecting, quantifying and/or analyzing at least one marker of a liver disease stage or condition in a sample of the subject. The methods may comprise detecting, quantifying, and/or analyzing at least one polynucleotide in a biological sample. The methods may comprise detecting, quantifying, and/or analyzing at least one liver disease stage-related polynucleotide in a biological sample. The liver disease stage-related polynucleotide may be a cell-free polynucleotide. The methods may further comprise comparing the quantity of the marker and/or the liver disease stage-related, cell-free polynucleotide to a reference level of the marker and a reference level of the liver disease stage-related polynucleotide, respectively. In some aspects, the methods can provide for the diagnosis or prognosis of the liver disease stage or condition, or assessing the progression thereof.

In some aspects, the present disclosure provides a method of determining whether a tissue has been damaged by a liver disease or condition. The method may comprise: (a) quantifying a level of or detecting at least one marker of a liver disease stage or condition in a first sample of a subject; (b) quantifying, in a second sample of the subject, a level of at least one liver disease stage-related polynucleotide, wherein the at least one liver disease stage-related polynucleotide is a cell-free polynucleotide, and further, wherein the quantifying may comprise at least one process selected from the group consisting of: reverse transcription, polynucleotide amplification, real-time PCR, sequencing, probe hybridization, microarray hybridization, and methylation-specific modification; (c) comparing the level of the at least one marker to a corresponding a reference level of the marker; (d) comparing the level of the at least one disease-related polynucleotide to a corresponding reference level of the liver disease stage-related polynucleotide; and/or (e) determining whether the tissue has been damaged by the liver disease stage or condition based on the comparing. The first sample and the second sample may be the same. The first sample and the second sample may be different. The first sample and the second sample may be obtained simultaneously. The first sample and the second sample may be obtained sequentially. By way of non-limiting example, the liver disease or condition may be selected from liver steatosis conditions (no observable fibrosis (F0), portal fibrosis without septa (F1), portal fibrosis with few septa (F2), bridging septa between central and portal veins (F3), and cirrhosis (F4)), a concurrent condition thereof, a complication thereof, a risk thereof, a stage thereof, and a response to a treatment thereof.

In another aspect, the disclosure provides a method of measuring a response to a pharmaceutical composition. The pharmaceutical composition may be a therapy in development to treat liver disease. The pharmaceutical composition may be a therapy for an indication other than liver disease wherein a relative liver toxicity requires evaluation. In some embodiments, the method may comprise: (a) quantifying a level of or detecting at least one marker of at least one liver disease stage (e.g., FSCN1, PITPNM3, LIMCH1, CCND1, or CASKIN2) in a first sample of a subject, wherein the first sample was obtained after an administration of the pharmaceutical composition; (b) quantifying in a second sample of a subject a level of at least one liver disease stage-related polynucleotide (e.g., FSCN1, PITPNM3, LIMCH1, CCND1, or CASKIN2), wherein (i) the at least one liver disease stage-related polynucleotide is a cell-free polynucleotide specific to a tissue; and (ii) the second sample was obtained after the administration of the pharmaceutical composition; (c) comparing the level of each of the at least one marker to a corresponding reference level of the marker, wherein the reference level of the marker is a level in a sample of the subject obtained prior to the administration of the pharmaceutical composition; (d) comparing the level of the at least one liver disease stage-related polynucleotide to a corresponding reference level of the liver disease stage-related polynucleotide, wherein the reference level of the liver disease stage-related polynucleotide is a level in a sample of the subject obtained prior to the administration of the pharmaceutical composition; and/or (e) determining whether the pharmaceutical composition has a therapeutic effect based on results of steps (c) and (d). The first sample and the second sample may be different. The first sample and the second sample may be obtained simultaneously. The first sample and the second sample may be obtained sequentially. By way of non-limiting example, the liver disease stage or condition may be selected from liver steatosis (NAFL, no observable fibrosis (F0), portal fibrosis without septa (F1), portal fibrosis with few septa (F2), bridging septa between central and portal veins (F3), and cirrhosis (F4), NASH), a concurrent condition thereof, a complication thereof, a risk thereof, a stage thereof, and a response to a treatment thereof.

### Treating, Monitoring, and Testing

As discussed in the foregoing and following description, methods, systems and kits disclosed herein may be intended to non-invasively detect a tissue or organ in a subject that is under duress as well as determine which liver disease or condition is affecting the tissue or organ under duress. In some instances, the methods, systems and kits can provide for treating a subject for a liver disease stage or condition. Some methods disclosed herein may comprise selecting a method or therapy for treating a subject for a liver disease stage or condition. Some kits and systems disclosed herein can provide for selecting a method or therapy for treating a subject for a liver disease stage or condition. Some methods disclosed herein can comprise monitoring a liver disease stage or condition in a subject and/or administering a test for a liver disease stage or condition. Some kits and systems disclosed herein can provide for monitoring a liver disease stage or condition in a subject and/or administering a test for a liver disease stage or condition. Some methods disclosed herein can comprise treating a subject for a liver disease stage or condition, monitoring a liver disease stage or condition in a subject, and/or administering a test for a liver disease stage or condition. In some instances, the methods disclosed herein can comprise determining the subject has a liver disease stage or condition, thereby informing the subject or their healthcare provider that a treatment or test would be appropriate, suitable, and/or beneficial to the subject. In some instances, the methods disclosed herein can comprise determining the subject has a liver disease or condition and recommending a treatment for the liver disease or condition. In some instances, the methods disclosed herein can comprise determining the subject has a liver disease or condition and treating the subject for the liver disease stage or condition. In some instances, the methods disclosed herein can comprise determining the subject has a liver disease stage or condition and monitoring the subject for the liver disease stage or condition. In some instances, the methods disclosed herein can comprise determining the subject has an increased risk or possibility of having the liver disease stage or condition relative to an individual within the same age range without the liver disease or condition and administering a test specific for the liver disease stage or condition to the subject. In some instances, the methods disclosed herein can comprise determining the subject has an increased risk or possibility of having the liver disease stage or condition relative to an individual within the same age range without the liver disease stage or condition and recommending a test specific for the liver disease stage or condition to the subject.

Provided herein are therapeutic agents, compositions, compounds and agents that may be used for the treatment of liver diseases and conditions. An “analog,” as used herein, generally refers to a modified or synthetic compound that resembles a naturally-occurring compound, wherein at least 50% of the analog structure is identical to at least 50% of the naturally-occurring compound.

Liver disease presence and location in a subject can be determined at an early stage of liver disease stage with greater accuracy, for example, because the systems and methods described herein may provide rapid results, take into account gene expression variations in the stages of liver disease, and can be non-invasive and/or inexpensive. Thus, the subject can be treated before the liver disease stage progresses to advanced stages that may be relatively more difficult to control or treat as compared to early stages. For example, the systems and methods disclosed herein may allow for determining if a subject has NAFL before progressing to NASH and determining if the patient has NASH with low fibrosis, before progressing to significant fibrosis. The systems and methods disclosed herein may allow for determining if a subject has liver disease stage F0 before progressing to liver disease stage F1. The systems and methods disclosed herein may allow for determining if a subject has liver disease stage F1 before progressing to liver disease stage F2. The systems and methods disclosed herein may allow for determining if a subject has liver disease stage F2 before progressing to liver disease stage F3. The systems and methods disclosed herein may allow for determining if a subject has liver disease stage F3 before progressing to liver disease stage F4. In this way, the methods and systems disclosed herein may provide for focused analysis and targeted therapies at early stages of liver disease (e.g., F0 and F1).

The methods and systems may provide for treating with a therapy that is suitable or optimal for the extent of tissue damage present in the individual. In some instances, the methods can comprise detecting/quantifying the markers and/or liver disease stage-related polynucleotides to assess the effectiveness and/or toxicity of a therapy. In some instances, the therapy may be continued. In other instances, the therapy may be discontinued and/or replaced with another therapy. Regardless, due to the rapid and non-invasive nature of the methods and systems, therapeutic effects can be assessed and optimized more often relative to some conventional treatment optimization.

In some aspects, the present disclosure provides for uses of systems, samples, markers, and liver disease stage-related polynucleotides disclosed herein. In some instances, disclosed herein are uses of an in vitro sample for non-invasively detecting a liver in a subject that is under duress and a liver disease stage or condition that is the cause of the duress. In some instances, disclosed herein are uses of an ex vivo sample for non-invasively detecting a liver in a subject that is under duress and a liver disease stage or condition that is the cause of the duress by comparing the gene expression data to a liver disease stage specific expression control. Generally, uses disclosed herein comprise quantifying markers and disease-related polynucleotides in samples, including ex vivo samples and in vitro samples. Some uses disclosed herein comprise comparing a quantity of a marker and a quantity of liver disease stage-related polynucleotide in a first sample and comparing the quantities to respective quantities in a second sample. In some instances, the first sample is from a first subject and the second sample is from a control subject (e.g., a healthy subject or a subject with a clinically verified stage of liver disease or a healthy subject or a subject with a clinically verified stage of liver disease, wherein the subject is in the same age range as the first subject). In some instances, the first sample may be from a subject at a first time point and the second sample may be from the same subject at a second time point. The first time point may be obtained before the subject is administered a therapy and the second time point may be obtained after the therapy. Thus, also provided herein are uses of samples, markers, disease-related polynucleotides, kits and systems to monitor and/or evaluate a condition of a subject, tissue health state of a subject, and/or an effect of a therapeutic agent.

In some aspects, the disclosure provides for methods of monitoring a human subject with a chronic condition for a presence of at least one liver disease complication of at least one tissue. In some aspects, the disclosure provide for methods of monitoring a human subject with a chronic liver condition for an increased risk of at least one complication of at least one tissue.

In some aspects, the disclosure provides for methods of monitoring a human subject with a chronic metabolic condition for a presence of at least one complication of a molecular pathway associated with a stage of liver disease. In some aspects, the disclosure provide for methods of monitoring a human subject with a chronic metabolic condition for an increased risk of at least one complication of a molecular pathway associated with a stage of liver disease.

Some methods comprise monitoring the human subject for a complication related to a stage of liver disease in any one of at least three tissues. Some methods comprise monitoring the human subject for an increased risk of a complication related to a stage of liver disease in any one of at least three tissues.

Some methods may comprise the steps of: obtaining a biological fluid from the subject; measuring a marker level in the biological fluid, wherein the marker is selected from a cholesterol, a lipid, insulin, an inflammatory mediator, a lipid mediator, an insulin mediator and a cholesterol mediator; and/or quantifying ribonucleic acids (RNA) in the biological fluid from liver, cardiovascular tissue, nervous system, and kidney. In some cases, a threshold marker level and a threshold quantity of the RNA may indicate the presence or increased risk of the liver-disease stage related complication in at least one of the cardiovascular tissue, nervous system and kidney.

As used herein, the term “chronic condition” generally refers to a condition that the subject has experienced for at least about six months. In some instances, a chronic condition may be a condition that the subject has experienced for at least about one year. In some instances, a chronic condition may be a condition that the subject has experienced for at least about six months to at least about one year. In some instances, a chronic condition may be a condition that the subject has experienced for at least about six months to at least about two years. In some instances, the chronic condition may be a chronic metabolic condition. In some instances, the chronic condition may be obesity. In some instances, the chronic condition may be alcoholism. In some instances, the chronic condition may be addiction to a substance that can cause liver damage.

As used herein, the term “complication” generally includes a condition that is acute, a condition that is life-threatening, a condition that requires immediate intervention, a condition that warrants immediate attention, a condition of which immediate attention or intervention would prevent a life-threatening incident, and combinations thereof. Non-limiting examples of liver-disease stage related complications are renal ischemia, renal failure, liver failure, liver cirrhosis, liver fibrosis, non-alcoholic steatohepatitis, viral hepatitis, arterial thrombosis, arterial occlusion, valvular heart liver disease, atherosclerotic plaques, aneurysm, peripheral artery liver disease, blood clot, pericarditis, and cardiomyopathy.

In some instances, an increased risk of at least one liver-disease stage related complication may be a substantially greater risk in the subject relative to a risk of the at least one complication in a subject that does not have a stage of liver disease. In some instances, an increased risk of at least one complication may be a substantially greater risk in a first subject that has the stage of liver disease relative to a risk of the at least one complication in a second subject that does not have the stage of liver disease.

Gene expression panels as disclosed herein share a property that sensitive, specific conclusions regarding an individual's tissue liver disease stage can be made using cfRNA expression level information derived from circulating blood. A benefit of the present gene marker panels may be that they provide a sensitive, specific, liver health assessment using conveniently, noninvasively obtained samples. There is no need to rely upon additional data obtained from intrusive biopsies. As a result, compliance rates may be substantially higher and liver health issues may be more easily recognized early in their progression so that they may be more efficiently treated.

Gene marker panels as disclosed herein may be selected such that their predictive value is substantially greater than the predictive value of their individual members or the expression values measured from an individual alone. Panel members may co-vary with one another. Panel members may not co-vary with one another. Panel members which do not co-vary may provide independent contributions to the panel's overall health signal.

Accordingly, a panel may be able to substantially outperform the performance of any individual constituent indicative of an individual's tissue health status such that a commercially and medicinally relevant degree of confidences (sensitivity and/or specificity) can be obtained.

### Isolating, Quantifying, and Detecting

Methods disclosed herein may comprise detecting or quantifying an amount of a marker of a liver disease stage or condition disclosed herein in to determine that the subject is affected by a respective liver disease stage or condition or that the subject is at a risk of being affected by a respective liver disease stage or condition. In some instances, detecting or quantifying at least 1 copy/ml of the marker can be sufficient to determine that the subject is affected by, or at risk of being affected by, a respective liver disease stage or condition. In some instances, detecting or quantifying at least 5 copies/ml of the marker can be sufficient to determine that the subject is affected by, or at risk of being affected by, a respective liver disease stage or condition. In some instances, detecting or quantifying at least 10 copies/ml of the marker can be sufficient to determine that the subject is affected by, or at risk of being affected by, a respective liver disease stage or condition. In some instances, detecting or quantifying at least 15 copies/ml of the marker can be sufficient to determine that the subject is affected by, or at risk of being affected by, a respective liver disease stage or condition. In some instances, detecting or quantifying at least 20 copies/ml of the marker can be sufficient to determine that the subject is affected by, or at risk of being affected by, a respective liver disease stage or condition. In some instances, detecting or quantifying at least 25 copies/ml of the marker can be sufficient to determine that the subject is affected by, or at risk of being affected by, a respective liver disease stage or condition. In some instances, detecting or quantifying at least 30 copies/ml of the marker can be sufficient to determine that the subject is affected by, or at risk of being affected by, a respective liver disease stage or condition. In some instances, detecting or quantifying at least 40 copies/ml of the marker can be sufficient to determine that the subject is affected by, or at risk of being affected by, a respective liver disease stage or condition. In some instances, detecting or quantifying at least 50 copies/ml of the marker can be sufficient to determine that the subject is affected by, or at risk of being affected by, a respective liver disease stage or condition. In some instances, detecting or quantifying at least 100 copies/ml of the marker can be sufficient to determine that the subject is affected by, or at risk of being affected by, a respective liver disease stage or condition.

Furthermore, methods disclosed herein can comprise detecting or quantifying an amount of a liver disease stage-related polynucleotide disclosed herein in to determine that a liver tissue is being affected by a liver disease stage or condition. In some instances, methods can comprise detecting or quantifying at least 1 copy/ml of the liver disease stage-related polynucleotide. In some instances, methods can comprise detecting or quantifying at least 5 copies/ml of the liver disease stage-related polynucleotide. In some instances, methods can comprise detecting or quantifying at least 10 copies/ml of the liver disease stage-related polynucleotide. In some instances, methods can comprise detecting or quantifying at least 15 copies/ml of the liver disease stage-related polynucleotide. In some instances, methods can comprise detecting or quantifying at least 20 copies/ml of the liver disease stage-related polynucleotide. In some instances, methods can comprise detecting or quantifying at least 25 copies/ml of the liver disease stage-related polynucleotide. In some instances, methods can comprise detecting or quantifying at least 30 copies/ml of the liver disease stage-related polynucleotide. In some instances, methods can comprise detecting or quantifying at least 35 copies/ml of the liver disease stage-related polynucleotide. In some instances, methods can comprise detecting or quantifying at least 40 copies/ml of the liver disease stage-related polynucleotide. In some instances, methods can comprise detecting or quantifying at least 45 copies/ml of the liver disease stage-related polynucleotide. In some instances, methods can comprise detecting or quantifying at least 50 copies/ml of the liver disease stage-related polynucleotide. In some instances, methods can comprise detecting or quantifying at least 100 copies/ml of the liver disease stage-related polynucleotide.

Some methods disclosed herein may comprise detecting or quantifying at least a certain amount of a marker or liver disease stage-related polynucleotide in order to determine that a liver disease stage or condition is affecting a respective tissue. In some cases, the amount of the marker, wherein the marker is a polynucleotide, or liver disease stage-related polynucleotide may be at least about 1 copy/mL, at least about 10 copies/mL, at least about 20 copies/mL, at least about 30 copies/mL, at least about 40 copies/mL, or at least about 50 copies/mL, at least about 80 copies/cell, at least about 100 copies/cell, at least about 120 copies/cell, at least about 150 copies/cell, or at least about 200 copies/cell. In some cases, the amount of the marker, wherein the marker is a protein, lipid, or other non-polynucleotide biological molecule, may be at least about 5 pg/mL, at least about 10 pg/mL, at least about 20 pg/mL, at least about 30 pg/mL, at least about 50 pg/mL, at least about 60 pg/mL, at least about 80 pg/mL, at least about 100 pg/mL, at least about 150 pg/mL, at least about 200 pg/mL, or at least about 500 pg/mL.

As discussed in the foregoing and following description, methods and systems disclosed herein can be intended to non-invasively detect a liver under duress as well as determine which liver disease stage or condition is affecting the tissue or organ under duress by detecting, quantifying, or otherwise analyzing at least one marker and at least one liver disease stage-related polynucleotide disclosed herein. In some cases, the at least one marker can comprise a polynucleotide (e.g., cell-free polynucleotide) or a polypeptide. Some methods may comprise detecting the polynucleotide or polypeptide by contacting the polynucleotide or polypeptide with at least one probe. In some cases, the at least one probe may only be capable of binding to a wildtype version of the polynucleotide or polypeptide. In some cases, the at least one probe may only be capable of binding to a mutant version of the polynucleotide or polypeptide. In some cases, wherein the marker is a polynucleotide, detection may comprise sequencing.

Some methods disclosed herein may comprise isolating at least one marker and/or at least one liver disease stage-related polynucleotide. In some cases, the at least one marker and/or at least one liver disease stage-related polynucleotide may comprise a cell-free polynucleotide. In some cases, isolating the cell-free polynucleotide may comprise fractionating the sample from the subject. Some methods may comprise removing intact cells from the sample. For example, some methods may comprise centrifuging a blood sample and collecting the supernatant that is serum or plasma, or filtering the sample to remove cells. In some embodiments, cell-free polynucleotides can be analyzed without fractionating the sample from the subject. For example, urine, cerebrospinal fluid or other fluids that contain little to no cells may not require fractionating. Some methods may comprise sufficiently purifying the cell-free polynucleotides in order to detect/quantify/analyze the cell-free polynucleotides. Various reagents, methods and kits can be used to purify the cell-free polynucleotides. Reagents include, but are not limited to, phenol, phenol-chloroform, glycogen, sodium iodide, detergents and chaotropic salts. Kits include, but are not limited to, Thermo Fisher ChargeSwitch® Serum Kit, Qiagen RNeasy Kit, ZR serum DNA kit, Puregene DNA purification system, QIAamp DNA Blood Midi kit, miRNeasy, exoRNeasy, QIAamp Circulating Nucleic Acid Kit, and QIAamp ccfDNA/RNA kit.

Some methods disclosed herein may comprise enriching a sample for cell-free polynucleotides. For example, a sample of interest may contain RNA/DNA from bacteria. Some methods may comprise exosomal capture, thereby eliminating, or substantially eliminating, unwanted sequences and enriching the sample for polynucleotides of interest. In some cases, exosomal capture may comprise array-based capture or in-solution capture, fragments of DNA corresponding to RNAs of interest tethered to a surface or beads, respectively. Some methods may also comprise filtering or removing other biological molecules or cells from the sample, such as proteins or platelets. In some instances, enriching the sample for cell-free polynucleotides can include preventing blood cell RNA contamination of a plasma sample. In some instances, using tubes free of EDTA can prevent or reduce the presence of blood cell RNA in a plasma/serum sample.

Generally, methods disclosed herein may comprise detecting or quantifying at least one marker and/or at least one liver disease stage-related polynucleotide. In some instances, quantifying and/or detecting the at least one marker and/or at least one liver disease stage-related polynucleotide may comprise amplifying the at least one marker and/or at least one liver disease stage-related polynucleotide. In some cases involving cell-free RNA, quantifying and/or detecting the at least one marker and/or at least one disease-related polynucleotide may comprise reverse transcribing the cell-free RNA. Any of a variety of processes can be employed to detect/quantify the marker or liver disease stage-related polynucleotide in a sample. In some cases involving cell-free, liver disease stage-related RNAs, RNA can be isolated from a sample and reverse transcribed to produce cDNA prior to further manipulation, such as amplification and/or sequencing. In some embodiments, amplification can be initiated at the 3′ end as well as randomly throughout the whole transcriptome in the sample to allow for amplification of both mRNA and non-polyadenylated transcripts. Suitable kits for amplifying cDNA include, for example, the Ovation® RNA-Seq System. Liver disease stage-related RNAs can be identified and quantified by a variety of techniques, such as, but not limited to, array hybridization, quantitative PCR, ddPCR and sequencing.

Some methods disclosed herein can comprise quantifying at least one marker and/or at least one disease-related polynucleotide described herein. In some cases, quantifying can be useful for determining the stage of liver disease. For example, some methods may comprise comparing a quantity of marker and/or liver disease stage-related polynucleotide to a quantity of marker and/or liver disease stage-related polynucleotide in a first sample at a first time in the subject and quantifying the marker and/or liver disease stage-related polynucleotide in a second sample at a second time, wherein the subject was subjected to a therapy between the first time and the second time. Some methods may comprise maintaining the therapy or changing the therapy (e.g., type, dose, etc.) based on information that resulted from the quantifying. Some methods can comprise quantifying the marker and/or disease-related polynucleotide in additional samples at additional times, in between which the therapy is modulated.

Some methods of quantifying nucleic acids disclosed herein can comprise sequencing at least one nucleic acid. Sequencing may be targeted sequencing. In some cases, targeted sequencing may comprise specifically amplifying a select marker or a select liver disease stage-related polynucleotide as disclosed herein (e.g., PITPNM3, LIMCH1, FSCN1, CCND1, or CASKIN2) and sequencing the amplification products. In some cases, targeted sequencing may comprise specifically amplifying a subset of selected markers or a subset of select liver disease stage-related polynucleotides disclosed herein and sequencing the amplification products. Alternatively, some methods comprising targeting sequencing may not comprise amplifying the markers or liver disease stage-related polynucleotides. Some methods can comprise untargeted sequencing. In some instances, untargeted sequencing may comprise sequencing the amplification products, wherein a portion of the cell-free nucleic acids are not markers or liver disease stage-related polynucleotides. In some instances, untargeted sequencing may comprise amplifying cell-free nucleic acids in a sample from the subject and sequencing the amplification products, wherein a portion of the cell-free nucleic acids are not markers or liver disease stage-related polynucleotides. In some instances, untargeted sequencing may comprise amplifying cell-free nucleic acids comprising a marker or liver disease stage-related polynucleotide described herein. Sequencing may provide a number of reads that corresponds to a relative quantity of the marker or liver disease stage-related polynucleotide. In some instances, sequencing may provide a number of reads that corresponds to an absolute quantity of the marker or liver disease stage-related polynucleotide. In some embodiments, the amplified cDNA may be sequenced by whole transcriptome shotgun sequencing (also referred to as “RNA-Seq”). Whole transcriptome shotgun sequencing (RNA-Seq) can be accomplished using Sanger sequencing, sequencing by synthesis, pyrosequencing, sequencing using nanopores, high throughput sequencing techniques, or a variety of next-generation sequencing platforms such as, but not limited to the Illumina Genome Analyzer platform, ABI Solid Sequencing platform, or Life Science's 454 Sequencing platform.

In some instances, identification of specific targets can be performed by microarray, such as a peptide array or oligonucleotide array, in which an array of addressable binding elements specifically bind to corresponding targets, and a signal proportional to the degree of binding is used to determine quantity of the target in the sample. In some cases, sequencing may be a method of quantifying. In some instances, sequencing can allow for parallel interrogation of thousands of genes without amplicon interference. In some instances, quantifying by sequencing is used instead of quantifying by Q-PCR. In some instances, for example, there are so many control genes required to accurately quantify gene expression by Q-PCR, that quantifying with Q-PCR may be inefficient. In other instances, sequencing efficiency and accurate quantification by sequencing may not be affected by the number of genes (e.g., control genes) analyzed. For at least the foregoing reasons, sequencing can be useful for some methods disclosed herein, wherein the health status of multiple organs (e.g., heart, kidney, liver, etc.) is assessed.

Some methods of quantifying a nucleic acid disclosed herein may comprise quantitative PCR (q-PCR). In some instances, Q-PCR may comprise a reverse transcription reaction of cell-free RNAs described herein to produce corresponding cDNAs. In some instances, cell-free RNA may comprise a marker, a liver disease stage-related polynucleotide, and/or a cell-free RNA that is neither a marker nor a liver tissue specific polynucleotide. Some cell-free RNA may comprise a marker described herein, a liver disease stage-related polynucleotide described herein, and/or a cell-free RNA that is neither a marker nor a liver tissue specific polynucleotide described herein. In some cases, Q-PCR may comprise contacting the cDNAs that correspond to a marker, a liver disease stage-related polynucleotide, or a housekeeping gene (e.g., ACTB, GAPDH) with PCR primers specific to the marker, disease-related polynucleotide, or housekeeping gene.

Some methods disclosed herein comprise quantifying a “housekeeping” polynucleotide. Methods comprising Q-PCR disclosed herein may comprise contacting nucleic acids with primers corresponding to a blood cell-specific polynucleotide. Some blood cell-specific polynucleotides disclosed herein can be nucleic acids that are predominantly expressed or even exclusively expressed by one or more types of blood cells. Types of blood cells can be generally categorized as white blood cells (also referred to as leukocytes), red blood cells (also referred to as erythrocytes), and platelets. In some instances, the blood cell-specific polynucleotide may also be used as a control in methods comprising quantifying disease-related polynucleotides and liver disease markers disclosed herein. In some cases, absence of an amplification product with primers corresponding to a blood cell-specific polynucleotide may be used to confirm the method is detecting cell-free RNAs in a blood, plasma or serum sample and not RNA expressed in blood cells. By way of non-limiting example, blood-cell specific polynucleotides include polynucleotides expressed in white blood cells, platelets or red blood cells, and combinations thereof. White blood cells include, but are not limited to, lymphocytes, T-cells, B cells, dendritic cells, granulocytes, monocytes, and macrophages. By way of non-limiting example, the blood-specific polynucleotide may be encoded by a gene selected from CD4, TMSB4X, MPO, SOX6, HBA1, HBA2, HBB, DEFA4, GP1BA, CD19, AHSP, and/or ALAS2. The blood cell-specific polynucleotide may be encoded by CD4 and predominantly expressed by white blood cells. The blood cell-specific polynucleotide may be encoded by TMSB4X and expressed by multiple blood cell types (whole blood). The blood cell-specific polynucleotide may be encoded by MPO and predominantly expressed by neutrophil granulocytes. The blood cell-specific polynucleotide may be encoded by DEFA4 and predominantly expressed by neutrophils. The blood cell-specific polynucleotide may be encoded by GP1BA and predominantly expressed by platelets. The blood cell-specific polynucleotide may be encoded by CD19 and predominantly expressed by B cells. The blood cell-specific polynucleotide may be encoded by ALAS2, SOX6, HBA1, HBA2, or HBB and predominantly expressed by erythrocytes.

In some cases, ddPCR or Q-PCR is a method of quantifying. Q-PCR may be a more sensitive method and therefore more accurately quantify RNA present at very low levels. In some instances, quantifying by Q-PCR is used instead of quantifying by sequencing. In some instances, sequencing may require more complex preparation of RNA samples and may require depletion or enrichment of nucleic acids in order to provide accurate quantification.

Often, methods disclosed herein can comprise detecting or quantifying a combination of markers or a combination of liver disease stage-related polynucleotides. In some cases, a more conclusory diagnosis or assessment of the subject can be performed if multiple liver-specific, fibrotic or inflammation related polynucleotides are detected. In some cases, the presence of each of the liver-specific or inflammation related polynucleotides in a blood sample of the subject would not be indicative of damage to the liver. However, their presence may collectively indicate damage to the liver. Similarly, a more conclusory diagnosis or assessment of the subject can be performed if multiple markers are detected. In some cases, the presence of each of the markers in a blood sample of the subject would not be indicative of damage to the liver. However, their presence may collectively indicate the condition in the liver. The methods may comprise detecting or quantifying about 2, about 3, about 4, about 5, about 6, about 7, about 8, about 9, or about 10 liver-specific or inflammation related polynucleotides. The methods may comprise detecting or quantifying about 2, about 3, about 4, about 5, about 6, about 7, about 8, about 9, or about 10 markers. Two or more of the markers may be known to interact in a common genetic pathway or common molecular signaling pathway. The common molecular signaling pathway may be a network of several proteins interacting to enact a cellular function, such as, by way of non-limiting example, an inflammatory response, hepatic stellate cell activation, apoptosis, cholesterol uptake, etc.

Similarly, in the case of cell-free DNAs, some methods disclosed herein may employ liver disease stage-related modifications of DNA or chromatin to identify the disease-related polynucleotide in the sample. For example, a liver disease stage-related cell-free DNA may comprise a liver disease stage-related methylation pattern. A liver disease stage-related cell-free DNA may be complexed with a protein that is indicative of a specific tissue of origin (e.g., a transcription factor known to transcribe the gene in a particular tissue). Cell-free or circulating chromatin or chromatin fragments may have liver disease stage-related histone modifications (e.g., methylation, acetylation, and phosphorylation). In some of these cases, a method such as chromatin immunoprecipitation may be suitable for detecting/quantifying the disease-related polynucleotide. Cell-free liver disease stage-related DNA may be single-stranded or double-stranded DNA.

Some methods disclosed herein may comprise use of a variety of methods of detecting the methylation pattern. The DNA can be subjected to a chemical conversion process that selectively modifies either methylated or unmethylated nucleotides. For example, the DNA may be treated with bisulfite, which converts cytosine residues to uracil (which are converted to thymidine following PCR) but leaves 5-methylcytosine residues unaffected. Thus, bisulfite treatment introduces specific changes in the DNA sequence that depend on the methylation status of individual cytosine residues (“methylation-specific modification”), yielding single-nucleotide resolution information about the methylation status of a segment of DNA. Various analyses can be performed on the altered sequence to retrieve this information. Other methods of detecting the methylation pattern are also within the scope of this disclosure.

Some methods disclosed herein can comprise subjecting DNA to oxidizing or reducing conditions prior to bisulfite treatment, so as to identify patterns of other epigenetic marks. For example, an oxidative bisulfite reaction can be performed. 5-methylcytosine and 5-hydroxymethylcytosine both read as a C in bisulfite sequencing. An oxidative bisulfite reaction can allow for the discrimination between 5-methylcytosine and 5-hydroxymethylcytosine at single base resolution. Typically, the method can employ a specific chemical oxidation of 5-hydroxymethylcytosine to 5-formylcytosine, which subsequently converts to uracil during bisulfite treatment. The only base that then reads as a C is 5-methylcytosine, giving a map of the true methylation status in the DNA sample. Levels of 5-hydroxymethylcytosine can also be quantified by measuring the difference between bisulfite and oxidative bisulfite sequencing. DNA may also be subjected to reducing conditions prior to bisulfite treatment. Reduction converts 5-formylcytosine residues in the sample nucleotide sequence into 5-hydroxymethylcytosine. As noted above, 5-formylcytosine converts to uracil upon bisulfite treatment, but 5-hydroxymethylcytosine does not. By comparing a first portion of a sample subjected to reductive bisulfite treatment to a second portion of a sample subjected to bisulfite treatment alone, locations of 5-formylcytosine marks can be identified.

As an alternative to inducing sequence changes based on methylation, methods disclosed herein may comprise inferring methylation status may by isolating or enriching polynucleotides comprising methylation and identifying the methylated polynucleotides based on their sequences (e.g., by sequencing or probe hybridization). One process for enriching methylated sequences may comprise modifying bases in a methylation-specific fashion, enriching for polynucleotides comprising the modification (e.g., by purification), amplifying the enriched polynucleotides, and/or then identifying the polynucleotides. For example, 5-hydroxymethyl-modified cytosines (5hmC) may be selectively glycosylated in the presence of a UDP-glucose molecules and a beta-glucosyltransferase. The UDP-glucose molecules may comprise a label, such that the label becomes conjugated to the 5hmC-containing polynucleotide upon reaction with the UDP-glucose. The label can be a member of a binding pair (e.g., streptavidin/biotin or antigen/antibody), which allows isolation of modified fragments upon binding to the corresponding member of the binding pair. Isolated polynucleotides may be further enriched, such as in an amplification reaction (e.g., PCR), prior to identification.

Presence and/or quantity (relative or absolute) of a polynucleotide, as well as changes in sequence resulting from bisulfite treatment, can be detected using any suitable sequence detection method. Examples include, but are not limited to, probe hybridization, primer-directed amplification, and sequencing. Polynucleotides may be sequenced using any convenient low- or high-throughput sequencing technique or platform, including, but not limited to, Sanger sequencing, Solexa-Illumina sequencing, Ligation-based sequencing (SOLiD), pyrosequencing; strobe sequencing (SMR); and semiconductor array sequencing (Ion Torrent). The Illumina or Solexa sequencing can be based on reversible dye-terminators. DNA molecules are typically attached to primers on a slide and amplified so that local clonal colonies are formed. Subsequently, one type of nucleotide at a time may be added, and non-incorporated nucleotides are washed away. Subsequently, images of the fluorescently labeled nucleotides may be taken and the dye is chemically removed from the DNA, allowing a next cycle. The Applied Biosystems' SOLiD technology employs sequencing by ligation. This method is based on the use of a pool of all possible oligonucleotides of a fixed length, which are labeled according to the sequenced position. Such oligonucleotides are annealed and ligated. Subsequently, the preferential ligation by DNA ligase for matching sequences typically results in a signal informative of the nucleotide at that position. Since the DNA is typically amplified by emulsion PCR, the resulting bead, each containing only copies of the same DNA molecule, can be deposited on a glass slide resulting in sequences of quantities and lengths comparable to Illumina sequencing.

Another example of an envisaged sequencing method is pyrosequencing, in particular 454 pyrosequencing, e.g., based on the Roche 454 Genome Sequencer. This method amplifies DNA inside water droplets in an oil solution with each droplet containing a single DNA template attached to a single primer-coated bead that then forms a clonal colony. Pyrosequencing uses luciferase to generate light for detection of the individual nucleotides added to the nascent DNA, and the combined data are used to generate sequence read-outs. A further method is based on Helicos' Heliscope technology, wherein fragments are captured by polyT oligomers tethered to an array. At each sequencing cycle, polymerase and single fluorescently labeled nucleotides are added and the array is imaged. The fluorescent tag is subsequently removed, and the cycle is repeated. Further examples of suitable sequencing techniques are sequencing by hybridization, sequencing by use of nanopores, microscopy-based sequencing techniques, microfluidic Sanger sequencing, or microchip-based sequencing methods. High-throughput sequencing platforms can permit generation of multiple different sequencing reads in a single reaction vessel, such as 10³, 10⁴, 10⁵, 10⁶, 10⁷ or more.

Some methods, systems and kits disclosed herein can provide for quantifying a liver tissue's relative contribution to a cell-free transcriptome of a biological sample. In some instances, quantifying a liver tissue's relative contribution to a cell-free transcriptome may comprise quantifying total RNA in the sample. In some instances, quantifying a liver tissue's relative contribution to a cell-free transcriptome may comprise quantifying total nucleic acids in the sample. In some instances, the relative contribution of the tissue can be compared to that of a control cell-free transcriptome in a control sample. If the relative contribution of the liver tissue is similar to that of a control cell-free transcriptome, the liver tissue can be considered to have a similar health status as that of a control liver tissue contributing to the control cell-free transcriptome. If the relative contribution of the liver tissue is different from that of a control cell-free transcriptome, the liver tissue may be considered to have a different health status than that of a control liver tissue contributing to the control cell-free transcriptome. In some cases, the control cell-free transcriptome can be representative of a healthy individual or a healthy population with the control tissue being healthy, liver disease-free, and/or liver damage-free.

Some methods and systems disclosed herein can provide for deconvolution of a cell-free transcriptome to determine the relative contribution of a subject's liver towards the cell-free RNA transcriptome. In some instances, the following steps may be employed to determine the relative RNA contributions of a subject's liver in a sample. First, a panel of disease-related transcripts can be identified. Second, total RNA in plasma from a sample can be determined. Third, the total RNA can be assessed against the panel of liver disease stage-related transcripts, and the total RNA can be considered a summation of these different liver disease stage-related transcripts. Quadratic programming can be used as a constrained optimization method to deduce the relative optimal contributions of different organs/tissues towards the cell-free transcriptome of the sample. In certain embodiments, quadratic programming may be used as a constrained optimization method to deduce relative optimal contributions of different organs/tissues towards the cell-free transcriptome in a sample. Quadratic programming is described in Goldfarb and A. Idnani (1982). Dual and Primal-Dual Methods for Solving Strictly Convex Quadratic Programs. In J. P. Hennart (ed.), Numerical Analysis, Springer-Verlag, Berlin, Pages 226-239, and D. Goldfarb and A. Idnani (1983). A numerically stable dual method for solving strictly convex quadratic programs. Mathematical Programming, 27, 1-33.

In some cases, the methods may comprise normalizing cell-free transcript values. This can involve rescaling cell-free transcript values to housekeeping gene transcript values. Next, the sample's total RNA can be assessed against the panel of disease-related genes using quadratic programming in order to determine the disease-related relative contributions to the sample's cell-free transcriptome. The following constraints can be employed to obtain the estimated relative contributions during the quadratic programming analysis: a) the RNA contributions of different tissues are greater than or equal to zero and b) the sum of all contributions to the cell-free transcriptome equals one.

Some methods, systems and kits disclosed herein can provide for determining the relative contribution of a tissue to determine a reference level for the tissue. That is, a certain population of subjects (e.g., diseased or normal) can be subject to the deconvolution process to obtain reference levels of disease-related gene expression for a reference population, also referred to as control population. When relative tissue contributions are considered individually, quantification of each of these liver disease stage-related transcripts can be used as a measure of a reference apoptotic rate, cell turnover rate, senescence rate, nucleic acid release rate, or secretion rate of that particular tissue for that particular population. For example, blood from one or more healthy, normal individuals can be analyzed to determine the relative RNA contribution of tissues to the cell-free RNA transcriptome for healthy, normal individuals. Each relative RNA contribution of tissue that makes up the normal RNA transcriptome can be a reference level for that tissue.

Some methods disclosed herein comprise deducing relative contributions of different tissue types. A quantified panel of tissue-specific transcripts can be considered as a summation of the contributions from the various tissues. Relative contributions of different tissue types may be obtained by inserting observed transcript levels in a sample tissue and a reference tissue into the following equation to determine πᵢ for each tissue, which will correspond to the fractional contribution the sample tissue(s) to the cell-free transcriptome.

$${Y}_{i} = {\sum }_{j} {π}_{i} {X}_{ij} + ɛ$$

Where Y is the observed transcript quantity in a sample for gene i, X is the known transcript quantity for gene i in a reference tissue j and ε the normally distributed error.

Additional physical constraints include:

1. Summation of all fraction contributing to the observed quantification is 1, given by the condition: Σπi=1.

2. All the contribution from each tissue type has to be greater than or equal zero. There is no physical meaning to having a negative contribution. This is given by πi≥0, since Σ is defined as the fractional contribution of each tissue types.

Consequently, to obtain the optimal fractional contribution of each tissue type, the least-square error is minimized. The above equations are then solved using quadratic programming in R to obtain the optimal relative contributions of the tissue types towards the reference cell free RNA transcripts. In the workflow, the quantity of RNA transcripts are given relative to the housekeeping genes in terms of Ct values obtained from qPCR. Therefore, the Ct value can be considered as a proxy of the measured transcript quantity. An increase in Ct value of one is similar to a two-fold change in transcript quantity, i.e., 2 raised to the power of 1. The process begins with normalizing all of the data in CT relative to the “housekeeping” gene and is followed by quadratic programming.

### Kits and Systems

As discussed in the foregoing and following description, systems and kits are provided herein that can non-invasively detect whether a liver in a subject is under duress as well as determine which liver disease stage or condition is affecting the liver under duress. Disclosed herein are kits for use in detecting a liver disease stage or condition in a subject, the kit can comprise at least one reagent for detecting at least one marker and at least one reagent for detecting at least one liver disease stage-related polynucleotide. Additionally, or alternatively, the kits disclosed herein may be used to determine the location (e.g., tissue) and/or progression of a liver disease stage or condition in the subject. Additionally, or alternatively, the kits disclosed herein may be used to determine if a therapy administered to the subject has affected the progression of the liver disease stage or condition. Additionally, or alternatively, the kits disclosed herein may be used to determine if a therapy administered to the subject has resulted in any unintended toxicity or side effects.

Provided herein are kits that can comprise at least one reagent disclosed herein. The at least one reagent for detecting liver disease stage-related polynucleotides may comprise at least one reagent for detecting a cell-free polynucleotide. The at least one reagent for detecting at least one marker may comprise at least one reagent for a detecting cell-free polynucleotide. The at least one cell free polynucleotide may comprise cell-free DNA or cell-free RNA. The cell-free DNA may have a disease-related methylation pattern. The cell-free polynucleotide may be a liver disease stage-related gene transcript. The at least one reagent for detecting at least one marker and/or the at least one reagent for detecting the liver disease stage-related polynucleotide may comprise a polynucleotide probe. The polynucleotide probe may bind to the cell-free polynucleotide. The polynucleotide probe may bind to the cell-free polynucleotide in a sequence-dependent manner. The polynucleotide probe may bind to a cell-free polynucleotide corresponding to a wildtype version of a gene but not a mutant version of the gene. Alternatively, the polynucleotide probe may bind to a cell-free polynucleotide corresponding to a mutant version of a gene but not a wildtype version of the gene. The polynucleotide probe may be attached or coupled to a signaling moiety. By way of non-limiting example, the signaling moiety may be selected from a hapten, a fluorescent molecule, and a radioactive isotope. The kit may be specific for one liver disease or condition. The kit may comprise as few as 1, 2, 3, 4, or 5 polynucleotide probes in order to detect a liver disease or condition in a subject. The kit may be specific for multiple liver diseases or conditions. The kit may comprise from about 5 to about 10, about 10 to about 20, about 10 to about 100, about 10 to about 1000, about 100 to about 1000, or about 100 to about 10,000 polynucleotide probes.

Provided herein are kits that comprise at least one reagent disclosed herein. The at least one reagent for detecting at least one marker and/or the at least one reagent for detecting the liver disease stage-related polynucleotide may comprise a primer. The primer may be a reverse transcriptase primer. The primer may be a PCR primer. The primer may amplify the at least one marker, at least one disease-related polynucleotide, or portions thereof. The primer may amplify the cell-free polynucleotide in a sequence-dependent manner. The primer may amplify a cell-free polynucleotide or portion thereof corresponding to a wildtype version of a gene, but not a mutant version of the gene. Alternatively, the primer may amplify a cell-free polynucleotide or portion thereof corresponding to a mutant version of a gene, but not a wildtype version of the gene. The kit may further comprise an amplification reporter that provides a user of the kit with the quantity of the at least one marker and/or the at least one reagent for detecting the liver disease stage-related polynucleotides. Typically, the quantity is a relative quantity based on a reference sample. The amplification signaling reagent may be selected from intercalating fluorochromes or dyes. The amplification signaling reagent may be SYBR Green.

Provided herein are kits that can comprise at least one reagent disclosed herein. The at least one reagent for detecting at least one marker and/or the at least one reagent for detecting the liver disease stage-related polynucleotide may comprise a peptide that binds to the at least one marker or liver disease stage-related polynucleotide. The peptide may be part of an antibody, or a polynucleotide binding protein (e.g., transcription factor, histone, etc.). The at least one reagent for detecting at least one marker and/or the at least one reagent for detecting the liver disease stage-related polynucleotide may comprise a signaling moiety that emits a signal, wherein the signal being emitted or lost may be indicative of a presence or a quantity of a marker or a liver disease stage-related polynucleotide. Examples of signaling moieties include, but are not limited to, dyes, fluorophores, enzymes, and radioactive particles. The at least one reagent may further comprise a signaling moiety detector for detecting the signal or absence thereof.

Disclosed herein are kits for use in detecting whether or not a liver is affected by a stage of liver disease, wherein the kits can comprise at least one probe or primer for a marker of the condition. In some instances, the kits may comprise at least one probe and at least one primer. In some instances, the marker may be a polynucleotide and the primer or probe may be a polynucleotide that hybridizes to a target of interest. In some instances, the marker can be a peptide or protein and the probe can be an antibody or antibody fragment capable of binding the peptide or protein. In some instances, the probe can be a small molecule that binds to the marker. In some instances, the probe can be conjugated to a tag that can be used to retrieve the marker, quantify the marker, or detect the marker. The at least one liver disease stage may also include at least one of: inflammation, apoptosis, necrosis, fibrosis, infection, or autoimmune disease.

Disclosed herein are kits for use in detecting a liver disease stage or condition in a subject, the kit comprising at least one reagent for detecting at least one marker and at least one reagent for detecting at least one liver disease stage-related polynucleotide. The kit may further comprise a solid support, wherein the polynucleotide probe, the primer and/or the peptide is attached to a solid support. The solid support may be selected from a bead, a chip, a gel, a particle, a well, a column, a tube, a probe, a slide, a membrane, and a matrix.

Disclosed herein are kits for use in detecting a liver disease stage or condition in a subject, the kit can comprise at least one reagent for detecting at least one marker and at least one reagent for detecting at least one liver disease stage-related polynucleotide. The two or more components of the kits disclosed herein may be separate. The two or more components of the kits disclosed herein may be integrated. The two or more components of the kits disclosed herein may be integrated into a device. The device may allow for a user to simply add at least one sample from the subject to the device and receive a result indicating whether or not the subject has the liver disease stage or condition and/or which tissue(s) of the subject is affected by the liver disease stage or condition. In some cases, the user may add at least one reagent to the device. In other cases, the user may not add (e.g., may not have to add) any reagents to the device.

Disclosed herein are kits for use in detecting a liver disease stage or condition in a subject, the kit can comprise at least one reagent for detecting at least one marker and at least one reagent for detecting at least one liver disease stage-related polynucleotide. The at least one liver disease stage-related polynucleotide or marker may comprise a cell free polynucleotide. The at least one marker may comprise RNA. The at least one liver disease stage-related polynucleotide may comprise at least one tissue specific RNA, wherein a tissue specific RNA is an RNA expressed only in a specific tissue or at a level in a specific tissue that is substantially higher than the level at which it is expressed in other tissues. For example, a tissue-specific gene may be a gene for which expression in a particular tissue or group of tissues is at least 2-fold, 5-fold, 10-fold, or 25-fold greater than any other tissue or group of tissues (e.g., any individually, or all other tissues or group of tissues combined). The at least one disease-related polynucleotide or marker may comprise at least one disease-related methylated DNA, wherein the disease-related methylated DNA may comprise a disease-related methylation pattern. Alternatively, or additionally, the disease-related methylated DNA may comprise DNA with a methylation pattern that occurs in only one tissue or at a level in a tissue that is substantially higher than the level at which it occurs in other tissues. The tissue may be determined to be damaged by the condition if (a) the level of at least one of the marker is above the reference level of the at least one marker and (b) the level of at least one of the disease-related polynucleotide is above the reference level of the at least one disease-related polynucleotide. The at least one disease-related polynucleotide may comprise two or more polynucleotides each of which is specific for a different tissue (e.g., 2, 3, 4, 5, 10, 15, 25, or more different tissues). The tissue may be liver tissue. The marker and/or liver disease stage-related polynucleotide may correspond to a gene. In general, a marker or liver disease stage-related polynucleotide “corresponds to a gene,” as used herein, if it is a DNA molecule comprising the gene (or an identifiable portion thereof) or is an expression product of the gene (e.g., an RNA transcript or a protein product).

Further disclosed herein are systems for carrying out methods of the present disclosure. In general, a system may comprise various units capable of performing the steps of methods disclosed herein, for example a sample processing unit, an amplification unit, a sequencing unit, a detection unit, a quantifying unit, a comparing unit, and/or a reporting unit. In some embodiments, the system may comprise: a memory unit configured to store results of (i) an assay for detecting at least one marker of at least one condition in a first sample of a subject and (ii) an assay for detecting at least one disease-related RNA in a second sample of a subject, wherein the at least one liver disease stage-related RNA is a cell-free RNA specific to a tissue; at least one processors programmed to: (i) quantify a level of the at least one marker; (ii) quantify a level of the at least one liver disease stage-related polynucleotide; (iii) compare the level of the at least one marker to a corresponding reference level of the marker; (iv) compare the level of the at least one liver disease stage-related polynucleotide to a corresponding reference level of the liver disease stage-related polynucleotide; and (v) determine presence of or relative change in damage of the liver by the at least one condition based on the comparing; and an output unit that delivers a report to a recipient, wherein the report provides results of step (b). The system may provide a recommendation for medical action based on the results of step (b). The medical action may comprise a treatment. The first sample and the second sample may be the same. The first sample and the second sample may be different. The first sample and the second sample may be different in that they were obtained at different times. The first sample and the second sample may be different in that they are different fluids. The first and/or second sample may be a fluid selected from the group consisting of: whole blood, blood plasma, blood serum, a blood fraction, saliva, sputum, urine, semen, a transvaginal fluid, a cerebrospinal fluid, sweat, or a breast fluid. The first and/or second sample may be blood plasma or serum.

The systems disclosed herein may be used with any one of the kits or devices disclosed herein. The systems may be integrated with any one of the kits or devices disclosed herein. The devices disclosed herein may comprise any one of the systems disclosed herein. In some embodiments, the system may comprise a computer system. A computer for use in the system may comprise at least one processor. Processors may be associated with at least one controller, calculation unit, and/or other unit of a computer system, or implanted in firmware as desired. If implemented in software, the routines may be stored in any computer readable memory such as in RAM, ROM, flashes memory, a magnetic disk, a laser disk, or other suitable storage medium. Likewise, this software may be delivered to a computing device via any known delivery method including, for example, over a communication channel such as a telephone line, the Internet, a wireless connection, etc., or via a transportable medium, such as a computer readable disk, flash drive, etc. The various steps may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc. A client-server, relational database architecture can be used in embodiments of the system. A client-server architecture is a network architecture in which each computer or process on the network is either a client or a server. Server computers are typically powerful computers dedicated to managing disk drives (file servers), printers (print servers), or network traffic (network servers). Client computers include PCs (personal computers) or workstations on which users run applications, as well as example output devices as disclosed herein. Client computers rely on server computers for resources, such as files, devices, and even processing power. In some embodiments, the server computer handles all of the database functionality. The client computer can have software that handles all the front-end data management and can also receive data input from users.

Systems disclosed herein may be configured to receive a user request to perform a detection reaction on a sample. The user request may be direct or indirect. Examples of direct request include those transmitted by way of an input device, such as a keyboard, mouse, or touch screen). Examples of indirect requests include transmission via a communication medium, such as over the Internet (either wired or wireless).

Systems disclosed herein may further comprise a report generator that sends a report to a recipient, wherein the report contains results of a method described herein. A report may be generated in real-time, such as during a sequencing read or while sequencing data is being analyzed, with periodic updates as the process progresses. In addition, or alternatively, a report may be generated at the conclusion of the analysis. In some embodiments, the report is generated in response to instructions from a user. In addition to the results of detection or comparison, a report may also contain an analysis, conclusion or recommendation based on such results. For example, markers associated with a liver disease stage or condition are detected and levels of a liver disease stage-related polynucleotide are above a normal range, the report may include information concerning this association, such as a likelihood that subject has the liver disease stage or condition, which tissues are or are not affected, and/or a suggestion based on this information (e.g., additional tests, monitoring, or remedial measures). The report can take any of a variety of forms. It is envisioned that data relating to the present disclosure can be transmitted over such networks or connections (or any other suitable means for transmitting information, including, but not limited to, mailing a physical report, such as a print-out) for reception and/or for review by a receiver. The receiver can be, but is not limited to, an individual or electronic system (e.g., at least one computers and/or at least one server).

The disclosure provides a computer-readable medium comprising code that, upon execution by at least one processor, implements a method of the present disclosure. A machine readable medium comprising computer-executable code may take many forms, including, but not limited to, a tangible storage medium, a carrier wave medium, or physical transmission medium. Non-volatile storage media include, for example, optical or magnetic disks, such as any of the storage devices in any computers or the like, such as may be used to implement the databases, etc. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include, for example: a floppy disk, a flexible disk, hard disk, magnetic tape, any other magnetic medium, a CD-ROM, DVD or DVD-ROM, any other optical medium, punch cards, paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying at least one sequence of at least one instruction to a processor for execution.

As discussed in the foregoing and following description, methods, systems and kits are provided herein that can non-invasively detect a liver under duress as well as determine which liver disease stage or condition is affecting the liver under duress. Provided herein are kits, devices, systems and methods employing liver disease-related gene expression, liver disease-related nucleic acids (e.g., RNAs) and liver disease-related nucleic acid modifications (e.g., methylation patterns). The terms, “liver disease-related nucleic acid” and “liver disease-related polynucleotide,” are interchangeable as used herein. The term “liver disease-related,” as used herein, is generally used to characterize a nucleic acid that is expressed during the normal functioning of a subject's liver. Alternatively, the term “liver disease-related,” as used herein, is generally used to characterize a nucleic acid that is predominantly expressed in a liver of the subject. For the purposes of this application, predominantly expressed may mean that the liver disease-related nucleic acid is expressed at an RNA level that is at least 50% greater in liver tissue than the RNA level of the liver disease-related nucleic acid in a liver tissue of a subject without liver disease or a liver under distress. However, in some cases, a liver disease-related nucleic acid expressed at an RNA level that is at least 30% greater in liver tissue than that of a liver tissue of a subject without liver disease or a liver under distress may be sufficient for the methods disclosed herein. In other cases, a disease-related nucleic acid expressed at an RNA level that is at least 80% greater in an individual with a stage of liver disease than an individual without a stage of a liver disease by the methods disclosed herein.

Provided herein are kits, systems and methods for detecting or quantifying a biological molecule in a sample from a subject, including by way of non-limiting example, polynucleotides, peptides/proteins, lipids, and sterols. Biological molecules disclosed herein may be liver disease stage-related. The term “liver disease stage-related,” as used herein, generally refers to a biological molecule, or modification thereof, that is expressed at a higher level in liver tissue at particular stage of liver disease than that of a liver tissue of a subject without liver disease or a liver in a different stage of liver disease.

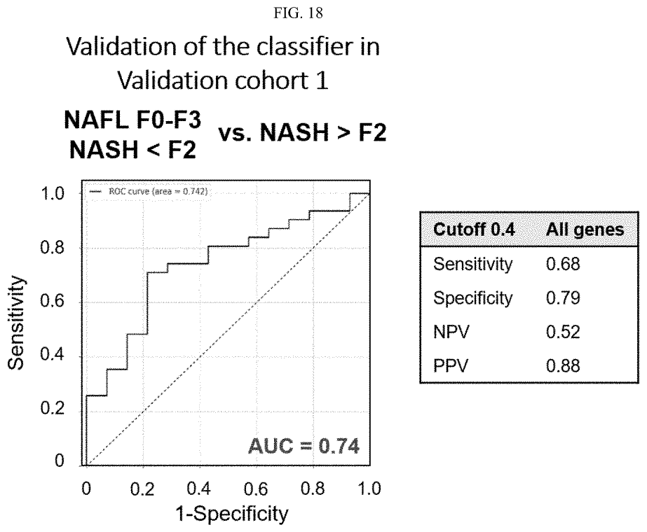

Provided herein are kits, systems and methods for detecting or quantifying a liver disease stage-related polynucleotide in a sample. At least one database of genetic information can be used to identify a liver disease stage-related polynucleotide or a panel of liver disease stage-related polynucleotides. Accordingly, aspects of the disclosure provide systems and methods for the use and development of a database. Methods of the disclosure may utilize databases containing existing data generated across tissue types to identify the disease-related genes. Such databases may be utilized for identification of liver disease stage-related genes. The database may be a web-based gene expression profile. Non-limiting examples of web-based gene expression repositories are publicly available, e.g., The Human Protein Atlas at www.proteinatlas.org, BioGPS at biogps.org and The European Bioinformatics Institute Expression Atlas at www.ebi.ac.uk/gxa/, Gene Expression Omnnibus (GEO) at ncbi.nlm.nih.gov/geo/, the content of all of which are incorporated herein by reference. Such databases are also publicly available as published articles in printed and on-line journals. Databases may also be referred to as atlases, e.g., the Human 133A/GNF1H Gene Atlas (see Su et al., Proc Natl Acad Sci USA, 2004, vol. 101, pp. 6062-7 for original publication) and RNA-Seq Atlas (see Krupp et al., Bioinformatics, 2012, vol. 15, pp. 1184-5 for original publication), which are both incorporated herein by reference. These databases and websites incorporate data from many independent studies and often corroborate tissue specific gene expression patterns amongst a species. Such cross-validation can provide useful liver disease stage-related polynucleotides for methods, systems and kits disclosed herein. In some instances, a liver disease stage-related polynucleotide disclosed herein can be identified as having tissue-specific expression by at least two published datasets. In some instances, a liver disease stage-related polynucleotide disclosed herein may be identified as having tissue specific expression by at least three published datasets. In some instances, a liver disease stage-related polynucleotide disclosed herein may be identified as having tissue specific expression by at least four published datasets. In some instances, a liver disease stage-related polynucleotide disclosed herein can be identified as having tissue specific expression by at least five published datasets. In order to identify liver disease stage-related transcripts from at least one database, certain embodiments employ a template-matching algorithm to the databases. Template matching algorithms used to filter data are known, see, e.g., Pavlidis P, Noble W S (2001) Analysis of strain and regional variation in gene expression in mouse brain. Genome Biol 2:research0042.1-0042.15. Examples of tissue-specific genes include those appearing in FIG. 18 of US20130252835, which is incorporated herein by reference.

Provided herein are kits, systems and methods for detecting or quantifying a liver disease-stage related polynucleotide in a sample. The liver disease stage-related nucleic acid may refer to a nucleic acid that is expressed in a liver of each subject in a population of subjects. The liver disease stage-related nucleic acid may refer to a nucleic acid that is predominantly expressed in a liver of each subject in a population of subjects. The population of subjects may be healthy. The population of subjects may have a common liver disease stage or condition. The population of subjects may comprise at least two subjects. The population of subjects may comprise at least five subjects. The population of subjects may comprise at least ten subjects. The population of subjects may comprise at least twenty subjects. The population of subjects may have a common ethnicity, a common genetic background, a common BMI, a common metabolic disorder, a common gender, a common age, or a combination thereof. The liver disease stage-related nucleic acid may refer to a nucleic acid that is expressed in a liver, or predominantly expressed in a liver, as shown by, for example, a published study or database. The published study may have employed microarray technology or RNA-seq profiling to measure tissue specific nucleic acid levels. In some instances, damage of the liver is caused by a liver disease stage or condition resulting in apoptosis of cells in the liver, releasing cell-free liver disease stage-related nucleic acids into a circulating fluid of the subject. The liver disease stage-related nucleic acid may be a nucleic acid that is expressed highly enough in the liver that it can be detected in a circulating biological fluid (e.g., blood, plasma, etc.) when damage to the liver occurs. The liver disease stage-related nucleic acid may be a nucleic acid that is expressed highly enough in the liver that it can be detected in a circulating biological fluid (e.g., blood, plasma, etc.) when damage to at least about 10%, at least about 20%, at least about 30%, at least about 40% or at least about 50% of the liver occurs.

Disclosed herein are methods, kits and systems for detecting, quantifying, and/or analyzing liver disease stage-related polynucleotides. In general, the liver disease stage-related polynucleotides can be cell-free polynucleotides, released into a biological fluid (e.g., blood, cerebrospinal fluid, lymphatic fluid, urine, etc.), upon damage, inflammation, or injury to a liver. As used herein, damage or injury to the liver may be due to a liver disease stage or condition that results in disruption of a cell membrane or a loss of cell membrane integrity of at least one cell within or on the surface of the liver. Damage or injury to the liver may be due to a liver disease stage or condition that results in inflammation, hepatic fibrosis, or regeneration associated with the stage of liver disease. Disruption of the cell membrane or loss of cell membrane integrity may result in a release of polynucleotides within the cell. Disruption of the cell membrane may be due, for instance, to necrosis, autolysis, or apoptosis. Regeneration associated with the stage of liver disease may result in a release of polynucleotides. Non-limiting examples of liver disease stage-related polynucleotides include liver disease stage-related RNA and DNA comprising a disease-related methylation pattern. Disease-related RNAs may include, but are not limited to, a messenger RNA (mRNA), a microRNA (miRNA), a pre-miRNA, a pre-miRNA, a pre-mRNA, a circular RNA (circRNA), a long non-coding RNA (lncRNA), and an exosomal RNA. Examples of genes having liver disease stage-related expression are provided herein.

Provided herein are kits, systems and methods for detecting or quantifying a biological molecule in a sample from a subject for Research and Development applications. Biological molecules disclosed herein may be liver disease-related. The term “liver disease-related,” as used herein, generally refers to a biological molecule, or modification thereof, that is expressed at a higher level in a subject with a liver disease than a subject without the liver disease or stage of liver disease. The liver disease-related biological molecule can be cell-free mRNA as disclosed herein. The disease can be liver disease wherein the subject is classified with one of the following stages of liver disease: no observable fibrosis (F0), portal fibrosis without septa (F1), portal fibrosis with few septa (F2), bridging septa between central and portal veins (F3), and cirrhosis (F4)

The detection or quantification of disease-related biological molecules (e.g., liver disease-related biological markers) can be used for pre-clinical therapeutic target discovery. The detection or quantification of disease-related biological molecules can be used for pre-clinical measurement of target engagement. The detection or quantification of disease-related biological molecules can be used to track, detect, and measure targets of interest for therapy/drug discovery and development.

The detection or quantification of disease-related cell-free mRNA (e.g., liver disease-related cell-free mRNA) can be used to determine gene signatures and biomarker discovery for patient stratification in pre-clinical and clinical studies.

The detection or quantification of disease-related cell-free mRNA (e.g., liver disease-related cell-free mRNA) can be used to optimize late-stage lead molecule optimization for further clinical development. The detection or quantification of disease-related cell-free mRNA can be used to measure pharmacodynamics for lead optimization and clinical development during therapy/drug discovery and development. The detection or quantification of disease-related cell-free mRNA can be used to create a profile of gene expression that characterizes the pharmacodynamic effect associated with the engagement of a specific target for therapy/drug discovery and development. The detection or quantification of disease-related cell-free mRNA can be used to detect changes in pharmacodynamic target engagement for therapy/drug discovery and development.

The detection or quantification of disease related cell-free mRNA (e.g., liver disease-related cell-free mRNA) can be used to measure target molecule engagement in the early clinical development of pharmaceutical candidates to treat the disease. The detection or quantification of disease related cell-free mRNA can be used in methods to select candidates for IND filings. The detection or quantification of disease related cell-free mRNA (e.g., liver disease-related cell-free mRNA) can be used to measure target molecule engagement at time points periodically over a set period of time. The time points can be equal to or less than every 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, or any other suitable period of time. The time points can be equal or greater than every 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, or any other suitable period of time. The set period of time can be less than or equal to 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 2 years, 3 years, 4 years, 5 years, or 10 years. The set period of time can be greater than or equal to 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 2 years, 3 years, 4 years, 5 years, or 10 years.

The detection or quantification of disease related cell-free mRNA (e.g., liver disease-related cell-free mRNA) can be used to develop endpoints to evaluate the relative therapeutic efficacy of therapeutic agents administered to a subject.

The development of cell-free mRNA disease signatures (e.g., cell-free mRNA liver disease signatures) can be used to evaluate the relative toxicity of candidate therapeutic agents or a subject's response to therapeutic agents. For example, a subject receiving a first prescription for a first disease may then be able to be tracked closely for toxic interactions between a pharmaceutical within the first prescription administered and a candidate therapeutic by monitoring the liver-disease related cell-free mRNA gene panels as disclosed herein.

A liver disease-related biological molecule can be a biological molecule, or modification thereof, that is expressed at a higher level in liver tissue than in any other tissue in the subject. A liver disease-related biological molecule can be a biological molecule, or modification thereof, that is expressed at a higher level in hepatocytes of the liver. In some instances, it is expressed at least 10% higher in liver tissue than in any other tissue in the subject. In some instances, it is expressed at least 20% higher in liver tissue than in any other tissue in the subject. In some instances, it is expressed at least 30% higher in liver tissue than in any other tissue in the subject. In some instances, it is expressed at least 40% higher in liver tissue than in any other tissue in the subject. In some instances, it is expressed at least 50% higher in liver tissue than in any other tissue in the subject. Thus, the liver disease-related biological molecule may be considered predominantly present or predominantly expressed in liver tissue. Disease-related biological molecules disclosed herein may be disease-related polynucleotides. Disease-related polynucleotides are nucleic acids that are expressed or modified in a disease-related manner. For example, there may be only a single tissue or organ, or small set of tissues or organs that predominantly accounts for the expression of a particular gene (e.g., 60-80%, 90%, 95% or more of a gene's total expression in the subject).

In some instances, methods disclosed herein can comprise comparing the level of a single disease-related polynucleotide to a corresponding reference level of the disease-related polynucleotide can be sufficient to determine whether a tissue has been damaged by a liver disease or condition. In other instances, the level of multiple disease-related polynucleotides may be compared to corresponding reference levels of the disease-related polynucleotides to determine whether a tissue has been damaged by a liver disease or condition. The methods disclosed herein may comprise comparing the level of as few as 1, 2, 3, 4, 5, 6, 7, 8, 9, or 10 disease-related polynucleotides to corresponding reference levels to determine whether a tissue that has been damaged by a liver disease or condition. There may be an advantage to comparing as few as 1, 2, or 3 disease-related polynucleotides to corresponding reference levels.

In some instances, methods disclosed herein of comparing the level of a disease-related polynucleotide to a corresponding reference level of the disease-related polynucleotide may result in determining that the level of the disease-related polynucleotide is greater than the corresponding reference level. In some cases, the corresponding reference level may be the level of the disease-related polynucleotide in a healthy individual and the level of the disease-related polynucleotide being greater than the corresponding reference level may be indicative of damage or injury to a specific tissue, organ, or cell in the subject. The level of the disease-related polynucleotide may be at least about 5%, at least about 10%, at least about 20%, at least about 30%, at least about 40%, at least about 50%, at least about 60%, at least about 70%, at least about 80%, at least about 90%, at least about 100%, at least about 150%, or at least about 200% greater than the corresponding reference level.

In some instances, methods disclosed herein of comparing the level of a disease-related polynucleotide to a corresponding reference level of the disease-related polynucleotide may result in determining that the level of the disease-related polynucleotide is lower than the corresponding reference level. In some cases, the corresponding reference level can be the level of the disease-related polynucleotide in an individual or population having the liver disease or condition, and the level of the disease-related polynucleotide being lower than the corresponding reference level can be indicative of the absence of or a minimal amount of damage or injury to a specific tissue, organ, or cell in the subject. The level of the disease-related polynucleotide may be at least about 5%, at least about 10%, at least about 20%, at least about 30%, at least about 40%, at least about 50%, at least about 60%, at least about 70%, at least about 80%, at least about 90%, or at least about 95% lower than the corresponding reference level.

One way to define any known variants and derivatives or those that might arise, of the disclosed nucleic acids and polypeptides herein, is through defining the variants and derivatives in terms of homology to specific known sequences. In general, variants of nucleic acids and polypeptides herein disclosed typically have at least, about 80%, 81%, 82%, 83%, 84%, 85%, 86%, 87%, 89%, 90%, 91%, 92%, 93%, 94%, 95%, 96%, 97%, 98%, 98.5%, 99%, 99.5% or 99.9% homology to the stated sequence or the native sequence. Those of skill in the art readily understand how to determine the homology of two polypeptides or nucleic acids. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.; the BLAST algorithm of Tatusova and Madden FEMS Microbiol. Lett. 174: 247-250 (1999) available from the National Center for Biotechnology Information (http://www.ncbi.nlm.nih.gov/blast/b12seq/b12.html), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989, which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

Liver disease stage-related polynucleotides disclosed herein may be described as “corresponding to a gene.” In some instances, the phrase “corresponding to a gene,” as used herein, generally means the disease-related polynucleotide is transcribed from a gene. Thus, in some instances, disease-related polynucleotides are disease-related RNA transcripts. Disease-related RNA transcripts include, but are not limited to, full-length transcripts, transcript fragments, transcript splice variants, enzymatically or chemically cleaved transcripts, transcripts from two or more fused genes, and transcripts from mutated genes. Fragments and cleaved transcripts must retain enough of the full-length polynucleotide to be recognizable as correspond to the gene. In some instances, 5% of the full-length polynucleotide is enough of the fill-length polynucleotide. In some instances, 10% of the full-length polynucleotide is enough of the full-length polynucleotide. In some instances, 15% of the full-length polynucleotide is enough of the full-length polynucleotide. In some instances, 20% of the full-length polynucleotide is enough of the full-length polynucleotide. In some instances, 25% of the full-length polynucleotide is enough of the full-length polynucleotide. In some instances, 30% of the full-length polynucleotide is enough of the full-length polynucleotide. In some instances, 40% of the full-length polynucleotide is enough of the full-length polynucleotide. In some instances, 50% of the full-length polynucleotide is enough of the full-length polynucleotide. In some instances, the phrase “corresponding to a gene” means the disease-related polynucleotide is a modified form of the gene (e.g., disease-related DNA modification pattern).

### Markers of a Liver Disease Stage or Condition

As discussed in the foregoing and following description, methods, systems and kits are provided herein that can non-invasively detect a tissue or organ under duress as well as determine which liver disease stage or condition is affecting the tissue or organ under duress. Disclosed herein are methods, kits and systems for detecting, quantifying, and/or analyzing at least one marker of a liver disease or condition. Similar to the liver disease stage-related polynucleotides disclosed herein, a marker may be a cell-free polynucleotide, released into a biological fluid (e.g., blood, cerebrospinal fluid, lymphatic fluid, urine, etc.), upon damage, injury, or regeneration of a liver. In some cases, the at least one marker of the liver disease stage or condition may comprise a liver disease stage-related polynucleotide disclosed herein. Damage or injury to the liver may be due to a liver disease stage or condition that results in cytolysis within or on the surface of the tissue or organ. Regeneration of the liver may be due to a liver disease stage or condition that results in cytolysis within or on the surface of the tissue or organ.

Markers disclosed herein, by way of non-limiting example, may be selected from a peptide, a protein, an aptamer, an antibody, a cell fragment, a sterol (e.g., cholesterol), a hormone, a lipid, a phospholipid, a fatty acid, a sugar moiety, a vitamin, a metabolite, and an extracellular matrix component, complexes thereof, and chemical modifications thereof. Chemical modifications may include, but are not limited to, phosphorylation, myristoylation, palmitoylation, acetylation, methylation, sumoylation, glycosylation, and ubiquitination. The methods disclosed herein may comprise an assay to detect these markers. A variety of suitable assays are available, selection of which may depend on the type of marker to be detected. By way of non-limiting example, these assays include ELISA, western blot, gel electrophoresis, and reporter assays. Any suitable number of markers for any or more liver diseases or conditions may be assayed in parallel or in a single reaction. For example, an assay may comprise detecting at least 5, 10, 25, 50, 75, 100, 250, 500, 1000, or more markers, for the assessment of at least 1, 2, 3, 4, 5, 10, 15, 25, or more liver diseases or conditions. Any convenient assay format for such multiplexed reactions may be employed, examples of which are provided herein, including, but not limited to, microarray analysis and high-throughput sequencing methodologies.

Disclosed herein are methods, kits and systems for detecting, quantifying, and/or analyzing at least one marker of a liver disease stage or condition, wherein the marker is a cell-free polynucleotide. Non-limiting examples of cell-free polynucleotides as markers include RNA and DNA (including DNA comprising a disease-related methylation pattern). Examples of RNA useful as a marker for a liver disease or condition include, but are not limited to, messenger RNA (mRNA), microRNA (miRNA), pre-miRNA, pri-miRNA, pre-mRNA, eukaryotic RNA, prokaryotic RNA, viral RNA, bacterial RNA, parasitic RNA, fungal RNA, viroid RNA, virusoid RNA, circular RNA (circRNA), ribosomal RNA (rRNA), transfer RNA (tRNA), pre-tRNA, long non-coding RNA (lncRNA), small nuclear RNA (snRNA), and exosomal RNA. DNA may include single-stranded DNA, double-stranded DNA, DNA-protein complexes, mitochondrial DNA, bacterial DNA, and DNA with specific chemical modification patterns (e.g., methylated DNA). Bacterial DNA/RNA may include those of gut organisms and may be markers of a dietary sensitivity, gut condition, or metabolic condition.

The presence, or relative or absolute quantity of the at least one marker in a subject's sample may be indicative of the presence, stage, or progression of a liver disease stage or condition, a response to a therapy administered to the subject to treat the liver disease stage or condition, or indicative of how a subject might respond to a particular treatment. In some cases, a lower level of the at least one marker in the sample relative to a reference level may be indicative of the presence, stage, or progression of a liver disease or condition, or a response to a therapy administered to the subject to treat the liver disease stage or condition. In some cases, a higher level of the at least one marker in the sample relative to a reference level may be indicative of the presence, stage, or progression of a liver disease stage or condition, or a response to a therapy administered to the subject to treat the liver disease stage or condition. A mutation or specific sequence of the at least one marker may be indicative of the presence or progression of a liver disease stage or condition, or a response to a therapy administered to the subject to treat the liver disease stage or condition. The quantity of the at least one marker with a specific mutation or sequence may be indicative of the presence or progression of a liver disease stage or condition, or a response to a therapy administered to the subject to treat the liver disease stage or condition.

Markers disclosed herein may be described as “corresponding to a gene.” In some instances, the phrase “corresponding to a gene,” as used herein, generally means the marker is transcribed from a gene. Thus, in some instances, a marker is a RNA transcript. RNA transcripts include, but are not limited to, full-length transcripts, transcript fragments, transcript splice variants, enzymatically or chemically cleaved transcripts, transcripts from two or more fused genes, and transcripts from mutated genes. Fragments and cleaved transcripts must retain enough of the full-length polynucleotide to be recognizable as corresponding to the gene. In some instances, 5% of the full-length polynucleotide is enough of the full-length polynucleotide. In some instances, 10% of the full-length polynucleotide is enough of the full-length polynucleotide. In some instances, 15% of the full-length polynucleotide is enough of the full-length polynucleotide. In some instances, 20% of the full-length polynucleotide is enough of the full-length polynucleotide. In some instances, 25% of the full-length polynucleotide is enough of the full-length polynucleotide. In some instances, 30% of the full-length polynucleotide is enough of the full-length polynucleotide. In some instances, 40% of the full-length polynucleotide is enough of the full-length polynucleotide. In some instances, 50% of the full-length polynucleotide is enough of the full-length polynucleotide.

In some instances, the phrase “corresponding to a gene,” as used herein, generally means the disease-related polynucleotide is a modified form of the gene (e.g., disease-related DNA modification pattern). In some instances, the phrase “corresponding to a gene,” as used herein, generally means the marker is a protein encoded by a gene. The protein may be a full-length protein, a cleaved protein, a protein fragment, a pro-form of a protein (e.g., before naturally occurring enzymatic cleavage), an insoluble version of the protein, a soluble protein, a secreted protein, a protein that is released from a cell upon cell death, or a protein that is released from a tissue upon tissue damage. Fragments and cleaved proteins must retain enough of the full-length protein to be recognizable as corresponding to the gene. In some instances, 5% of the full-length protein is enough of the full-length protein. In some instances, 10% of the full-length protein is enough of the full-length protein. In some instances, 15% of the full-length protein is enough of the full-length protein. In some instances, 20% of the full-length protein is enough of the full-length protein. In some instances, 25% of the full-length protein is enough of the full-length protein. In some instances, 30% of the full-length protein is enough of the full-length protein. In some instances, 40% of the full-length protein is enough of the full-length protein. In some instances, 50% of the full-length protein is enough of the full-length protein.

### Liver Disease Stages and Conditions

As discussed in the foregoing and following description, methods, systems and kits are provided herein that can non-invasively detect a liver under duress as well as determine which liver disease stage or condition is affecting the liver under duress. Methods, kits and systems disclosed herein may provide for detecting, quantifying, and/or analyzing at least one marker of a liver disease stage or condition. By way of non-limiting example, repeated damage and regeneration of a liver may result in permanent damage or injury to the liver. Damage to a liver may result in cell death, inflammation, hepatic stellate cell activation, cell lysis, or cell membrane disruption, resulting in the release of nucleic acids from respective cells and the presence of cell-free, disease-related polynucleotides in biological fluids (e.g., blood, plasma, serum, cerebrospinal fluid, etc.) of the subject. Any of a variety of liver disease stages or conditions may be assessed using methods of the disclosure, either alone or in combination. Non-limiting examples of liver disease stages or conditions include NALFD, no observable fibrosis (F0), portal fibrosis without septa (F1), portal fibrosis with few septa (F2), bridging septa between central and portal veins (F3), cirrhosis (F4), and NASH. Conditions can include non-disease conditions of a subject. For example, conditions of a subject can include likelihood of response to a mode of treatment (e.g., a pharmaceutical composition) determined prior to administration, and degree of positive or negative response to such treatment after administration.

### EXAMPLES

The application may be better understood by reference to the following non-limiting examples, which are provided as exemplary embodiments of the application. The following examples are presented in order to more fully illustrate embodiments and should in no way be construed, however, as limiting the broad scope of the application.

### Example 1: Analysis of Whole Transcriptome Cf-mRNA in Clinically Characterized NAFLD Patient Cohorts

Whole transcriptome circulating-free messenger RNA (cf-mRNA) expression analysis was performed in clinically characterized NAFLD patient cohorts, employing an in-house developed NGS (next-generation sequencing) assay. 369 subjects from 3 patient cohorts were tested, to stratify liver disease stages by fibrosis. NAFLD progression may be regulated by pathways involved in fibrosis, inflammation. endothelial blood vessel development and adaptive immunity, hepatic stallate cell activation, PI3/AKT signaling, thyroid cancer signaling, IGF-1 signaling, G2/M DNA damage checkpoint regulation, synaptogenesis signaling pathway, epithelial adherens junction signaling, molecular mechanisms of cancer, systemic lupus erythematosus in B Cell signaling, germ cell-sertoli cell junction signaling, FXR/RXR signaling, etc. In a cohort of 208 individuals, classifiers were developed to identify NAFLD, and its progressive form, NASH (non-alcoholic steatohepatitis), from non-liver diseased subjects (AUC=0.92 and 0.93, respectively). Individuals with NAFLD were distinguished from those with NASH (AUC=0.77), and the ability to stratify liver disease progression by liver fibrosis stages (early stage: F0 and F1 vs. late stage: F3 and F4) was demonstrated with an AUC value of 0.8. The fibrosis stratification was validated in prospectively collected cohorts of 96 and 65 subjects (AUC=0.83and 0.91), with high specificity and sensitivity. In some embodiments, genes comprising the fibrosis stage classifier indicate that biological processes regulating blood vessel development and immune responses may underpin the mechanisms contributing to liver fibrosis. The present disclosure includes the first use of a class of biomarkers in understanding the fatty liver disease. The systems and methods herein may enable utility of this assay to elucidate liver disease biology and to translate into research and drug discovery programs.

All studies for patient sample collection were approved by their institutional IRB (Institutional Review Boards). Serum from non-liver diseased individuals were obtained from San Diego Blood Bank. Retrospectively collected serum for the NASH individuals were obtained from the University of Indiana School of Medicine. Prospectively collected samples from NAFLD individuals were collected from the CTSI biorepositories at University of Florida and University of Indiana.

Cell-free mRNA was extracted from serum and eluted in a volume of 16 uL. 1 uL of extracted cf-mRNA was analyzed on an Agilent RNA 6000 Pico chip (Agilent Technologies, Cat. #5067-1513) to confirm successful isolation of cf-mRNA. 5 uL of the extracted cf-mRNA (Thermo Fisher Scientific, Cat. #4456740),was converted into a sequencing library. Qualitative and quantitative analysis of the NGS library preparation process was conducted using a chip-based electrophoresis and libraries were quantified using a qPCR-based quantification kit (Roche, Cat. #KK4824). Sequencing was performed on an Illumina NextSeq500 platform, using paired-end, 75-cycle sequencing.

For the Training and validation cohort, a median of 8.7 million pass-filter reads per sample (range 8.1-16.2 million reads) was sequenced. Base-calling was performed on an Illumina BaseSpace platform, using the FASTQ Generation Application. Adaptor sequences are removed and low-quality bases trimmed using cutadapt (v1.11). Reads shorter than 15 base-pairs were excluded from subsequent analysis. Read sequences greater than 15 base-pairs were to the human reference genome GRCh38 using STAR (v2.5.2b) with GENCODE v24 gene models. Duplicated reads are removed using the samtools (v1.3.1) rmdup command. Gene-expression levels were calculated from de-duplicated BAM files using RSEM (v1.3.0).

Non-negative matrix factorization (NMF) was performed to decompose normalized gene-expression profiles from cf-mRNA into 12 components. NMF decomposition uses the “decomposition.NMF” class in sciki-learn Python library, so that genes sharing similar expression patterns across samples are grouped together in an un-supervised manner. Genes with >40% loading attributable to a particular component were considered enriched in the component. For each component, genes enriched in the component were selected and the following was examined: 1) their expression levels across 51 human tissues in GTEx; 2) their expression levels across 55 human hematopoietic cell types from the Blueprint Epigenome consortium; and 3) their potential Gene Ontology functional enrichment. By integrating these data, enrichment of cell-types for a particular component/group of genes could be ascertained.

From the “Training cohort” samples, the average transcripts per million (TPM) from sample replicates was obtained. Using these gene-expression values, logistic regression model with ridge regularization was applied to diagnose NAFL liver disease samples. The LogisticRegression method with L2 regularization within the scikit-learn Python library for implementing the classification was used. Meta-parameters are determined by cross-validation repeated 15 times. During each iteration of cross-validation, 40% of the cohort as testing set was randomly withheld; classifier was built using the training set (remaining 60% of the cohort) and then applied to the testing set. Receiver operating characteristic (ROC) curves are calculated by plotting the true positive rate against the false positive rate at various threshold cutoffs. Area under the ROC curve (AUC) are calculated for each of the 15 iterations of cross-validation. Average ROC curves are calculated from these 15 cross-validations and the meta-parameter with the best average AUC was selected. For fibrosis classification, fibrosis stages 0 and 1 samples are designated as “early” stage samples while fibrosis stages 3 and 4 samples are designated as “late” stage samples. The classifier with the chosen meta-parameter to the entire “Training cohort” and applied the derived model to validation cohorts was fitted.

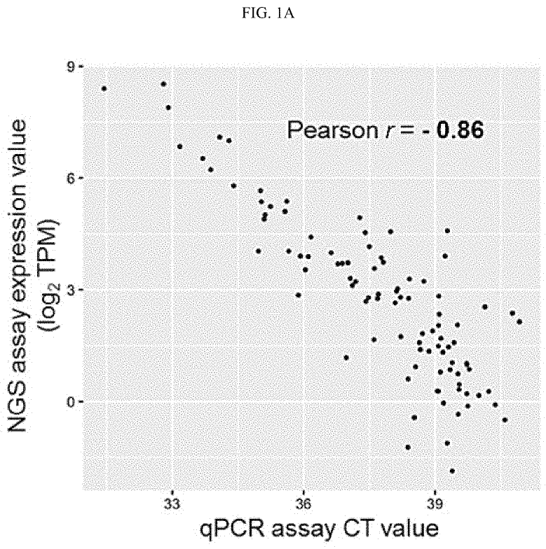

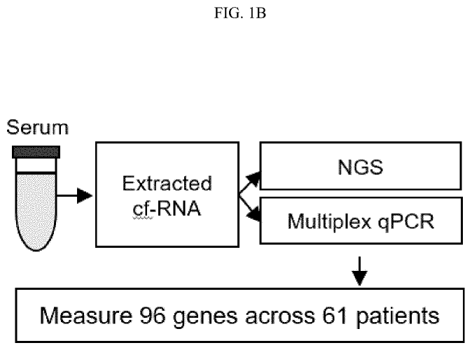

In some embodiments, the systems and methods herein provide the capability of the cf-mRNA NGS assay to accurately quantify cf-mRNA transcripts, by comparing expression data to a multiplex qPCR readout (Fluidigm BioMark™). cf-mRNA, isolated from plasma of 61 individuals, was split into aliquots for cf-mRNA NGS and multiplex qPCR profiling, to measure % genes known to be expressed in healthy or NAFLD liver tissue, Gene-expression data generated from these orthogonal assays were highly correlated (Pearson correlation, r=−0.86); high cf-mRNA TPM values correlating with low qPCR CT (cycle threshold) values (i.e., high expression) (FIGS. 1A and 1B). FIGS. 1A and 1B show technical validation of gene-expression using cf-mRNA NGS assay (at primary axis values 0, 3, 6, and 9) vs. multiplex qPCR readout (at primary axis values 33, 36, and 39). In this embodiment, each data-point represents average gene-expression of a single gene measured by the cf-mRNA NGS assay and qPCR in 61 individuals (% genes measured). Next, technical variability of the cf-mRNA NGS assay was assessed, by measuring whole-transcriptome gene-expression across 14 technical replicates of pooled cf-mRNA, extracted from serum of non-liver diseased individuals. From these studies, highly correlated expression profiles across replicates (mean log 2 TPM Pearson's correlation=0.906; range=0.8%-0.914) (Table 1) was observed. Table 1 shows Pearson correlation of gene-expression (log 2 TPM value) for genes with >0 TPM expression-level in at least 1 of 14 replicate samples. Together, these data highlight the accuracy and technical robustness of the cf-mRNA NGS assay.

TABLE 1

| | Rep# | Rep# | Rep# | Rep# | Rep# | Rep# | Rep# | Rep# | Rep# | Rep# | Rep# | Rep# | Rep# | Rep# |
|---|---|---|---|---|---|---|---|---|---|---|---|---|---|---|
| | 1 | 2 | 3 | 4 | 5 | 6 | 7 | 8 | 9 | 10 | 11 | 12 | 13 | 14 |
| Rep# 1 | 1 | 0.910 | 0.908 | 0.907 | 0.911 | 0.903 | 0.908 | 0.900 | 0.907 | 0.904 | 0.908 | 0.908 | 0.907 | 0.900 |
| Rep# 2 | | 1 | 0.910 | 0.909 | 0.910 | 0.906 | 0.910 | 0.903 | 0.911 | 0.907 | 0.911 | 0.911 | 0.908 | 0.901 |
| Rep# 3 | | | 1 | 0.907 | 0.910 | 0.903 | 0.909 | 0.902 | 0.908 | 0.907 | 0.907 | 0.907 | 0.907 | 0.901 |
| Rep# 4 | | | | 1 | 0.907 | 0.904 | 0.905 | 0.899 | 0.907 | 0.905 | 0.906 | 0.907 | 0.903 | 0.898 |
| Rep# 5 | | | | | 1 | 0.904 | 0.907 | 0.901 | 0.908 | 0.906 | 0.907 | 0.908 | 0.906 | 0.900 |
| Rep# 6 | | | | | | 1 | 0.902 | 0.898 | 0.905 | 0.903 | 0.903 | 0.905 | 0.901 | 0.896 |
| Rep# 7 | | | | | | | 1 | 0.901 | 0.907 | 0.906 | 0.907 | 0.907 | 0.904 | 0.899 |
| Rep# 8 | | | | | | | | 1 | 0.906 | 0.906 | 0.907 | 0.905 | 0.904 | 0.901 |
| Rep# 9 | | | | | | | | | 1 | 0.910 | 0.912 | 0.914 | 0.909 | 0.905 |
| Rep# 10 | | | | | | | | | | 1 | 0.912 | 0.912 | 0.911 | 0.906 |
| Rep# 11 | | | | | | | | | | | 1 | 0.913 | 0.911 | 0.905 |
| Rep# 12 | | | | | | | | | | | | 1 | 0.912 | 0.906 |
| Rep# 13 | | | | | | | | | | | | | 1 | 0.903 |
| Rep# 14 | | | | | | | | | | | | | | 1 |

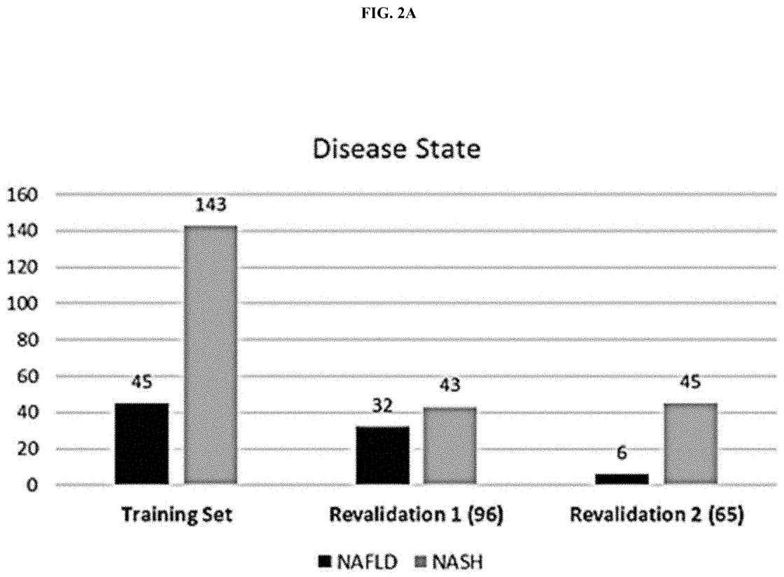

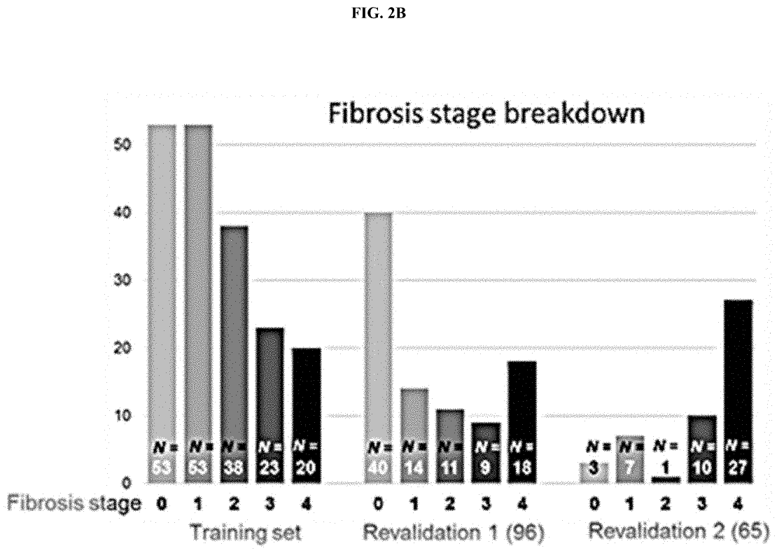

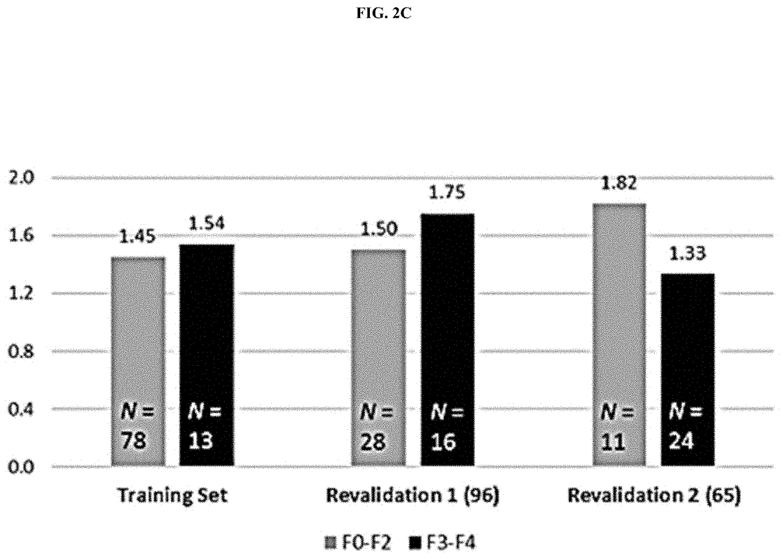

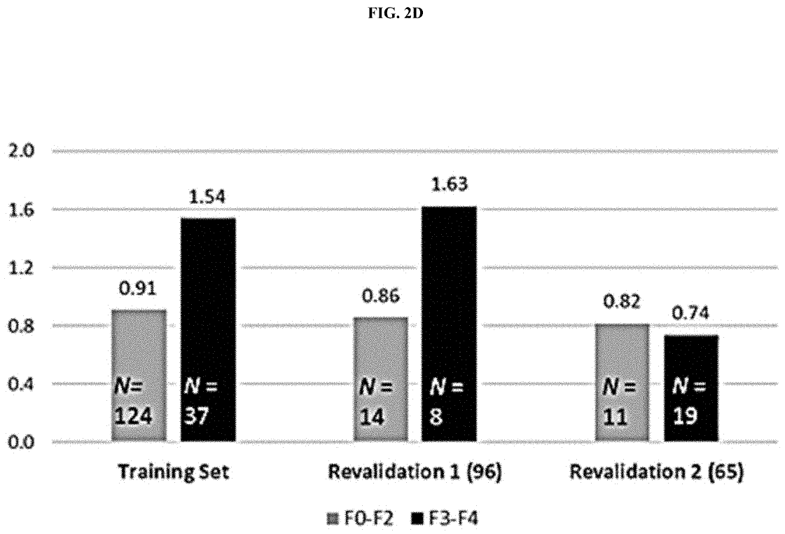

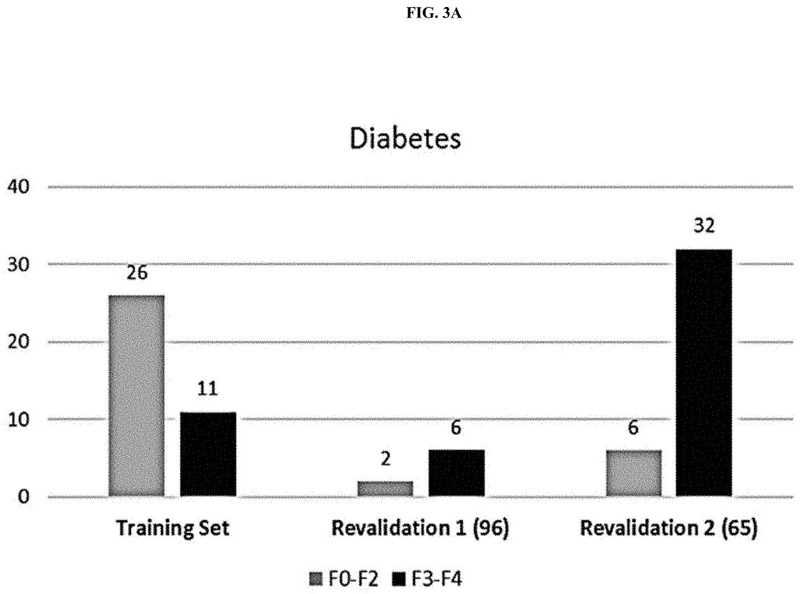

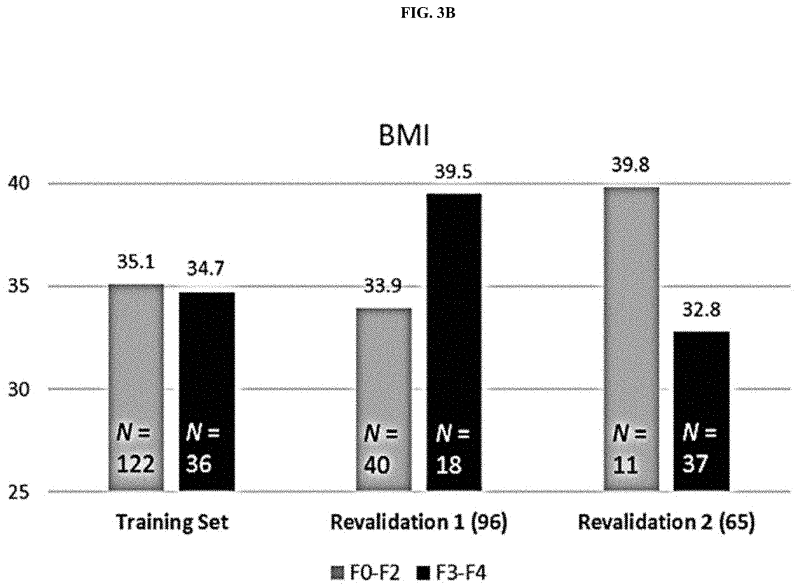

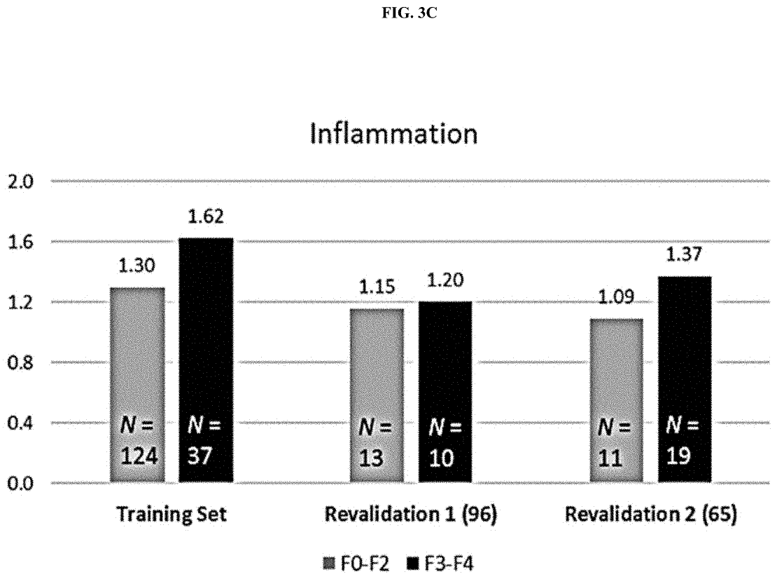

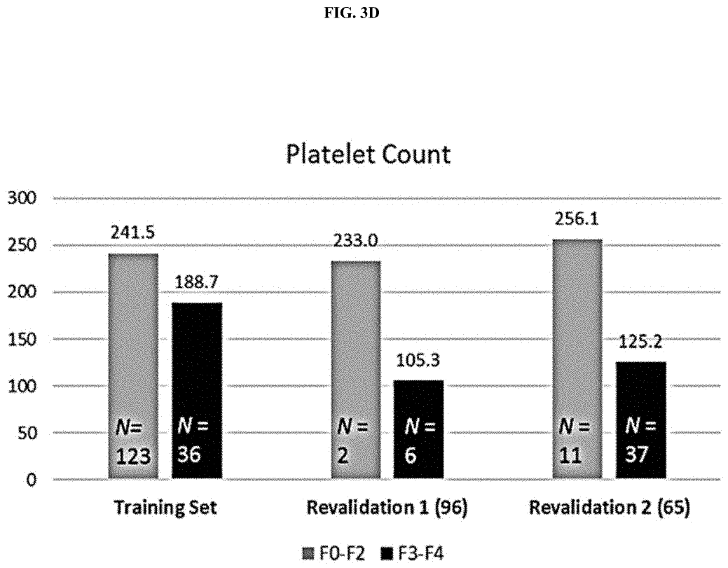

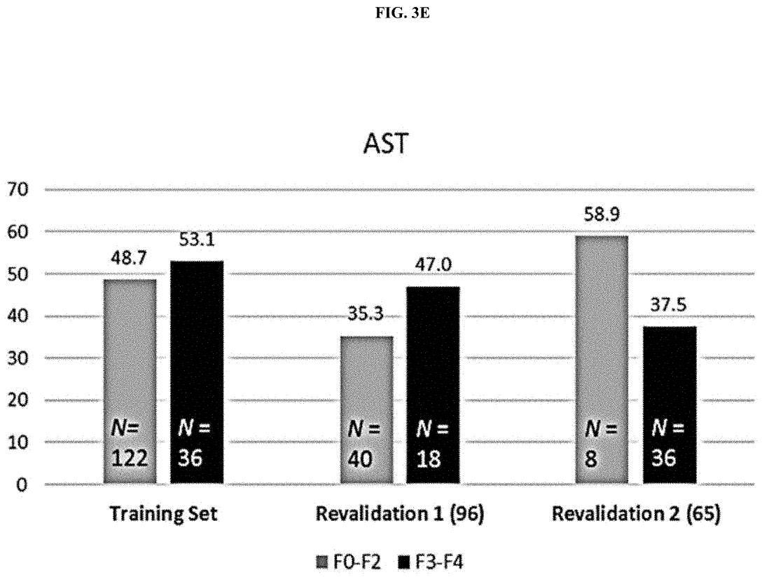

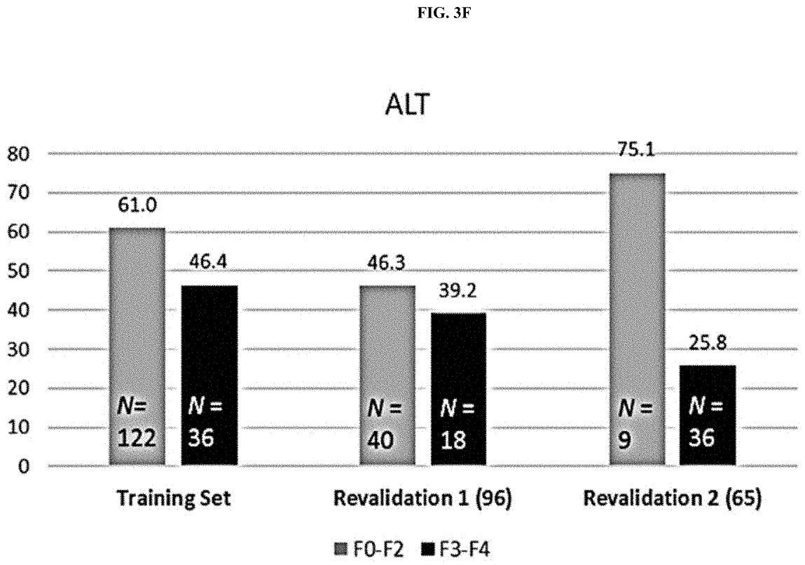

To test clinical validity of the cf-mRNA NGS profiling to diagnose and identify liver disease states of NAFLD, serum from a cohort of 208 retrospectively collected was processed, biopsy-verified samples, comprising a “Training” cohort. The liver fibrosis classifier was revalidated in 2 prospectively collected patient cohorts, “Revalidation cohort #1” (n=96 subjects) and “Revalidation cohort #2” (n=65 subjects). The key clinical parameters for subjects of the 3 study cohorts are highlighted in FIGS. 2A-D and 3A-F.

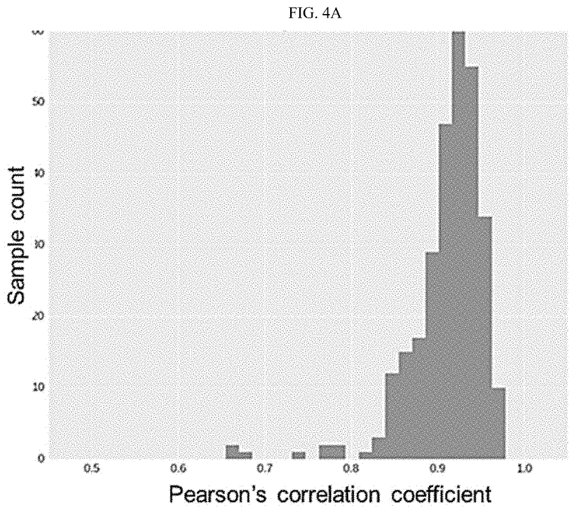

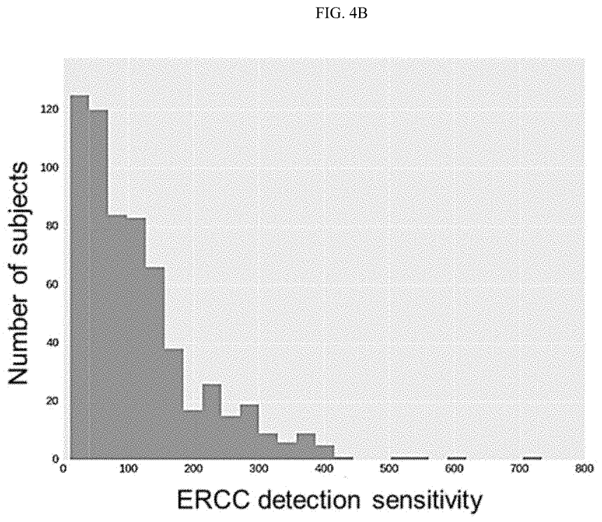

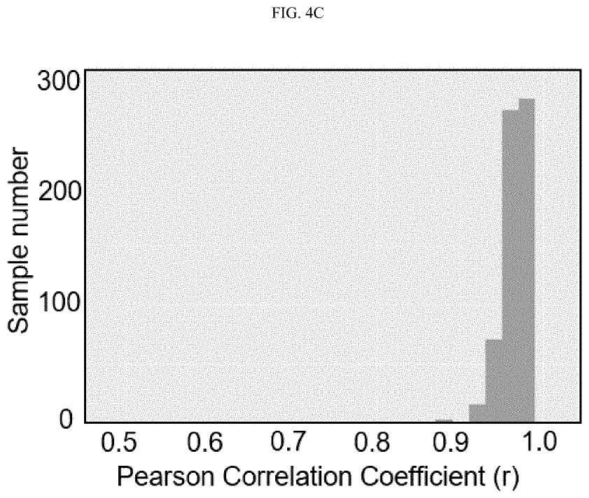

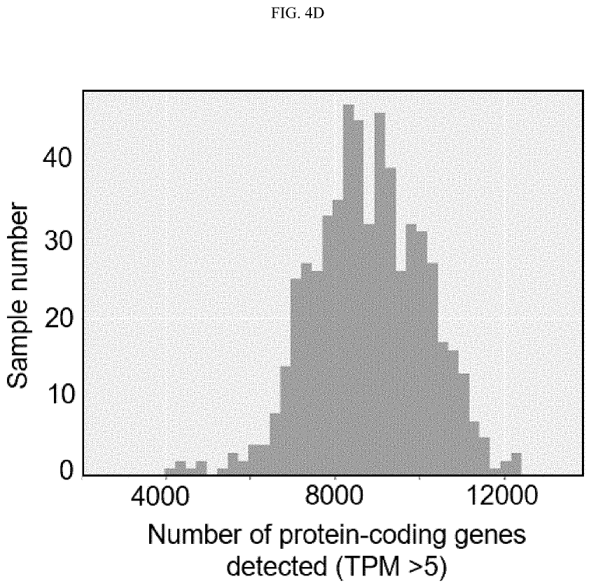

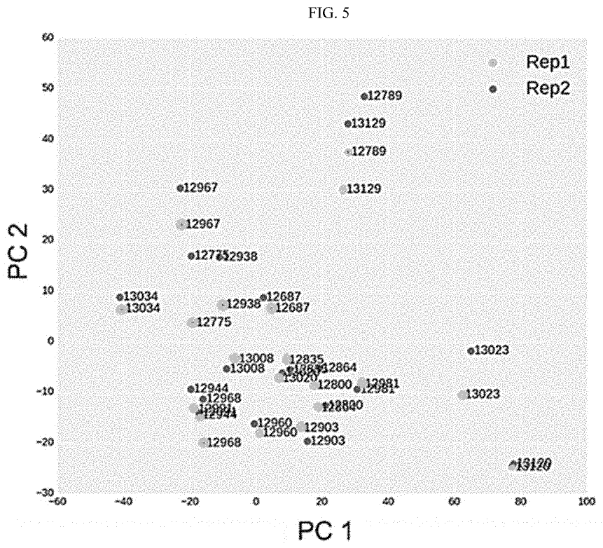

Analysis of whole-transcriptome data from technical replicates of extracted serum cf-mRNA, indicated excellent technical reproducibility of gene-expression measurement (log 2 TPM Pearson's correlation >0.9) (FIG. 4A). Furthermore, PCA analysis of cf-mRNA gene-expression data indicated greater concordance between replicates of individuals compared to inter-individual variability (FIG. 5). In these studies, excellent assay sensitivity of <100 input RNA molecules was demonstrated using external ERCC (External RNA Controls Consortium) RNA spike-in controls (FIG. 4C). Taken together, these data represent the excellent technical performance of the cf-mRNA gene-expression assay.

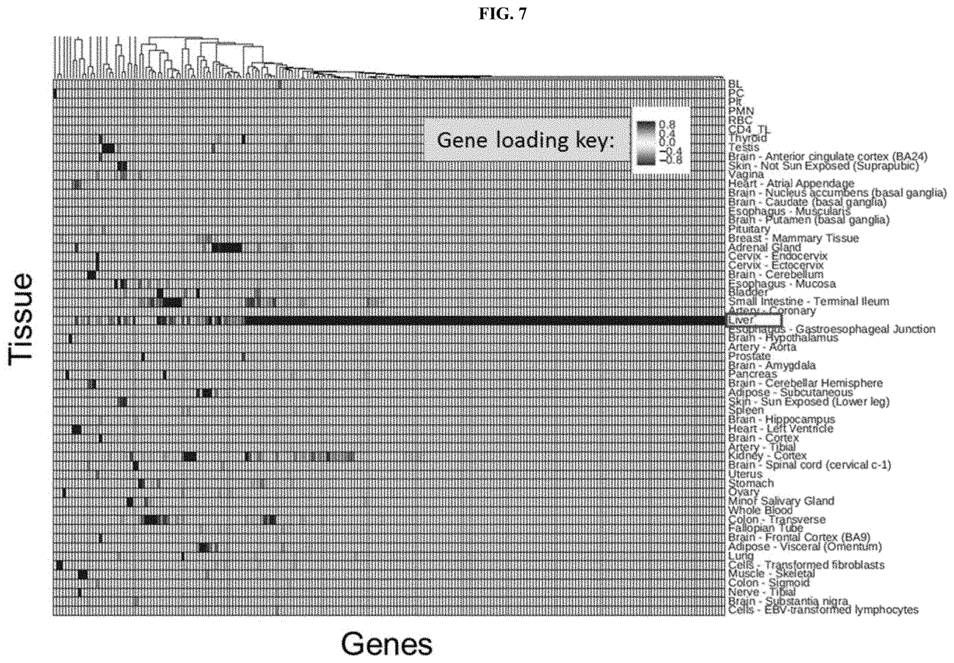

In some embodiments, cf-mRNA expression profiles are employed to diagnose and stratify NAFLD. In some embodiments, NMF (non-negative matrix factorization) is applied to extract biologically relevant information from complex gene-expression datasets. Applying NMF to the cf-mRNA expression profiles to subjects comprising the Training and validation #1 cohort, 12 co-expressed components is identified. As expected, by cross-referencing to the GTEx database, several of these NMF-derived components to contain genes highly enriched in RNA-signatures associated with blood-cells was identified, e.g., RBC (red blood cells), PMN (polymorphonuclear leukocytes) and platelets (data not shown. A component highly enriched in liver-specific transcripts (component 6) was identified (FIG. 7A and Table 2). A component highly enriched in hepatic fibrosis genes was identified FIG. 6D). This component correlated with fibrosis stage determined by liver biopsy. A component enriched in inflammatory and endothelial genes was identified (FIG. 6D). This component correlated with liver inflammation determined by liver biopsy.

TABLE 2

| # | Ensembl ID | Gene symbol | Loading fraction |
|---|---|---|---|
| 1 | ENSG00000109181 | UGT2B10 | 1.000 |
| 2 | ENSG00000113889 | KNG1 | 1.000 |
| 3 | ENSG00000113905 | HRG | 1.000 |
| 4 | ENSG00000116785 | CFHR3 | 1.000 |
| 5 | ENSG00000132855 | ANGPTL3 | 1.000 |
| 6 | ENSG00000138823 | MTTP | 1.000 |
| 7 | ENSG00000158104 | HPD | 1.000 |
| 8 | ENSG00000163825 | RTP3 | 1.000 |
| 9 | ENSG00000171557 | FGG | 1.000 |
| 10 | ENSG00000171560 | FGA | 1.000 |
| 11 | ENSG00000171564 | FGB | 1.000 |
| 12 | ENSG00000224916 | APOC4-APOC2 | 1.000 |
| 13 | ENSG00000244414 | CFHR1 | 1.000 |
| 14 | ENSG00000273259 | SERPINA3 | 1.000 |
| 15 | ENSG00000276490 | RP11-400G3.5 | 1.000 |
| 16 | ENSG00000276911 | RP4-608O15.3 | 1.000 |
| 17 | ENSG00000196620 | UGT2B15 | 0.998 |
| 18 | ENSG00000080910 | CFHR2 | 0.998 |
| 19 | ENSG00000214274 | ANG | 0.998 |
| 20 | ENSG00000072080 | SPP2 | 0.996 |
| 21 | ENSG00000105697 | HAMP | 0.994 |
| 22 | ENSG00000145826 | LECT2 | 0.994 |
| 23 | ENSG00000170099 | SERPINA6 | 0.993 |
| 24 | ENSG00000080618 | CPB2 | 0.993 |
| 25 | ENSG00000180432 | CYP8B1 | 0.989 |
| 26 | ENSG00000132703 | APCS | 0.989 |
| 27 | ENSG00000157131 | C8A | 0.988 |
| 28 | ENSG00000138207 | RBP4 | 0.988 |
| 29 | ENSG00000216588 | IGSF23 | 0.986 |
| 30 | ENSG00000111700 | SLCO1B3 | 0.986 |
| 31 | ENSG00000148702 | HABP2 | 0.986 |
| 32 | ENSG00000261221 | ZNF865 | 0.985 |
| 33 | ENSG00000113600 | C9 | 0.984 |
| 34 | ENSG00000114771 | AADAC | 0.982 |
| 35 | ENSG00000160097 | FNDC5 | 0.981 |
| 36 | ENSG00000117601 | SERPINC1 | 0.979 |
| 37 | ENSG00000158874 | APOA2 | 0.973 |
| 38 | ENSG00000101981 | F9 | 0.971 |
| 39 | ENSG00000228278 | ORM2 | 0.969 |
| 40 | ENSG00000084674 | APOB | 0.969 |
| 41 | ENSG00000138109 | CYP2C9 | 0.969 |
| 42 | ENSG00000148965 | SAA4 | 0.968 |
| 43 | ENSG00000129965 | INS-IGF2 | 0.967 |
| 44 | ENSG00000131482 | G6PC | 0.963 |
| 45 | ENSG00000145192 | AHSG | 0.962 |
| 46 | ENSG00000151365 | THRSP | 0.961 |
| 47 | ENSG00000079557 | AFM | 0.960 |
| 48 | ENSG00000099937 | SERPIND1 | 0.960 |
| 49 | ENSG00000117594 | HSD11B1 | 0.957 |
| 50 | ENSG00000106927 | AMBP | 0.957 |

Table 2 shows the top 50 genes of liver-specific NMF-derived component 6 from cf-mRNA.

Closer examination of genes in NMF component 6, revealed an increased number of liver-specific genes detected in cf-mRNA of NASH (average of 113 genes) vs. NAFLD (average of 85 genes) individuals, compared to non-liver diseased controls (average of 64 genes), (P<0.001). These analyses also indicated a significantly greater number of cf-mRNA liver-specific genes identified in NASH compared to NAFLD (P=0.013) (FIG. 7B), thus demonstrating the presence of cf-mRNA liver-specific genes to be correlated with liver disease severity. From top to bottom, the vertical axis of FIG. 7A reads BL, PC, Plt, PMN, RBC, CD4_TL, Thyroid, Testis, Brain-Anterior Cingulate Cortex (BA24), Skin-Not Sun Exposed (Suprapubic), Vagina, Heart—Atrial Appendage, Brain-Nucleus Acumens (Basal Ganglia), Brain—Caudate (Basal Ganglia), Esophagus—Muscularis, Brain—Putamen (Basal Ganglia), Pituitary, Breast—Mammary Tissue, Adrenal Gland, Cervix—Endocervix, Cervix—Ectocervix, Brain—Cerebellum, Esophagus—Mucosa, Bladder, Small Intestine—Terminal Ileum, Artery—Coronary, Liver (highlighted), Esophagus—Gastroesosophageal Junction, Brain—Hypothalamus, Artery—Aorta, Prostate, Brain—Amygdala, Pancrease, Brain—Cerebellar Hemisphere, Adipose—Subcutaneous, Skin—Sun Exposed (Lower Leg), Spleen, Brain—Hippocampus, Heart—Left Ventricle, Brain—Cortex, Artery—Tibial, Kidney—Cortex, Brain—Spinal Cord (Cervical C-1), Uterus, Stomach, Ovary, Minor Salivary Gland, Whole Blood, Colon—Transverse, Fallopian Tube, Brain—Frontal Cortex (BA9), Adipose—Visceral (Omentum), Lung, Cells—Transformed Fibroblasts, Muscle—Skeletal, Colon—Sigmoid, Nerve 0—Tibial, Brain—Substrantia Nigra, and Cells—EBV-Transformed Lymphocytes.

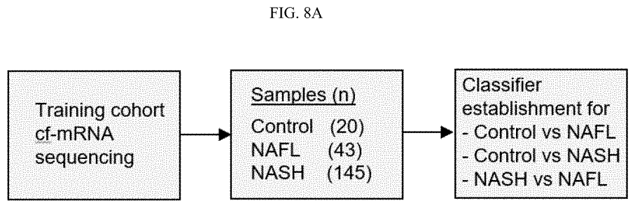

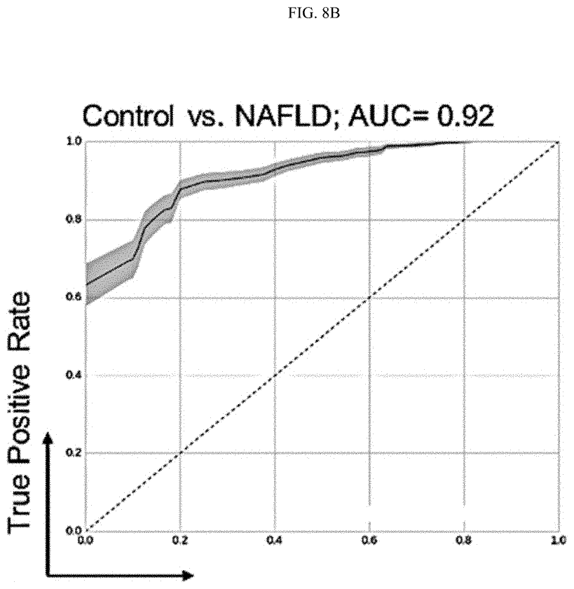

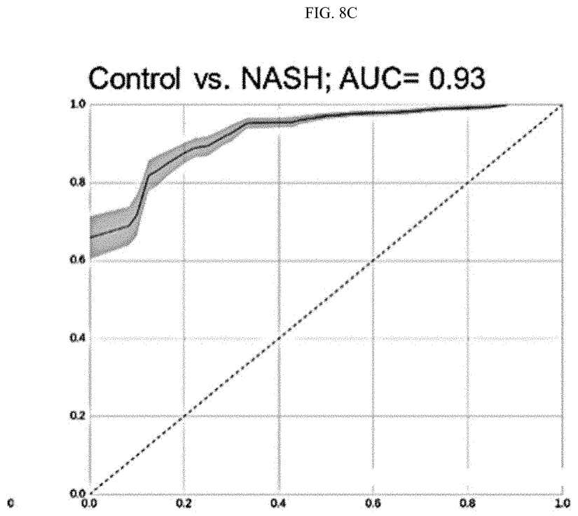

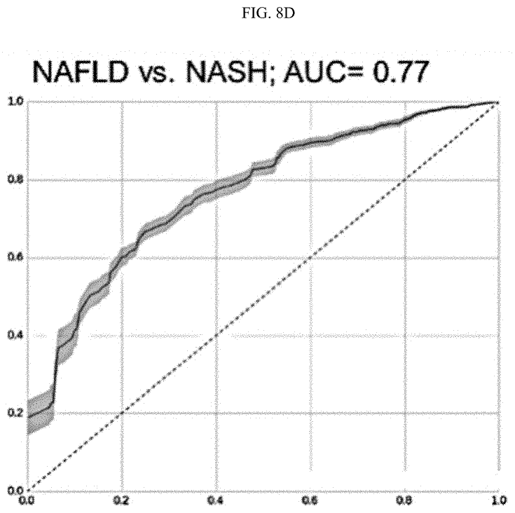

Next, cf-mRNA gene expression profiles were used to diagnose NAFL-related liver disease states. A logistic regression model was used to classify NAFLD using cf-mRNA gene-expression profiles from the Training cohort. Briefly, samples in the cohort were randomly segregated into training and testing sets. The classifier was trained on the training test data, from which ROC (Receiver Operating Characteristic) curves and associated AUC (Area Under Curve) values were calculated using testing set data. From these studies, the ability to diagnose NAFLD and NASH with AUC values of 0.92 and 0.93, was demonstrated respectively. Furthermore, stratification of NAFLD from NASH was achieved with an AUC value of 0.77 (FIGS. 8B-D). In this embodiment, shaded regions represent AUC standard error, generated from iterative cross-validation. Genes with the 50 highest coefficient values in the liver disease classification models are listed in Tables 3-6.

TABLE 3
Non-diseased vs NAFLD

| # | Gene name | Ensembl Gene ID |
|---|---|---|
| 1 | BCAS3 | ENSG00000141376 |
| 2 | CR1 | ENSG00000203710 |
| 3 | TRMT112 | ENSG00000173113 |
| 4 | KIZ | ENSG00000088970 |
| 5 | HAUS3 | ENSG00000214367 |
| 6 | TMEM64 | ENSG00000180694 |
| 7 | DDX3X | ENSG00000215301 |
| 8 | MYL4 | ENSG00000198336 |
| 9 | PPP3CA | ENSG00000138814 |
| 10 | TPD52 | ENSG00000076554 |
| 11 | CDR1-AS | — |
| 12 | CRYBG3 | ENSG00000080200 |
| 13 | WDR81 | ENSG00000276021 |
| 14 | EIF4G1 | ENSG00000114867 |
| 15 | AQP3 | ENSG00000165272 |
| 16 | APOA1 | ENSG00000118137 |
| 17 | INSR | ENSG00000171105 |
| 18 | PLPP3 | ENSG00000162407 |
| 19 | NAA20 | ENSG00000173418 |
| 20 | HP | ENSG00000257017 |
| 21 | HECTD4 | ENSG00000173064 |
| 22 | ST6GALNAC4 | ENSG00000136840 |
| 23 | TUSC2 | ENSG00000114383 |
| 24 | TRDMT1 | ENSG00000107614 |
| 25 | KIFAP3 | ENSG00000075945 |
| 26 | ADAM19 | ENSG00000135074 |
| 27 | CALM2 | ENSG00000143933 |
| 28 | RBFOX2 | ENSG00000100320 |
| 29 | NR1D1 | ENSG00000126368 |
| 30 | PDE4A | ENSG00000065989 |
| 31 | SLC25A38 | ENSG00000144659 |
| 32 | NDFIP1 | ENSG00000131507 |
| 33 | ZC3HAV1 | ENSG00000105939 |
| 34 | AQP1 | ENSG00000240583 |
| 35 | CELF2 | ENSG00000048740 |
| 36 | CLSPN | ENSG00000092853 |
| 37 | ZNF333 | ENSG00000160961 |
| 38 | SMU1 | ENSG00000122692 |
| 39 | LY86 | ENSG00000112799 |
| 40 | TOX | ENSG00000198846 |
| 41 | ALB | ENSG00000163631 |
| 42 | RNF123 | ENSG00000164068 |
| 43 | ALAS2 | ENSG00000158578 |
| 44 | CCNI | ENSG00000118816 |
| 45 | ZMAT3 | ENSG00000172667 |
| 46 | MAP4K4 | ENSG00000071054 |
| 47 | ZCCHC3 | ENSG00000247315 |
| 48 | PER1 | ENSG00000179094 |
| 49 | SLFN11 | ENSG00000172716 |
| 50 | BET1L | ENSG00000177951 |

TABLE 4
Non-diseased vs NASH

| # | Gene name | Ensembl Gene ID |
|---|---|---|
| 1 | APOH | ENSG00000091583 |
| 2 | XBP1 | ENSG00000100219 |
| 3 | DHX38 | ENSG00000140829 |
| 4 | ETFB | ENSG00000105379 |
| 5 | GCOM1 | ENSG00000137878 |
| 6 | HSPG2 | ENSG00000142798 |
| 7 | SAMD4A | ENSG00000020577 |
| 8 | CSTB | ENSG00000160213 |
| 9 | VAT1 | ENSG00000108828 |
| 10 | VAMP3 | ENSG00000049245 |
| 11 | POLD4 | ENSG00000175482 |
| 12 | USP31 | ENSG00000103404 |
| 13 | SLFN14 | ENSG00000236320 |
| 14 | ALDOB | ENSG00000136872 |
| 15 | FAM195B | — |
| 16 | DDX39A | ENSG00000123136 |
| 17 | ACO2 | ENSG00000100412 |
| 18 | CUL4A | ENSG00000139842 |
| 19 | FN1 | ENSG00000115414 |
| 20 | SEPN1 | — |
| 21 | APOB | ENSG00000084674 |
| 22 | MT2A | ENSG00000125148 |
| 23 | EIF2D | ENSG00000143486 |
| 24 | NAP1L4 | ENSG00000205531 |
| 25 | DRG1 | ENSG00000185721 |
| 26 | KLHL5 | ENSG00000109790 |
| 27 | SGK1 | ENSG00000118515 |
| 28 | RPS13 | ENSG00000110700 |
| 29 | NDUFB1 | ENSG00000183648 |
| 30 | GRB10 | ENSG00000106070 |
| 31 | LBR | ENSG00000143815 |
| 32 | MRPL41 | ENSG00000182154 |
| 33 | PTBP3 | ENSG00000119314 |
| 34 | SDHC | ENSG00000143252 |
| 35 | ALOX5 | ENSG00000275565 |
| 36 | ARHGAP35 | ENSG00000160007 |
| 37 | REV3L | ENSG00000009413 |
| 38 | VWF | ENSG00000110799 |
| 39 | HIST1H4I | ENSG00000276180 |
| 40 | TNS2 | ENSG00000111077 |
| 41 | UQCRQ | ENSG00000164405 |
| 42 | DNASE1L3 | ENSG00000163687 |
| 43 | NCL | ENSG00000115053 |
| 44 | RAB11B | ENSG00000185236 |
| 45 | SGTA | ENSG00000104969 |
| 46 | CDC37 | ENSG00000105401 |
| 47 | CAPG | ENSG00000042493 |
| 48 | PRR14L | ENSG00000183530 |
| 49 | ZFAND6 | ENSG00000086666 |
| 50 | FGL2 | ENSG00000127951 |

TABLE 5
NAFL vs NASH

| # | Gene name | Ensembl Gene ID |
|---|---|---|
| 1 | OAS2 | ENSG00000111335 |
| 2 | AKR1A1 | ENSG00000117448 |
| 3 | PGK1 | ENSG00000102144 |
| 4 | CCDC50 | ENSG00000152492 |
| 5 | POLR2C | ENSG00000102978 |
| 6 | MLF2 | ENSG00000089693 |
| 7 | ALDH2 | ENSG00000111275 |
| 8 | RABIF | ENSG00000183155 |
| 9 | MCFD2 | ENSG00000180398 |
| 10 | B3GNT8 | ENSG00000177191 |
| 11 | AAK1 | ENSG00000115977 |
| 12 | BAK1 | ENSG00000030110 |
| 13 | GCA | ENSG00000115271 |
| 14 | BTBD9 | ENSG00000183826 |
| 15 | SAFB2 | ENSG00000130254 |
| 16 | KIFC3 | ENSG00000140859 |
| 17 | PRDX6 | ENSG00000117592 |
| 18 | LRRC4 | ENSG00000128594 |
| 19 | ZNF426 | ENSG00000130818 |
| 20 | VASH1 | ENSG00000071246 |
| 21 | PDE8A | ENSG00000073417 |
| 22 | KIZ | ENSG00000088970 |
| 23 | HBA2 | ENSG00000188536 |
| 24 | ZCCHC9 | ENSG00000131732 |
| 25 | AHNAK | ENSG00000124942 |
| 26 | PRMT7 | ENSG00000132600 |
| 27 | STT3A | ENSG00000134910 |
| 28 | FAM213A | ENSG00000122378 |
| 29 | NUDT9 | ENSG00000170502 |
| 30 | TPGS2 | ENSG00000134779 |
| 31 | SELPLG | ENSG00000110876 |
| 32 | DHRS13 | ENSG00000167536 |
| 33 | MACF1 | ENSG00000127603 |
| 34 | TBC1D22B | ENSG00000065491 |
| 35 | RIOK3 | ENSG00000101782 |
| 36 | MOSPD3 | ENSG00000106330 |
| 37 | MET | ENSG00000105976 |
| 38 | PNPO | ENSG00000108439 |
| 39 | TYK2 | ENSG00000105397 |
| 40 | IKZF3 | ENSG00000161405 |
| 41 | SHQ1 | ENSG00000144736 |
| 42 | PKP4 | ENSG00000144283 |
| 43 | C16orf62 | ENSG00000103544 |
| 44 | AKAP13 | ENSG00000170776 |
| 45 | UBE2Z | ENSG00000159202 |
| 46 | SLC15A3 | ENSG00000110446 |
| 47 | DCAF12 | ENSG00000198876 |
| 48 | SERPINB9 | ENSG00000170542 |
| 49 | CDK4 | ENSG00000135446 |
| 50 | KNG1 | ENSG00000113889 |

TABLE 6
Early vs Late Fibrosis

| # | Gene name | Ensembl Gene ID |
|---|---|---|
| 1 | IGF2 | ENSG00000284779 |
| 2 | FN3K | ENSG00000167363 |
| 3 | MLLT4 | — |
| 4 | TCF7L2 | ENSG00000148737 |
| 5 | FSCN1 | ENSG00000075618 |
| 6 | MYO10 | ENSG00000145555 |
| 7 | KALRN | ENSG00000160145 |
| 8 | KCNA3 | ENSG00000177272 |
| 9 | GNA12 | ENSG00000146535 |
| 10 | PLVAP | ENSG00000130300 |
| 11 | LEF1 | ENSG00000138795 |
| 12 | RDX | ENSG00000137710 |
| 13 | CLPTM1L | ENSG00000274811 |
| 14 | SLC9A1 | ENSG00000090020 |
| 15 | FRMD3 | ENSG00000172159 |
| 16 | BTBD6 | ENSG00000184887 |
| 17 | TPTEP1 | ENSG00000100181 |
| 18 | SLC2A1 | ENSG00000117394 |
| 19 | MTSS1L | ENSG00000132613 |
| 20 | PLEKHA4 | ENSG00000105559 |
| 21 | STARD3 | ENSG00000131748 |
| 22 | TOB1 | ENSG00000141232 |
| 23 | HECW2 | ENSG00000138411 |
| 24 | TRAF3IP1 | ENSG00000204104 |
| 25 | NDFIP1 | ENSG00000131507 |
| 26 | ATXN1L | ENSG00000224470 |
| 27 | BCL11B | ENSG00000127152 |
| 28 | MTMR2 | ENSG00000087053 |
| 29 | NUTF2 | ENSG00000102898 |
| 30 | C16orf62 | — |
| 31 | CTNNA1 | ENSG00000044115 |
| 32 | PPP1R14B | ENSG00000173457 |
| 33 | ZNF362 | ENSG00000160094 |
| 34 | ZNF358 | ENSG00000198816 |
| 35 | SCAP | ENSG00000114650 |
| 36 | MPST | ENSG00000128309 |
| 37 | PFKL | ENSG00000141959 |
| 38 | TSTA3 | ENSG00000104522 |
| 39 | MYCT1 | ENSG00000120279 |
| 40 | LIMCH1 | ENSG00000064042 |
| 41 | SHANK3 | ENSG00000251322 |
| 42 | RABGEF1 | ENSG00000154710 |
| 43 | ARHGEF18 | ENSG00000104880 |
| 44 | PDE2A | ENSG00000186642 |
| 45 | MAP4K1 | ENSG00000104814 |
| 46 | SNX8 | ENSG00000106266 |
| 47 | ARRDC2 | ENSG00000105643 |
| 48 | TBC1D9 | ENSG00000109436 |
| 49 | CYP2E1 | ENSG00000130649 |
| 50 | PITPNM3 | ENSG00000091622 |

Several of these genes represent canonical liver-specific transcripts, e.g., Albumin, Apolipoprotein B, and those listed in Table 2.

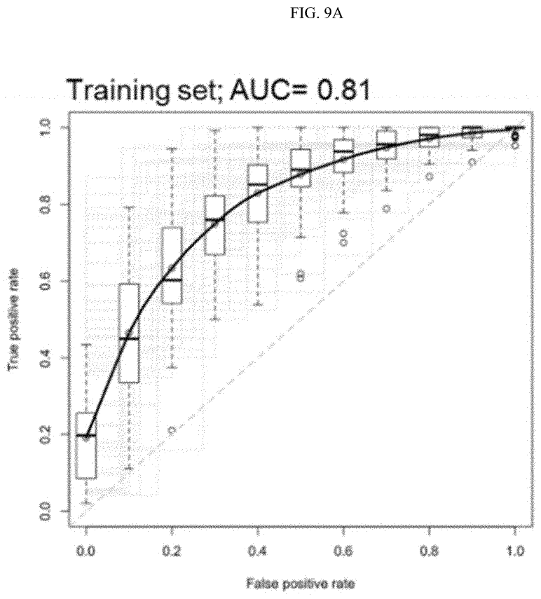

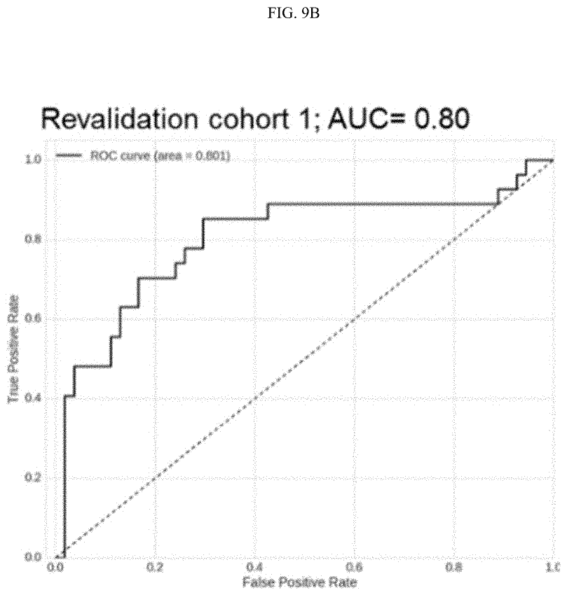

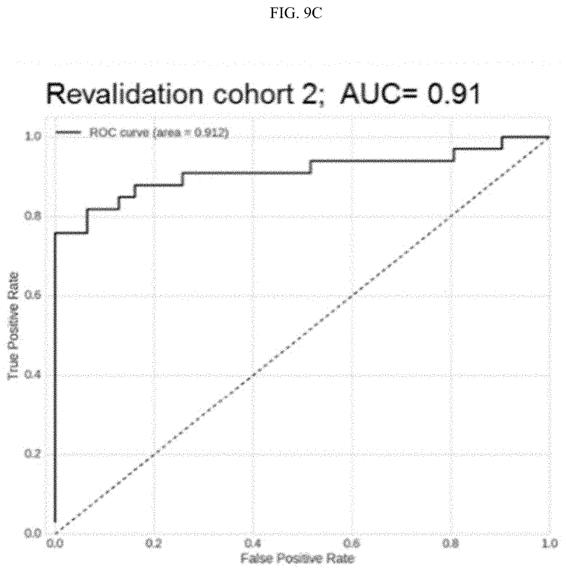

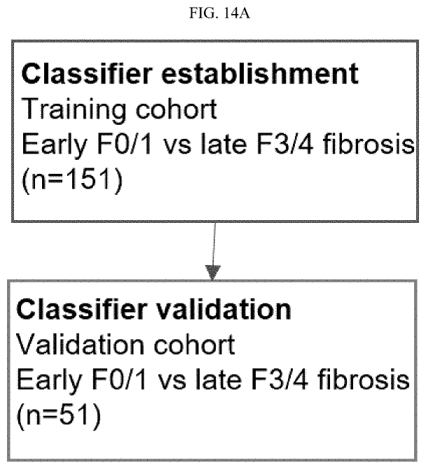

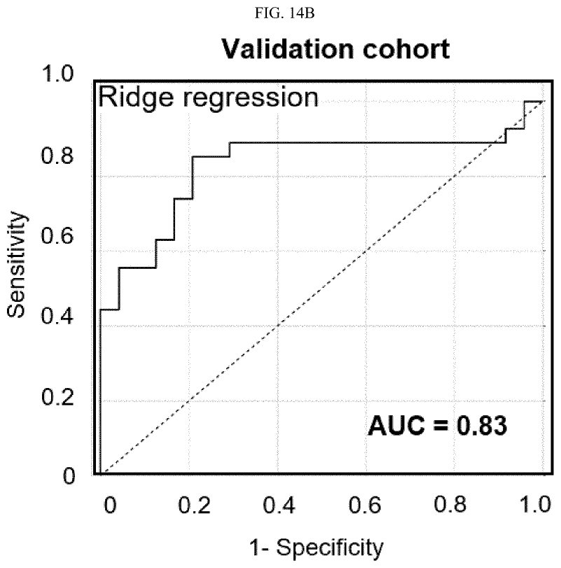

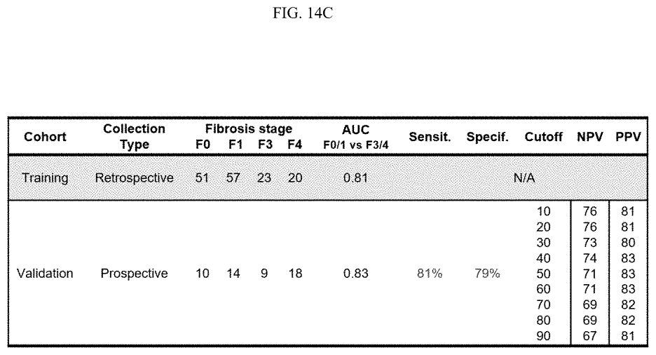

Identifying stages of fibrosis is useful in NAFLD clinical management. Using the Training cohort samples, the ability of the assay to stratify fibrosis stages was tested. The resulting classifier was able to stratify non-liver diseased individuals from “advanced” fibrosis (F3, F4), with an AUC value of 0.92. To stratify within fibrosis stages, comparison of “early” (F0, F1) vs. “advanced” (F3, F4) fibrosis subjects (excluding intermediate F2 individuals), resulting in a classification AUC value of 0.81 (FIG. 14C, FIGS. 9A-9C). Genes with the 50 largest coefficient values for fibrosis differentiation are listed in Tables 3-6. To further test utility of the classifier in independent patient cohorts, fibrosis classification with AUC values of 0.80 and 0.91, in Revalidation cohorts #1 and #2 respectively (FIG. 14C, FIGS. 9A-9C) was demonstrated. FIG. 14C shows exemplary cohort characteristics and AUC values for stratifying NAFLD related fibrosis stages using cf-mRNA gene-expression profiles.

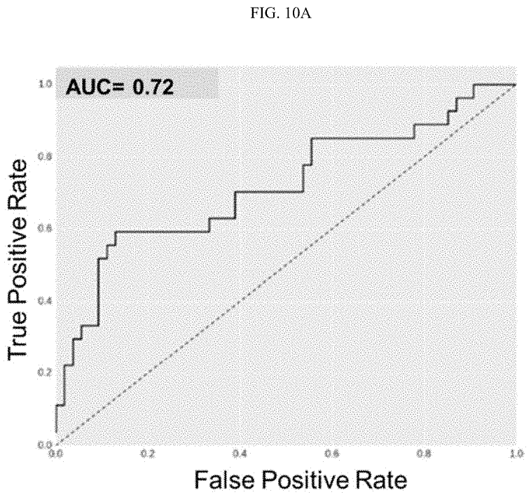

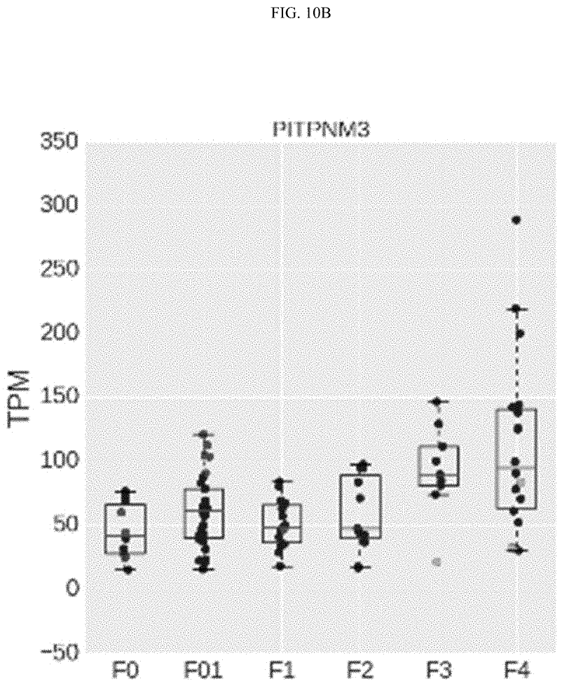

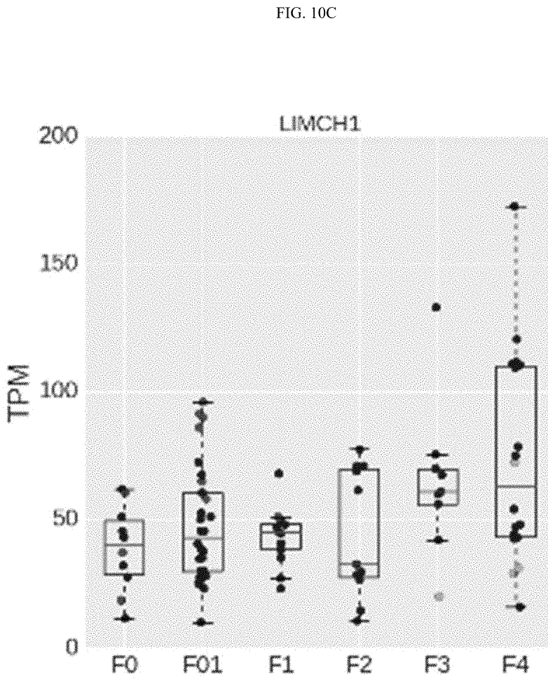

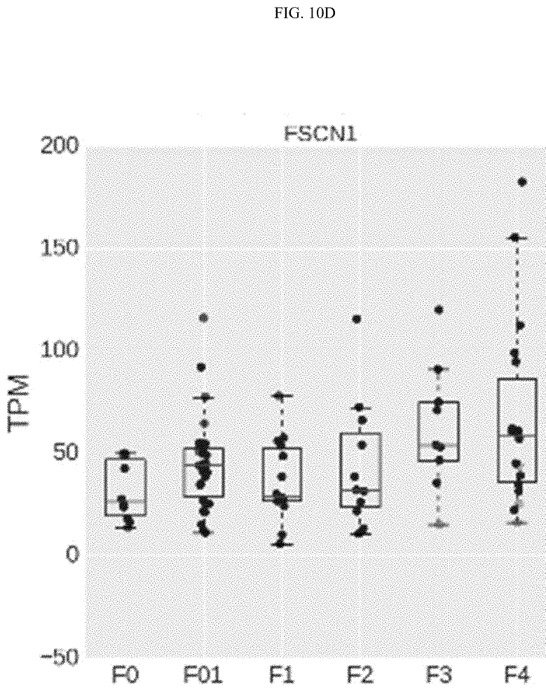

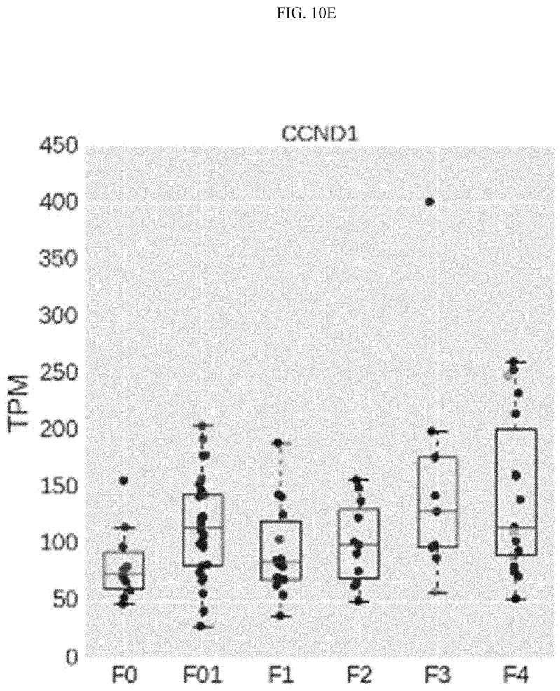

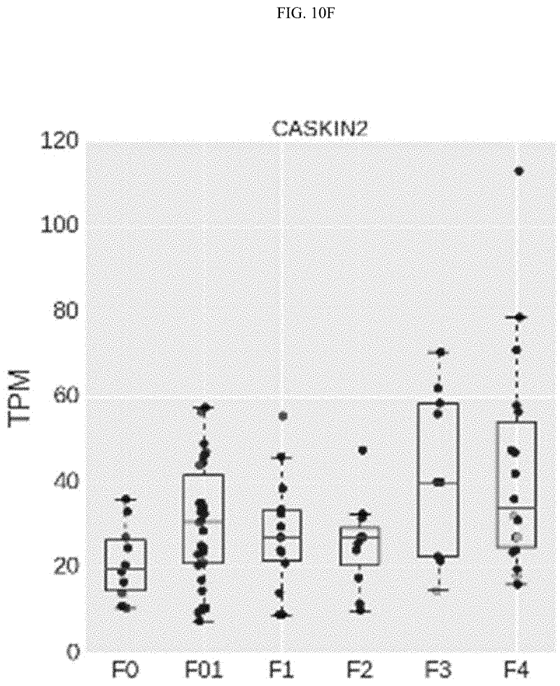

To understand the minimal number of classifier genes required to stratify fibrosis stages, five genes with cf-mRNA gene-expression positively correlated with fibrosis stages were identified (PITPNM2, LIMCH1, FSCN1, CCND1, and CASKIN2). A linear combination model of cf-mRNA gene-expression profiles of these genes could stratify “early” and “advanced” fibrosis stages with an AUC value of 0.72 in the Training cohort samples (FIGS. 10A-10F).

These studies not only demonstrate the robustness of the cf-mRNA liver fibrosis classifier, but also indicate the ability to stratify liver fibrosis using cf-mRNA gene-expression using a small panel of target genes.

Also assessed were parameters associated with the NAFLD liver disease classifier relevant to clinical diagnoses. For fibrosis stratification a specificity level of 80% was achieved with an assay sensitivity of 70% and 85%, in Revalidation cohorts 1 and 2 respectively. As the range of referral rate varies considerably between medical centers, the positive- and negative-predictive values (PPV and NPV) of the assay over a range of patient referral rates were tested, and are reported in FIG. 14B. Of particular note, the excellent NPV of the cf-mRNA assay for liver fibrosis stratification was demonstrated. FIG. 14B shows PPV and NPV of the liver fibrosis classifier in 2 prospectively collected NALF cohorts, at varying assay sensitivity and specificity thresholds.

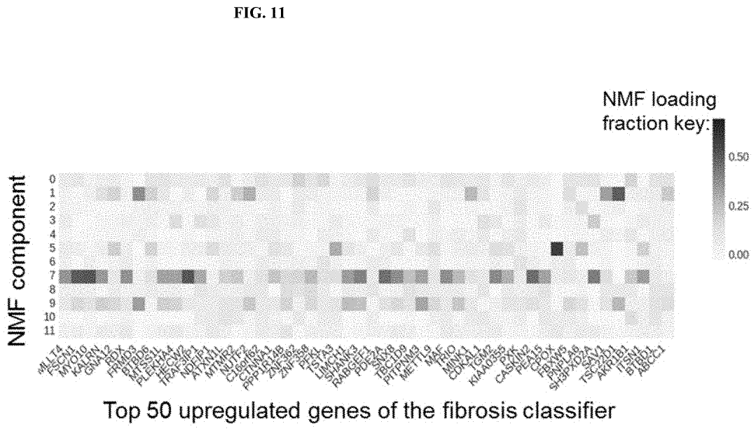

Next, the biologically relevant pathways were sought to identify the biologically relevant pathways associated with the NAFLD classifier. Employing NMF to identify groups of co-expressed genes from cf-mRNA data from the Training cohort and Revalidation 1 cohort, it was demonstrated that the majority of genes with high predictive power in the liver fibrosis classifier (Tables 3-6), were enriched in NMF-derived component 10 (Table 11, FIG. 11).

Table 11 and FIG. 11 show the top 50 genes with highest Loading fractions in NMF-derived component 10, whereby the top 50 genes (from left to right) are MLLT4, FSCN1, MYO10, GNA12, RDX, FRMD3, BTBD6, MTSS1L, PLEKHA4, HECW2, TRAF3IP1, NDFIP1, ATXN1L, MTMR2, NUTF2, C16orf62, CTNNA1, PPP1R14B, ZNF362, ZNF358, PFKL, TSTA3, LIMCH1, SHANK3, RABGEF1, PDE2A, SNX8, TBC1D9, PITPNM3, METTL9, MAF, TRIO, MINK1, CKDAL1, TGM2, KIAA0355, PXK, CASKIN2, PEA15, CPOX, FBXW5, PNPLA6, SH3PXD2A, SAV1, TSC22D1, AKR1B1, ITSN1, BTBD1, and ABCC1.

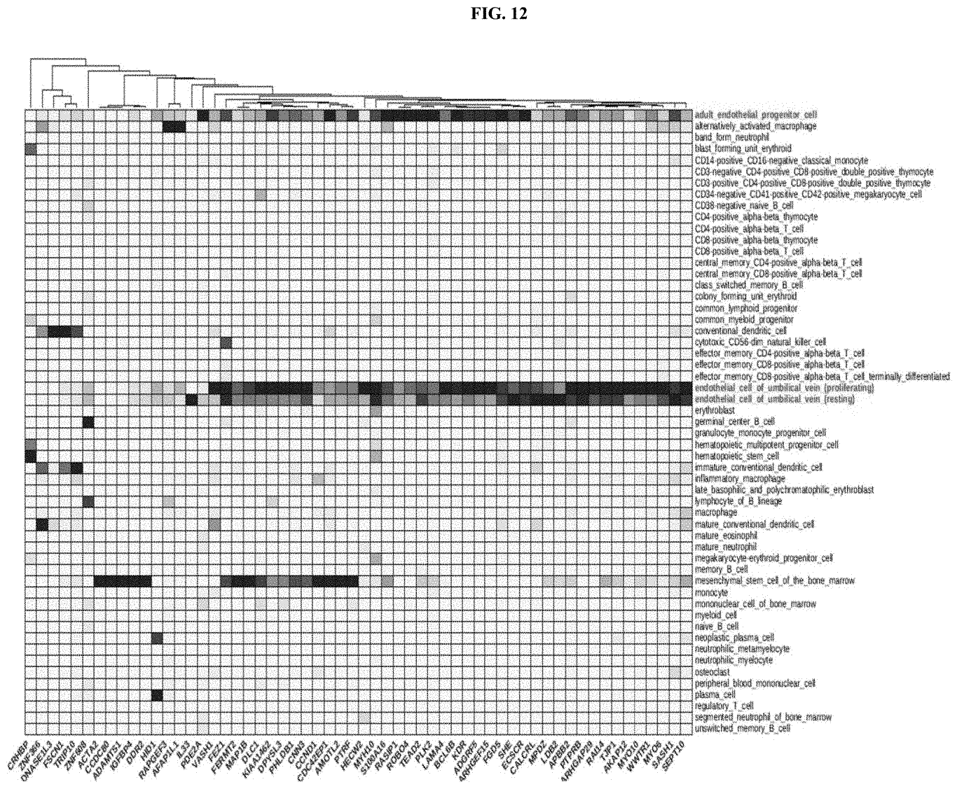

Using GO Enrichment Analysis and the Blueprint RNA-seq database, it was shown that the genes in this component are highly expressed in endothelial cells and enriched in functional categories related to blood vessel development, vasculature, and angiogenic processes (FIG. 12).

Per FIG. 12, the genes from left to right are CRHBP, ZNF366, DNASE1L3, FSCN1, TRIP10, ZN608, ACTA2, CCDC80, ADAMT21, IGFBP4, DDR2, HID1, RAPGEF3, AFAP1L1, IL33, PDE2A, GASH1, FEZ1, FERMT2, MAP1B, DLC1, KIAA1462, DPYSL3, PHLDB1, CNN3, CCND1, CDC43IP1, AMOTL2, PTRF, HECW2, MYH10, S100A16, RASIP1, ROBO4, TEAD2, PLK2, MAMA4, BCL6B, KDR, ADGRF5, ARHGEF15, FGD5, SHE, ECSCR, CALCRL, MPDZ, LDB2, APBB2, PTPRB, ARHGAP29, RAI14, TJP1, AKAP12, MYO10, WWTR1, MYO6, SASH1, and SEPT10. Per FIG. 12, the functional categories from top to bottom are Adult_endothelial_progenitor_cell, alternatively_activated_marcrophage (highlighted), band_form_neutrophil, blast_forming_unit_erythroid, CD14-positive_cd16-negative_classical_monocyte, CD3-negative_cd4-positive_cd8-positive_double_positive_thymocyte, CD3-positive_cdr-positive_cd8-positive_double_positive_thymocyte, CD34-negative_cd41-positive_cd43_positive_megakaryocyte_cell, CD38-negative_naïve_b_cell, CD4-positive_alpha_beta_thermocyte, CD4-positive_alpha_beta_t_cell, Cd8-positive_alpha_beta_thermocyte, CD8-positive_alpha_beta_t_cell, central_memory_cd4-positive_alpha_beta_t_cell, central_memory_cd8-positive_alpha_beta_t_cell, class_switched_memory_b_cell, colony_forming_unit_erythroid, common_lymphoid_progenitor, common_myeloid_progenitor, conventional_dendric_cell, cytotoxic_cd56-dim_natural_killer_cell, effector_memory_cd4-positive_alpha_beta_t_cell, effector_memory_cd8-positive_alpha_beta_t_cell, effector_memory_cd8-positive_alpha_beta_t_cell_terminally_differentiated, Endothelial_cell_of_umbilical_vein_(proliferating) (highlighted), Endothelial_cell_of_umbilical_vein_(resting) (highlighted), erythroblast, germinal_center_b_cell, granulocyte_monocyte_progenitor_cell, hematopoietic_multipotent_progenitor_cell, hematopoietic_stem_cell, immature_conventional_dendric_cell, inflammatory_macrophage, late_basophilic_and_polychromatophilic_erythroblast, lymphocyte_of_b_lineage, macrophage, mature_conventional_dendric_cell, mature_eosinophil, mature_neutrophil, megakaryocyte-erythroid_progenitor_cell, memory_b_cell, mesenchymal_stem_cell_of_the_bone_marrow, monocyte, mononuclear_cell_of_bone_marrow_naïve_b_cell, neuroplastic_plasma_cell, neutrophilic_metamyelocyte, neutrophilic nyelocyte, osteoclast, peripheral_blood_mononuclear_cell, plasma_cell, regulatory_t_cell, and segmented_neutrophil_of_bone_marrow, unswitched_memory_b_cell.

TABLE 7
Top 50 Component # 10 Genes from NMF Analysis

| # | Ensembl ID | Gene symbol | Loading fraction |
|---|---|---|---|
| 1 | ENSG00000131016 | AKAP12 | 0.662 |
| 2 | ENSG0000038315 | OIT3 | 0.640 |
| 3 | ENSG00000241644 | INMT | 0.629 |
| 4 | ENSG00000136383 | ALPK3 | 0.617 |
| 5 | ENSG00000183615 | FAM167B | 0.612 |
| 6 | ENSG00000164741 | DLC1 | 0.570 |
| 7 | ENSG00000137033 | IL33 | 0.552 |
| 8 | ENSG00000118407 | FILIP1 | 0.532 |
| 9 | ENSG00000157510 | AFAP1L1 | 0.530 |
| 10 | ENSG00000131711 | MAP1B | 0.529 |
| 11 | ENSG00000145555 | MYO10 | 0.528 |
| 12 | ENSG00000138411 | HECW2 | 0.524 |
| 13 | ENSG0000075618 | FSCN1 | 0.510 |
| 14 | ENSG00000149557 | FEZ1 | 0.509 |
| 15 | ENSG00000154330 | PGM5 | 0.507 |
| 16 | ENSG00000106952 | LHX6 | 0.489 |
| 17 | ENSG00000142748 | FCN3 | 0.488 |
| 18 | ENSG00000110841 | PPFIBP1 | 0.488 |
| 19 | ENSG00000145708 | CRHBP | 0.487 |
| 20 | ENSG00000070778 | PTPN21 | 0.483 |
| 21 | ENSG00000169744 | LDB2 | 0.481 |
| 22 | ENSG00000128052 | KDR | 0.480 |
| 23 | ENSG00000165424 | ZCCHC24 | 0.480 |
| 24 | ENSG00000111961 | SASH1 | 0.470 |
| 25 | ENSG00000177303 | CASKIN2 | 0.468 |
| 26 | ENSG00000071246 | VASH1 | 0.468 |
| 27 | ENSG00000134817 | APLNR | 0.466 |
| 28 | ENSG00000104967 | NOVA2 | 0.465 |
| 29 | ENSG00000186642 | PDE2A | 0.464 |
| 30 | ENSG00000136011 | STAB2 | 0.464 |
| 31 | ENSG00000154783 | FGD5 | 0.463 |
| 32 | ENSG00000163637 | PRICKLE2 | 0.462 |
| 33 | ENSG00000018406 | WWTR1 | 0.460 |
| 34 | ENSG00000118200 | CAMSAP2 | 0.457 |
| 35 | ENSG00000110092 | CCND1 | 0.456 |
| 36 | ENSG00000133392 | MYH11 | 0.456 |
| 37 | ENSG00000198844 | ARHGEF15 | 0.454 |
| 38 | ENSG00000170011 | MYRIP | 0.453 |
| 39 | ENSG00000133026 | MYH10 | 0.453 |
| 40 | ENSG00000069122 | ADGRF5 | 0.452 |
| 41 | ENSG00000074219 | TEAD2 | 0.449 |
| 42 | ENSG00000163697 | APBB2 | 0.449 |
| 43 | ENSG00000188643 | S100A16 | 0.447 |
| 44 | ENSG00000091986 | CCDC80 | 0.446 |
| 45 | ENSG00000249751 | ECSCR | 0.446 |
| 46 | ENSG00000167861 | HID1 | 0.445 |
| 47 | ENSG00000073712 | FERMT2 | 0.445 |
| 48 | ENSG00000074590 | NUAK1 | 0.444 |
| 49 | ENSG00000136830 | FAM129B | 0.443 |
| 50 | ENSG00000172403 | SYNPO2 | 0.443 |

TABLE 8
Component 10 Genes Are Enriched for Blood Vessel
Development Pathways (GO Enrichment Analysis)

| GO Term | Enrichment P value |
|---|---|
| Vasculature development | 1.36E−07 |
| Blood vessel development | 4.49E−07 |
| Angiogenesis | 8.06E−06 |
| Blood vessel morphogenesis | 8.75E−06 |
| Cardiovascular system development | 8.67E−09 |
| Hippo signaling | 1.86E−06 |

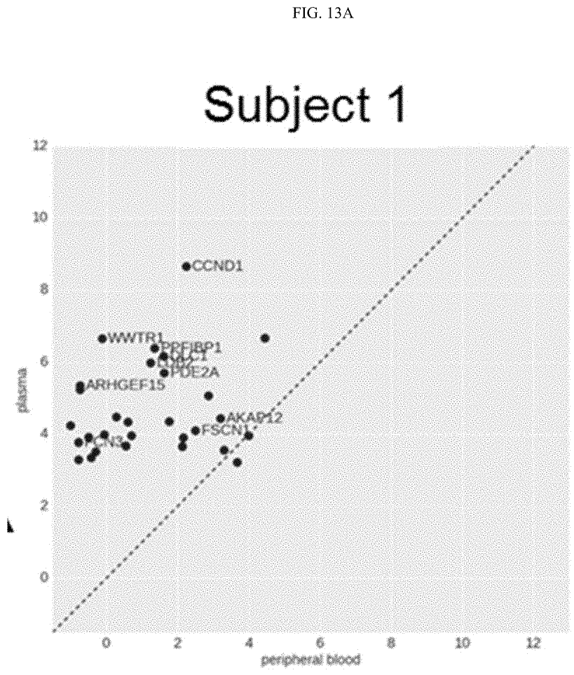

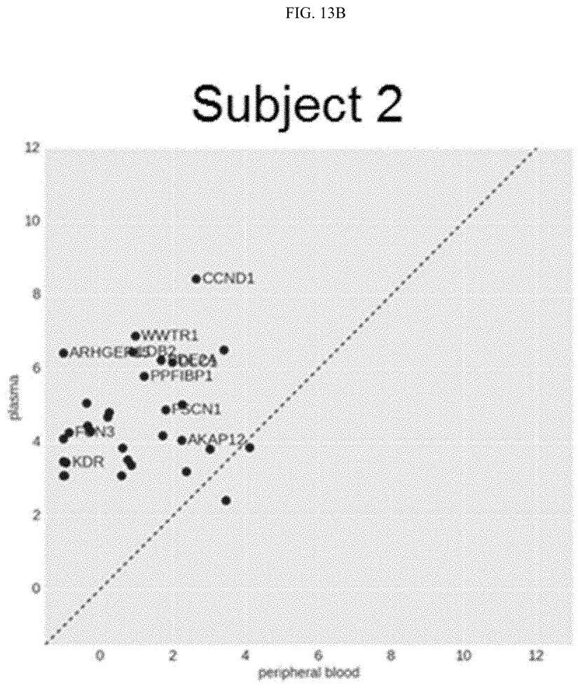

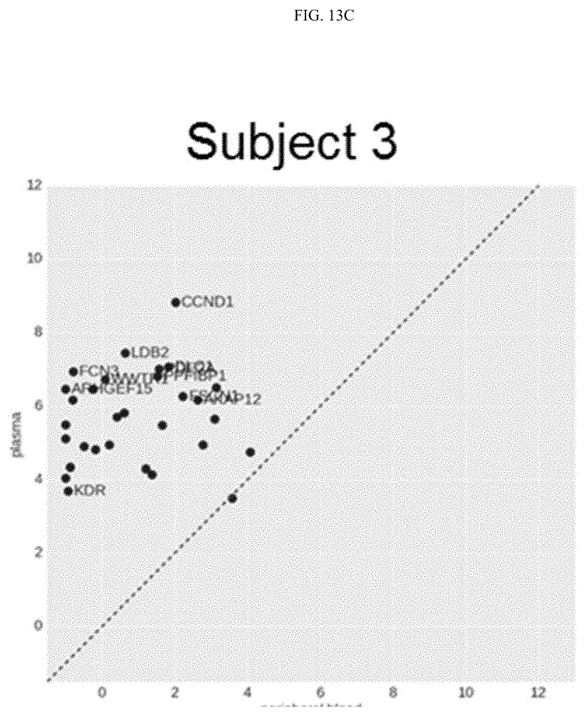

To test the specificity of this blood-vessel development signature to be derived from cf-mRNA (vs. whole-blood cells), expression of genes of NMF-derived component 10 was compared between cf-mRNA of the plasma fraction and peripheral blood, containing all blood-cells (e.g., white blood cells (WBC), neutrophils, platelets, etc.). In 3 non-liver diseased individuals, the genes of NMF component 10 were predominantly present in the plasma fraction of blood (FIGS. 13A-C). These data indicate that specific cf-mRNA gene-expression signatures can provide unique insights into the involvement of blood-vessel development related pathways in liver fibrosis.

Pathway analysis (IPA, Qiagen) was also performed on a set of 500 genes contributing the highest coefficients to the fibrosis classifier. For genes down-regulated in advanced vs. early fibrosis, a strong statistically significant enrichment in genes involved with T-cell responses was observed, such as TCR (T-cell receptor) signaling, the IL-2 and IL-3 cytokine pathways and the downstream JAK pathway (Table 11). Intriguingly, mouse and human studies, have reported IgA producing cells in liver to be responsible for repression of T-cell activation and signaling, and show this repression to cause transitioning of NASH to HCC. Therefore, the observation of repression of T-cell signaling in late stage fibrosis using cf-mRNA, may track with liver disease severity.

TABLE 9
Pathways with Enriched Gene Expression in Early and Advanced Fibrosis

| | P value: Down-regulated | P value: Down-regulated |
|---|---|---|
| Canonical Pathway (IPA; Qiagen) | in Advanced Fibrosis | in Early Fibrosis |
| 1. T Cell Receptor Signaling | 3.83E−07 | 1.20E−01 |
| 2. CD28 Signaling in T Helper Cells | 2.07E−06 | 1.80E−01 |
| 3. IL-2 Signaling | 4.14E−06 | 7.41E−02 |
| 4. IL-3 Signaling | 5.85E−06 | 3.27E−01 |
| 5. JAK/Stat Signaling | 5.85E−06 | 5.15E−02 |
| 6. Acute Myeloid Leukemia Signaling | 4.12E−07 | 3.89E−01 |
| 7. Chronic Myeloid Leukemia Signaling | 9.95E−06 | 3.97E−03 |

Legend: Pathway enrichment of 500 genes with largest Fibrosis classifier coefficients, separated by down-regulated gene-expression (TPM) in advanced (F3, F4) and early fibrosis (F0, F1). P < 10E−5, P < 10E−3 (Fisher's t-test; (IPA; Qiagen).

### Example 2: Application of Cf-mRNA Based NGS Platform to Library Generated from the Cf-RNA of 303 Subjects

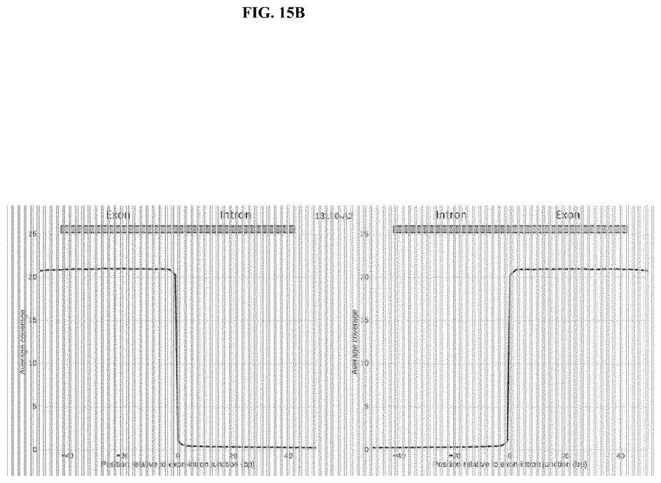

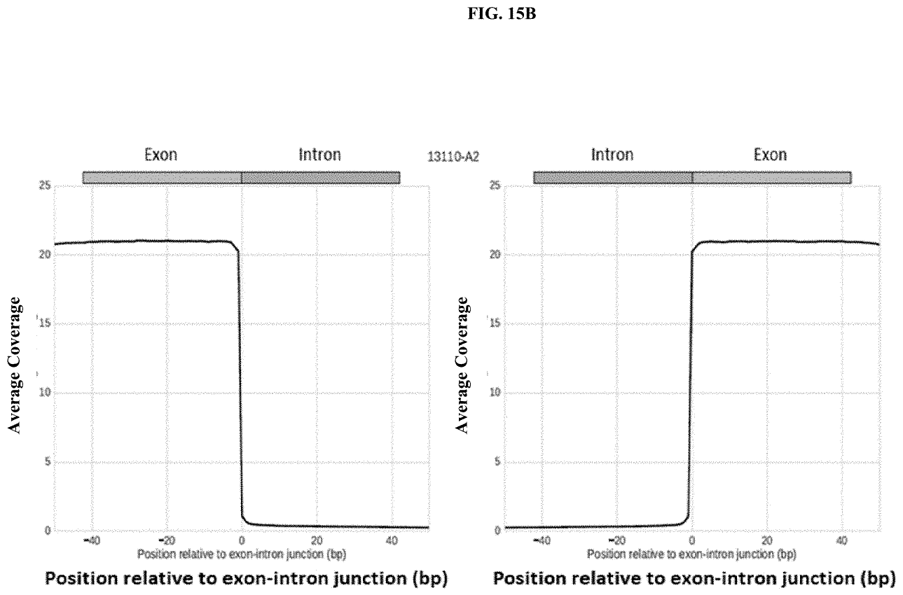

To ensure that the cf-mRNA based NGS platform can be used effectively in liver diseases, technical reproducibility of libraries generated from cf-RNA of 303 subjects was examined. Whole-transcriptome comparison of technical replicates showed high correlation indicative of robust technical reproducibility (log₂ TPM Pearson's correlation >0.9) (FIG. 4A). Moreover, the technical variability of cf-mRNA NGS assay by assessing whole-transcriptome expression across 14 technical replicates of cf-mRNA extracted from a serum sample of a healthy individual (Table 1) was examined. The gene-expression profiles among 14 replicates correlated highly with one another (log 2 TPM, Pearson correlation; mean=0.906, range=0.896-0.914) confirming the reproducibility of the assay. Next, using ERCC (external RNA control consortium) RNA spike-in control, the median sensitivity of the assay was determined to be <100 input RNA molecules (FIG. 4B). Further, the high correlation between observed versus expected number of ERCC molecules demonstrated excellent quantification accuracy (FIG. 4C, FIG. 15A, median Pearson's correlation r >0.95). A median of 8,500 transcripts with greater than 5 TPM (transcripts per million) were identified per sample (FIG. 4D), highlighting the diversity of the information captured by the assay. Gene detection and quantification was not compromised by DNA, as shown by the sharp intron-exon junctions indicating the virtual absence of DNA in the libraries (FIG. 15B). To further validate the approach, the quantification of circulating transcripts obtained by either cf-mRNA sequencing or by multiplex qPCR (Fluidigm BioMark™) (FIGS. 1A and 1B) was compared. RNA isolated from plasma of 61 individuals was split into two aliquots for cf-mRNA NGS and multiplex qPCR profiling, and the expression of 96 genes known to be expressed in healthy and NAFLD liver tissue at different levels was assessed. RNA-seq TPM values inversely correlated with the qPCR cycle threshold (CT) value determined by qPCR (Pearson's correlation, r=−0.86), demonstrating high degree of concordance between cf-mRNA-seq and qPCR (FIGS. 1A and 1B). Together, these data highlight the technical robustness of the cf-mRNA NGS assay.

### Example 3: Identification of NAFLD and Liver Fibrosis Signatures in Circulation

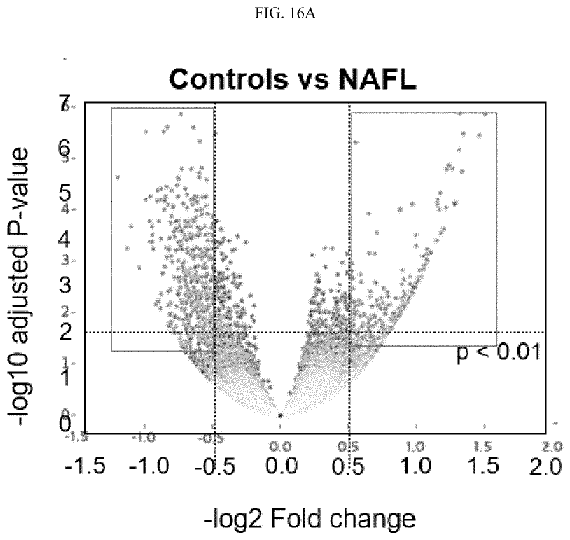

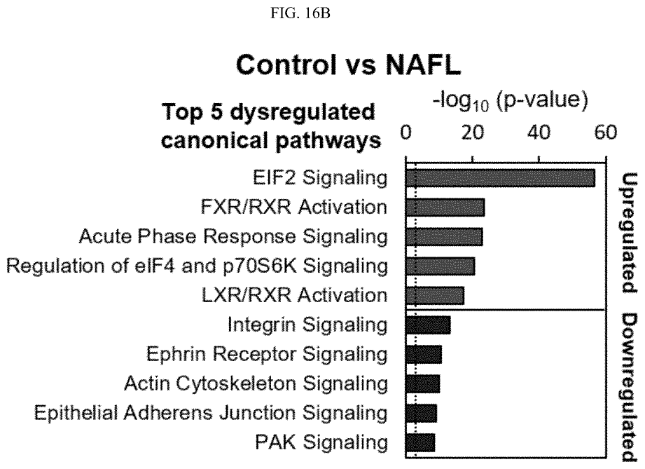

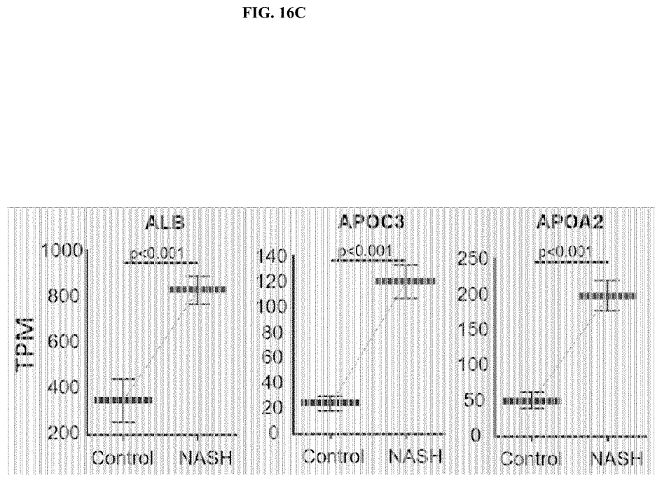

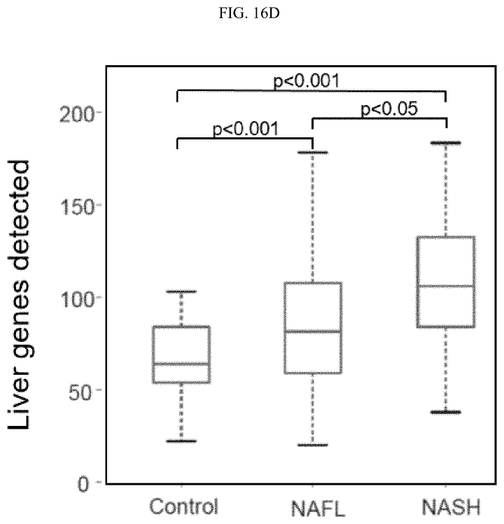

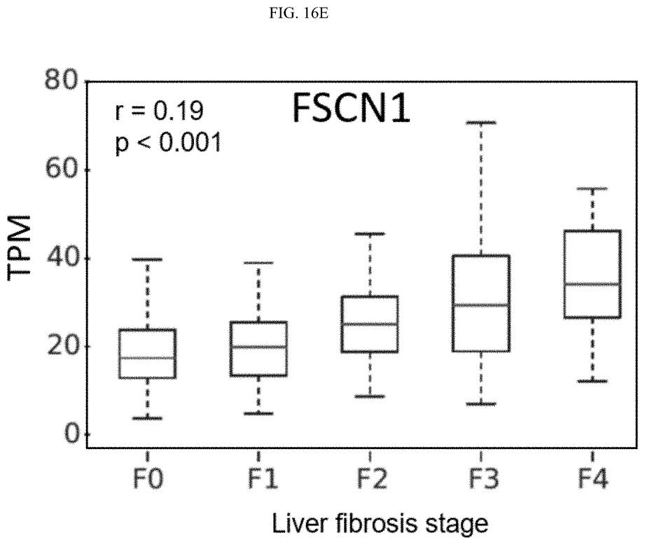

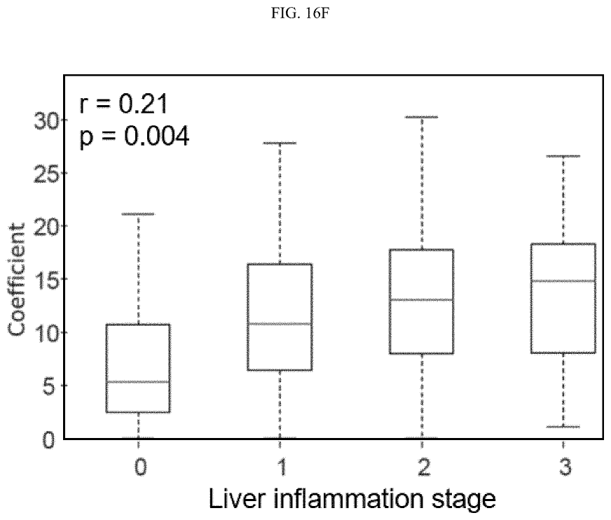

To uncover NAFLD-related transcriptomic changes in serum cf-mRNA, the circulating transcriptomic profiles of control individuals with those of NAFL and NASH patients was compared. Differential expression analysis showed extensive differences between healthy controls and NAFL patients, (1,254 genes dysregulated FDR <0.05) (FIG. 6A) and even more acute differences between controls and NASH patients (2,863 dysregulated genes FDR <0.05, FIG. 6A). Functional analyses of the dysregulated genes with IPA revealed that the genes upregulated in NAFLD patients are enriched mainly in liver associated pathways, such as the pleiotropic LXR/RXR and FXR/RXR signaling pathways, involved in cholesterol, triglyceride and glucose metabolism, and acute phase response reflective of liver injury and/or inflammation (FIG. 6B and FIG. 16B). Almost all liver-specific transcripts (e.g., Albumin, APOA2, APOC3) detected in cf-mRNA were up-regulated in the serum of NASH patients compared to control samples (FIG. 6C and FIG. 16C). Moreover, a significant increase in the number of liver-specific genes detected in the serum of NASH and NAFL patients was observed compared to those of control individuals (FIG. 16D). The observed dysregulation of liver specific genes and pathways indicate that cf-mRNA reflects NAFLD associated pathological changes in the liver.

To better understand the information captured by the circulating transcriptome, the correlation patterns of the cf-mRNA transcripts were investigated. Unsupervised decomposition of cf-mRNA transcriptomes by non-negative Matrix Factorization (NMF) using all the samples and subsequently identified 12 distinct gene-clusters, many of which are enriched in certain cell types or biological processes (FIG. 16B) was performed. Functional analysis of the five most prominent clusters is shown in FIG. 6D. First, consistent with the previous analyses, it was found that genes in Cluster 7 (n=664 genes) were enriched in liver related pathways (FIG. 2D). Indeed, 63% of the top 100 genes in Cluster 7 are liver specific (p-value <1e-11, hypergeometric test) according to GTEx database (21), suggesting that these genes originated from hepatocytes in the liver (FIG. 6D). Second, the top enriched IPA pathway for genes in Cluster 5 (n=527 genes) was “hepatic stellate cell activation” (p<0.0001), the central biological event of hepatic fibrosis (REF). Further supporting the association of these genes with liver fibrosis, the coefficients of this cluster correlated with the fibrosis score of the NAFLD patients assessed by liver biopsy (r=0.23 and p<0.001). Third, genes in Cluster 11 (n=510) were enriched in inflammatory processes, with the most significant canonical pathway being “interferon signaling” (p<0.001) (FIG. 6D). Without being bound by any one particular theory, these genes may reflect liver inflammation, as their levels correlated with the histological inflammation score determined by liver biopsy (r=0.21 and p=0.004, FIG. 16F). In summary, these results indicate that cf-mRNA transcriptome profiling non-invasively uncovers the tissues, cell types, biological processes and signaling pathways dysregulated in NAFLD.

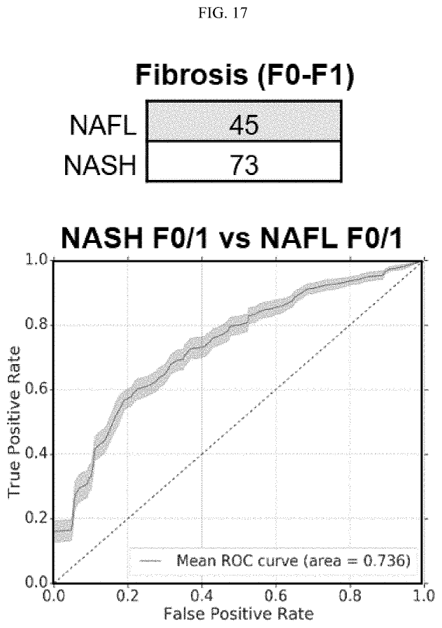

Since progressive scarification of the liver is a main factor associated in NAFLD patients with chronic liver complications, transcriptomic signatures of advanced fibrosis were identified by comparing the circulating transcriptomes of patients with mild (F0-F1) and advanced (F3-F4) liver fibrosis determined by biopsy. 253 dysregulated genes were identified (FDR <0.05, FIG. 6E) and demonstrated that genes upregulated in patients with severe liver scarring were enriched in signaling pathways involved in liver fibrosis onset and progression such as “hepatic fibrosis/hepatic stellate cell activation” and “PI3K/AKT signaling pathway” (REF). Furthermore, since liver fibrosis is a progressive process featured by different levels of severity, de novo identification of genes whose cf-mRNA levels correlate with fibrosis stage was performed. 613 such transcripts were found in circulation (FDR<0.05, FIG. 17). The gene showing the best linear correlation with fibrosis stage, FSCN1 (FIG. 16E), encodes a member of actin-bundling proteins and has recently been identified as a marker for hepatic stellate cells. Further, the expression of FSCN1 positively regulates the expression of collagens and matrix metalloproteinase (REF), which is consistent with the observation of steadily increasing levels of FSCN1 in patients with progressively more severe hepatic fibrosis.

### Example 4: Development of cf-mRNA NAFLD Diagnostic Classifiers

cf-mRNA profiling can be used to build diagnostic classifiers to discriminate NAFL and NASH patients from heathy individuals. The training cohort was randomly divided into a “training-set” (60% of the samples) where a cf-mRNA-based classifier was trained using various machine learning models, and a “testing set” (remaining 40% of the samples). The receiver operating characteristic (ROC) curves and associated area under the curve (AUC) values were calculated to assess the performance of each classification (FIG. 8A). This process was repeated 15 times to obtain a sample of AUCs and unbiased evaluation of model generalizability. Top 50 genes for discriminating normal controls vs NASL are listed in Table 3. Top 50 genes for discriminating normal controls vs NASH are listed in Table 4. The cf-mRNA diagnostic classifiers robustly discriminated NAFL and NASH from healthy individuals (average AUC=0.92 and 0.93 respectively, FIGS. 8A and 8B).

One of the fundamental constraints of the current fibrosis tests is their limited ability to differentiate NASH from simple steatosis. The information captured in the cf-mRNA transcriptome was used to discriminate among these patients. A cf-mRNA classifier was built that distinguishes NAFL patients from NASH patients with an average AUC of 0.77 ((95% CI: 0.74.0.78), FIG. 8D). Furthermore, NASH patients with mild liver fibrosis represent a high risk population that would benefit from close monitoring, but are generally undetectable by current non-invasive methods. A classifier was developed to discriminate NASH from simple steatosis among patients with low-grade fibrosis (F0-1) (n=73 patients were diagnosed with NASH among the 118 patients with low fibrosis), with an average AUC of 0.74 (FIG. 17). Top 50 genes for discriminating NAFL vs NASH are listed in Table 5.

### Example 5: Stratification of NAFLD Fibrosis Stages

A “training” (n=188) and a “validation” cohort (n=60) of patients with fibrosis stage determined by liver biopsy (FIGS. 14B-14C) were collected. A classifier for the discrimination of advanced (F3-F4) from mild (F0-F1) fibrosis in biopsy validated patients of the training cohort was developed. A classifier for the discrimination of mild (F0-F1) was also developed (FIG. 17). Cross validation within the retrospective training cohort showed an average AUC of 0.81 (FIG. 14A). Top 50 genes for discriminating early vs late fibrosis are listed in Table 10.

TABLE 10
Early v. Late Fibrosis

| # | Gene name | Ensembl Gene ID |
|---|---|---|
| 1 | TNFAIP8L1 | ENSG00000185361 |
| 2 | E2F1 | ENSG00000101412 |
| 3 | CDC42EP1 | ENSG00000128283 |
| 4 | INMT | ENSG00000241644 |
| 5 | NT5DC2 | ENSG00000168268 |
| 6 | FSCN1 | ENSG00000075618 |
| 7 | EVA1B | ENSG00000142694 |
| 8 | MLKL | ENSG00000168404 |
| 9 | ZNF462 | ENSG00000148143 |
| 10 | DRAM1 | ENSG00000136048 |
| 11 | TRIB3 | ENSG00000101255 |
| 12 | LZTR1 | ENSG00000099949 |
| 13 | EPB41L4A | ENSG00000129595 |
| 14 | RNF25 | ENSG00000163481 |
| 15 | FAM127B | ENSG00000203950 |
| 16 | ZNF438 | ENSG00000183621 |
| 17 | ACAD9 | ENSG00000177646 |
| 18 | RASAL2 | ENSG00000075391 |
| 19 | ANKRD55 | ENSG00000164512 |
| 20 | WBP5 | ENSG00000185222 |
| 21 | KCTD13 | ENSG00000174943 |
| 22 | CD33 | ENSG00000105383 |
| 23 | FMNL2 | ENSG00000157827 |
| 24 | RP11-400F19.6 | ENSG00000266962 |
| 25 | GRAMD4 | ENSG00000075240 |
| 26 | PLCB3 | ENSG00000149782 |
| 27 | GALNT10 | ENSG00000164574 |
| 28 | KALRN | ENSG00000160145 |
| 29 | CTTNBP2NL | ENSG00000143079 |
| 30 | ING5 | ENSG00000168395 |
| 31 | MYO10 | ENSG00000145555 |
| 32 | NOVA2 | ENSG00000104967 |
| 33 | AGPAT5 | ENSG00000155189 |
| 34 | IFFO1 | ENSG00000010295 |
| 35 | ZHX3 | ENSG00000174306 |
| 36 | FRMD3 | ENSG00000172159 |
| 37 | HYAL2 | ENSG00000068001 |
| 38 | C8orf4 | ENSG00000176907 |
| 39 | ANKRD46 | ENSG00000186106 |
| 40 | GNA12 | ENSG00000146535 |
| 41 | CREB3L2 | ENSG00000182158 |
| 42 | ZNF561 | ENSG00000171469 |
| 43 | TOR1AIP1 | ENSG00000143337 |
| 44 | FEZ1 | ENSG00000149557 |
| 45 | PSMB5 | ENSG00000100804 |
| 46 | SEH1L | ENSG00000085415 |
| 47 | NCKAP5L | ENSG00000167566 |
| 48 | MLLT4 | ENSG00000130396 |
| 49 | RBPMS | ENSG00000157110 |
| 50 | FAM114A1 | ENSG00000197712 |

Subsequently, this classifier in prospectively collected serum samples was validated. By comparing the predictions from the classifier with the true fibrotic stages obtained from liver biopsy, the robustness of the fibrosis stratification classifier (AUC=0.83 (95% CI: 0.71-0.95), FIG. 14B) was confirmed. To evaluate the clinical utility of the cf-mRNA fibrosis stratification classifier, several clinical parameters were examined. In the prospective validation cohort, at a set specificity level of ˜80%, the sensitivity of the classifier was ˜80% (FIG. 18). As the range of referral rate varies considerably between medical centers, the positive- and negative-predictive values (PPV and NPV) of the classifier over a range of patient referral rates (FIG. 18) was also evaluated. The PPV of >80% indicates the potential of cf-mRNA to stratify NAFLD patients by their liver fibrosis stage (FIG. 14C).

Further, the patient eligibility criteria for future NASH therapies may include both the NASH status and the liver fibrosis stage. For example, a clinical trial that showed promising results in the Phase 3 trials involve patients with NASH and have fibrosis stages 2 or higher could pursue patient selection by a classifier to identify specifically patients with >F2 due to NASH with an AUC of 0.74. In a pre-enriched population where at least 40% of the patients have NASH and F2 or higher, the PPV of the test would be 88% (cutoff of 0.4).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of assessing a disease state of a liver, the method comprising:
(a) obtaining or having obtained a sample from a subject, wherein the sample comprises gene expression products;
(b) measuring gene expression products of a panel of genes comprising at least one gene selected from Table 3, Table 4, Table 5, Table 6, Table 7 and/or Table 10; and
(c) determining a disease state of the liver.

1.-84. (canceled)
